(19) United States
(12) Patent Application Publication
(10) Pub. No.: US 2022/0289657 A1
(43) Pub. Date: Sep. 15, 2022

(54) COMPOSITION FOR PREVENTING OR TREATING BACTERIAL INFECTIOUS DISEASE COMPRISING 4-GINGEROL DERIVATIVE COMPOUND AS ACTIVE INGREDIENT

(71) Applicants: KOREA UNIVERSITY RESEARCH AND BUSINESS FOUNDATION, SEJONG CAMPUS, Sejong (KR); KOREA UNIVERSITY RESEARCH AND BUSINESS FOUNDATION, Seoul (KR)

(72) Inventors: Youngjoo BYUN, Sejong (KR); Hee Deung PARK, Seongnam-si (KR); Sangjin NAM, Sejong (KR); Sang-Hyun SON, Sejong (KR); So Young HAM, Seongnam-si (KR); Han Shin KIM, Siheung-si (KR)

(73) Assignees: KOREA UNIVERSITY RESEARCH AND BUSINESS FOUNDATION, SEJONG CAMPUS, Sejong (KR); KOREA UNIVERSITY RESEARCH AND BUSINESS FOUNDATION, Seoul (KR)

(21) Appl. No.: 17/637,565

(22) PCT Filed: Aug. 21, 2020
(86) PCT No.: PCT/KR2020/011180

§ 371 Date: Apr. 27, 2022

(30) Foreign Application Priority Data
Aug. 23, 2019 (KR) 10-2019-0103522

(51) Int. Cl. C07C 49/255; C07C 49/248; C07C 49/235; A61K 31/12; A61P 31/04

(52) U.S. Cl. CPC C07C 49/255; C07C 49/248; C07C 49/235; A61K 31/12; A61P 31/04

(57) ABSTRACT

The present invention relates to a 4-gingerol derivative compound, or a racemate, isomer, or pharmaceutically acceptable salt thereof, the compound being capable of inhibiting biofilm formation and production of virulence factors. The 4-gingerol derivative compound according to the present invention has binding affinity to RhlR and corresponding RhlR antagonism activity that are significantly improved, and therefore can effectively inhibit biofilm formation and production of virulence factors. Furthermore, various bacterial infectious diseases caused by biofilms can be fundamentally prevented or treated by using a pharmaceutical composition comprising the 4-gingerol derivative compound, or a racemate, isomer, or pharmaceutically acceptable salt thereof as an active ingredient.

### TECHNICAL FIELD

The present invention relates to a 4-gingerol derivative compound that can inhibit the formation of a biofilm and the production of a virulence factor, or a racemate, isomer, or pharmaceutically acceptable salt thereof.

### BACKGROUND ART

Pseudomonas aeruginosa is a human pathogen that induces a chronic disease in immunodeficiency patients, and causes infections in burn wounds, cystic fibrosis, acute leukemia, organ transplantation, and intravenous drug addiction. According to the World Health Organization (WHO), since Pseudomonas aeruginosa, as one of major pathogens requiring close attention, generally forms a thick biofilm, it has resistance to ordinary antibiotics, and specifically resides in large quantities on various surfaces in the form of biofilms covered with self-produced exopolysaccharides. Biofilms are widely found medically, dentally, agriculturally, industrially and environmentally, and particularly, medically, biofilms are associated with approximately 80% of human bacterial infections, and have increased antibiotic resistance.

Quorum sensing (QS) is a bacterial cell-to-cell communication process via a chemical signaling molecule to allow bacteria to share information with each other in response to environmental changes. When a bacterial density reaches a certain threshold, chemical signaling molecules bind to their cognate receptor proteins to modulate collective behaviors, and change gene expression. Pseudomonas aeruginosa has three major QS systems, which are tightly connected to each other. Gram-negative bacteria including Pseudomonas aeruginosa use N-acyl L-homoserine lactones (AHLs) as signaling molecules in the QS systems. Typically, AHLs are produced by LuxI-type synthases (e.g., LasI and RhiI), and recognized by cytoplasmic LuxR-type receptors (e.g., LasR and RhlR). LasI and RhlI are signaling molecules, which produce N-(3-oxo-dodecanoyl)-L-homoserine lactone (OdDHL, Compound 1a) and N-butyryl-L-homoserine lactone (BHL, Compound 1b), respectively (FIG. 1). The pqs circuit is the third QS system, and regulated by a Pseudomonas quinolone signal (PQS) recognized by pqsR. The three systems are hierarchically regulated. The las system activates the rhl and pqs systems, and the rhl system inhibits the pqs system. The reciprocal signal-network regulation of Pseudomonas aeruginosa forms biofilms, produces a virulence factor, and induces the regulation of a host immune response.

Most QS system inhibitors of Pseudomonas aeruginosa focus on targeting LasR because the las system is signally located at the uppermost part of the QS system of Pseudomonas aeruginosa. On the other hand, although rhl plays an important role in the QS system, little has been reported about an RhlR modulator. Recently, as a modulator for the interaction between BHL and RhlR, some agonists or antagonists have been reported. As an example, a BHL analog having RhlR agonist activity reduces the production of a Pseudomonas aeruginosa virulence factor such as pyocyanin, and a mixture of LasR/RhlR antagonists, which increases pyocyanin production, were disclosed. In addition, from an experiment using a BHL-independent rhlR variant, it was revealed that RhlR induces biofilm formation and virulence factor production in Pseudomonas aeruginosa.

Meanwhile, in previous research, the present inventors revealed that (S)-6-gingerol (Compound 1c, FIG. 1) extracted from ginger can be used as an LasR antagonist of Pseudomonas aeruginosa since it forms a biofilm, produces a virulence factor, and expresses a QS-related gene.

Under the above technical background, during research on the structure-activity relationship (SAR) for a gingerol analog, the present inventors confirmed that gingerol with a shorter alkyl chain than (S)-6-gingerol is structurally similar to BHL and thus more easily binds to RhlR, and as a pure RhlR antagonist, the present invention including a potent RhlR antagonist inhibiting biofilm formation and virulence factor production by widely altering a chemical structure in consideration of the design, synthesis and biochemical characteristics of a gingerol analog was completed.

### DISCLOSURE

### Technical Problem

The present invention is directed to providing a 4-gingerol derivative compound, or a racemate, isomer or pharmaceutically acceptable salt thereof, which can inhibit the formation of a biofilm and the production of a virulence factor.

The present invention is also directed to providing a composition for inhibiting biofilm formation, which includes the gingerol derivative compound, or a racemate, isomer or pharmaceutically acceptable salt thereof as an active ingredient.

The present invention is also directed to providing a pharmaceutical composition for preventing, treating or diagnosing a bacterial infection, which includes the gingerol derivative compound, or a racemate, isomer or pharmaceutically acceptable salt thereof as an active ingredient.

However, technical problems to be solved in the present invention are not limited to the above-described problems, and other problems which are not described herein will be fully understood by those of ordinary skill in the art from the following descriptions.

### Technical Solution

To solve the above-described problems, the present invention provides a 4-gingerol derivative compound represented by Formula 1 below, or a racemate, isomer, enantiomer, or pharmaceutically acceptable salt thereof.

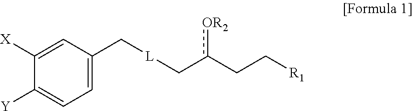

According to one embodiment of the present invention, in Formula 1, X and Y may be the same or different from each other, and may each be independently any one selected from hydrogen, a halogen group, an alkyl group, a hydroxyl group, O—R′, and NR′R′, R′ my be a C1-C2 alkyl group, R₁ may be a C1-C3 alkyl group, the “_” represents a single bond or a double bond or a triple bond, when the “_” is a double bond, OR₂ is O, and when the “_” is a single bond, OR₂ is OH, and L may be any one selected from a single bond, a double bond and a triple bond.

According to another embodiment of the present invention, an isomer of the gingerol derivative compound may be any one selected from the group consisting of an (R)-configuration and an (S)-configuration.

According to still another embodiment of the present invention, an isomer of the gingerol derivative compound may be any one selected from the group consisting of an (E) isomer or a (Z) isomer.

In addition, the present invention provides a 4-gingerol derivative compound represented by Formula 2 below, or a racemate, isomer or pharmaceutically acceptable salt thereof.

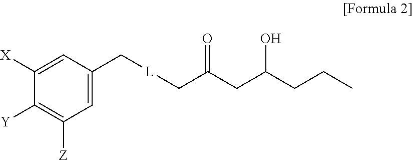

According to one embodiment of the present invention, in Formula 2, X, Y and Z may be the same or different from each other, and may each be independently any one selected from hydrogen, a halogen group, an alkyl group, a hydroxyl group, O—R′, and NR′R′, R′ may be a C1-C2 alkyl group, and L may be a single bond or a double bond.

According to another embodiment of the present invention, the gingerol derivative represented by Formula 2 may be any one selected from combinations of X. Y and Z as below.

(1) X═OMe, Y═OH, Z═H

(2) X═H, Y═H, Z═H

(3) X═OMe, Y═H, Z═H

(4) X═H, Y═OH, Z═H

(5) X═OH, Y═H, Z═H

(6) X═H, Y═OMe, Z═H

(7) X═H, Y═F, Z═H

(8) X═H, Y═Cl, Z═H

(9) X═H, Y=Me, Z═H

(10) X═H, Y═NMe₂, Z═H

(11) X═OH, Y═OH, Z═H

(12) X═OMe, Y═OMe, Z═H

(13) X═F, Y═OH, Z═H

(14) X═OEt, Y═OH, Z═H

(15) X═F, Y═F, Z═H

(16) X=Me, Y=Me, Z═H

(17) X═OMe, Y═F, Z═H

(18) X═F, Y═H, Z═OMe

In addition, the present invention provides a composition for inhibiting biofilm formation, which includes the 4-gingerol derivative compound represented by Formula 1 or 2, or a racemate, isomer or pharmaceutically acceptable salt thereof as an active ingredient.

According to one embodiment of the present invention, the biofilm may be formed by one or more species of bacteria selected from the group consisting of Pseudomonas aeruginosa, Salmonella spp, Shigella spp, Vibrio parahaemolyticus, Vibrio choreae, Escherichia coli O-157, Campylobacter jejuni, Clostridium dificile, Clostridium perfringens, Yersinia enterocolitica, Helicobacter pylori, Entemoeba histolytica, Bacillusu cereus, Clostridium botulinum, Haemophilus influenzae, Streptococcus pneumoniae, Chlamidia pneumoniae, Legionella pneumoniae, Branhamella catarrhalis, Mycobacterium tuberculosis, Mycoplasma pneumoniae, Group A Streptococcus (Streptococcus pyogenes), Corynebacterium diphtheriae, Bordetella pertussis, Chramidia psittaci, methicillin resistant Staphylococcus aureus (MRSA), Escherichia coli, Klebsiella pneumoniae, Enterobacter spp, Proteus spp, Acinetobacter spp, Enterococcus faecalis, Staphylococcus saprophyticus, and Group B Streptococcus (Streptococcus agalactiae).

According to another embodiment of the present invention, the 4-gingerol derivative compound is structurally similar to BHL, and has RhlR-binding affinity. Due to relatively high RhlR antagonistic activity, the 4-gingerol derivative compound can inhibit the formation of a biofilm and the production of a virulence factor.

In addition, the present invention provides a pharmaceutical composition for preventing or treating a bacterial infection, which includes the 4-gingerol derivative compound represented by Formula 1 or 2, or a racemate, isomer or pharmaceutically acceptable salt thereof as an active ingredient.

According to one embodiment of the present invention, the bacterial infection may be one or more types of infectious diseases selected from the group consisting of cystic fibrosis, pneumonia, tooth decay, periodontitis, otitis media, musculoskeletal infection, necrotizing fasciitis, biliary tract infection, osteomyelitis, bacterial prostatitis, native valve endocarditis, melioidosis, nosocomial infection, ICU pneumonia, urinary catheter cystitis, continuous ambulatory peritoneal dialysis (CAPD) peritonitis, and biliary stent blockage.

According to another embodiment of the present invention, the 4-gingerol derivative compound inhibits the formation of a biofilm and the production of a virulence factor by downregulating a rhamnolipid synthesis gene rhlA.

In addition, the present invention provides a method of preventing or treating a bacterial infection, which includes administering the 4-gingerol derivative compound represented by Formula 1 or 2, or a racemate, isomer or pharmaceutically acceptable salt thereof into a subject.

### Advantageous Effects

The present invention relates to a 4-gingerol derivative compound which can inhibit the formation of a biofilm and the production of a virulence factor, or a racemate, isomer or pharmaceutically acceptable salt thereof. The 4-gingerol derivative compound according to the present invention is significantly improved in binding affinity to RhlR and RhlR antagonistic activity thereby, so it can inhibit the formation of a biofilm and the production of a virulence factor. In addition, a pharmaceutical composition including the gingerol derivative compound, or a racemate, isomer or pharmaceutically acceptable salt thereof as an active ingredient can be used to fundamentally prevent or treat various types of bacterial infections caused by a biofilm.

### DESCRIPTION OF DRAWINGS

FIG. 1 shows the structures of an endogenous molecule and a synthetic molecule, which interact with a Quorom sensing (QS) receptor of Pseudomonas aeruginosa.

FIG. 2 shows the chiral resolution of 4-gingerol derivative compounds 23a-23c.

FIG. 3 shows the chiral HPLC results, showing the chiral resolution of 3,4-difluorophenyl derivatives, such as (A) compounds 33a-33c, (B) compounds 34a-34c, and (C) compounds 35a-35c.

FIG. 4 shows docked poses of Compound 30 (yellow) and Compound 1d (pink) along with an RhlR homology model. Red and black dashed lines represent the p-p stacking interaction and hydrogen bonds, respectively.

FIG. 5 shows the docked poses of alkynyl ketones (Compounds 30-32) along with an RhlR homology model. (A) represents a docked pose of Compound 30, (B) represents a docked pose of Compound 31, and (C) represents a docked pose of Compound 32.

FIG. 6 shows the in vitro biological activity of Compounds 1c, 1d, 12a and 30. DMSO was used as a negative control, and Compounds 1c, 1d and 12a were used as positive controls. (A) is a relative RhlR activity dose-response curve, (B) shows the agonistic activity of relative QS receptors (LasR and PqsR) at 0.1 and 10 μM of the compounds, respectively, (C) shows the relative QS receptor (LasR and PqsR) antagonistic activity at 0.1 and 10 μM of the compounds, respectively, and (D) shows the static biofilm formation of Pseudomonas aeruginosa at 0.1 and 10 μM of the compounds ((**) P<0.005 compared to control, and (*) P<0.05 compared to control).

FIG. 7 shows the Pseudomonas aeruginosa growth when 10 or 100 μM of Compound 1c, 1d, 12a or 30 is treated under a static condition. DMSO was used as a negative control (C), and Compounds 1c, 1d and 12a were used as positive controls ((**) P<0.005 compared to control, and (*) P<0.05 compared to control).

FIG. 8 shows the biofilm formation using 10 μM of Compounds 1c, 1d and 30 under a flow condition in Pseudomonas aeruginosa. DMSO was used as a negative control, and Compounds 1c, 1d and 12a were used as positive controls. (A) shows confocal laser scanning microscopic (CLSM) images of the biofilms, representing volumes and thicknesses, (B) shows carbohydrate amounts in EPS, and (C) shows protein amounts in EPS ((**) P<0.005 compared to control, and (*) P<0.05 compared to control).

FIG. 9 shows the expression of QS-induced genes and the production of virulence factors by Pseudomonas aeruginosa when Compounds 1c, 1d, 12a and 30 are treated. DMSO was used as a negative control, and Compounds 1c, 1d and 12a were used as positive controls. (A) shows rhamnolipid production by wild-type Pseudomonas aeruginosa according to the treatment with 10 or 100 μM of the compounds, (B) shows the relative rhlA expression of biofilm cells according to the treatment with 10 μM of the compounds, (C) shows pyocyanin production by wild-type Pseudomonas aeruginosa and rhlR variants according to the treatment with 10 μM of the compounds, and (D) shows the relative QS-related gene expression of wild-type Pseudomonas aeruginosa and rhlR variants ((**) P<0.005 compared to control, and (*) P<0.05 compared to control).

FIG. 10 shows the mortality of Pseudomonas aeruginosa-injected T. molitor according to the treatment with 10 μM of Compounds 1c, 1d, 12a or 30. DMSO was used as a negative control, and Compounds 1c, 1d and 12a were used as positive controls.

FIG. 11 shows the mortality of Pseudomonas aeruginosa-injected T. molitor larvae when various concentrations (0.1-10 μM) of a rhamnolipid are treated. DMSO was used as a negative control.

FIG. 12 shows a working model for controlling the QS system of Pseudomonas aeruginosa according to the treatment with Compound 30.

### MODES OF THE INVENTION

As a result of earnest research on a QS system modulator for inhibiting biofilm formation and virulence factor production, the present inventors confirmed the rhl-RhlR antagonistic activity of 4-gingerol analogs, and the present invention was completed.

More specifically, the present inventors confirmed that (1) since gingerol having a shorter alkyl chain than (S)-6-gingerol is structurally similar to BHL, it more easily binds to RhlR, (2) as a substituent at position 3 of the phenyl ring is smaller, and preferably, the substituent is F, stronger RhlR antagonistic activity is shown, (3) as a substituent at position 4 of the phenyl ring, which is a site having a hydrogen bond with RhlI, is a polar substituent, and preferably, the substituent is F or OH, it has a stronger binding strength to RhlR, (4) when compounds have a triple bond, a double bond and a single bond between a phenyl ring and a carbonyl group, they are more potent RhlR antagonists, (5) an (E)-alkenyl compound has stronger RhlR antagonistic activity than a (Z)-alkenyl compound, and (6) a carbonyl group or hydroxyl group located at the γ-position from a phenyl group is important for binding to RhlR.

From the above results, the present inventors found that, as the 4-gingerol derivative compound represented by Formula 1 or 2 has a strong binding affinity to RhlR, it has RhlR antagonistic activity, and is able to inhibit biofilm formation and virulence factor production. Therefore, the present invention may provide a pharmaceutical composition for preventing, treating or diagnosing a bacterial infection, which includes the 4-gingerol derivative compound represented by Formula 1 or 2, or a racemate, isomer or pharmaceutically acceptable salt thereof as an active ingredient.

The term “prevention” used herein refers to all actions of inhibiting or delaying the occurrence, spread or recurrence of a bacterial infection by the administration of the composition of the present invention, and the “treatment” refers to all actions involved in alleviating or beneficially changing symptoms of the disease by the administration of the composition of the present invention.

The term “pharmaceutical composition” used herein refers to one that is prepared to prevent or treat a disease, and may be used by being formulated in various forms according to a conventional method. For example, the pharmaceutical composition may be formulated as an oral formulation such as a powder, a granule, a tablet, a capsule, a suspension, an emulsion or a syrup, and in the form of a preparation for external use, a suppository or a sterile injectable solution.

In the present invention, the 4-gingerol derivative compound represented by Formula 1 or 2 or a racemate or isomer thereof may be used in the form of a pharmaceutically acceptable salt, which means the formulation of a compound that does not cause serious irritation to an organism administered the compound and does not impair the biological activity and physical properties of the compound. The pharmaceutically acceptable salt may be obtained by reaction of the compound of the present invention with an inorganic acid such as hydrochloric acid, hydrobromic acid, sulfuric acid, nitric acid or phosphoric acid, a sulfonic acid such as methanesulfonic acid, ethanesulfonic acid or p-toluenesulfonic acid, or an organic carbonic acid such as tartaric acid, formic acid, citric acid, acetic acid, trichloroacetic acid, trifluoroacetic acid, capric acid, isobutanoic acid, malonic acid, succinic acid, phthalic acid, gluconic acid, benzoic acid, lactic acid, fumaric acid, maleic acid or salicylic acid. In addition, the pharmaceutically acceptable salt may be obtained by forming a salt, for example, an alkali metal salt such as an ammonium salt, or a sodium or potassium salt, an alkaline earth metal salt such as a calcium or magnesium salt, a salt of an organic base such as dicyclohexylamine, N-methyl-D-glucamine, or tris(hydroxymethyl)methyl amine, and an amino acid salt such as arginine or lysine by the reaction of the compound of the present invention with a base.

In addition, the 4-gingerol derivative compound represented by Formula 1 or 2 or a racemate or isomer thereof may include not only a pharmaceutically acceptable salt, but also all salts, hydrides and solvates which may be prepared by a conventional method.

In the present invention, a corresponding component is “included as an active ingredient” means that the component is included as much as needed or at a sufficient amount to implement a desired biological effect. In practical application, the amount included as an active ingredient is an amount for treating a target disease, which may be determined by taking into account that it does not cause other toxicity, and may be changed according to various factors, for example, a disease or condition to be treated, the form of a composition to be administered, the size of a subject, or the severity of a disease or condition. The effective amount of an individual composition may be empirically determined by those of ordinary skill in the art to which the present invention belongs without accompanying undue experimentation.

In the present invention, the bacterial infection may be an infectious disease that can be inhibited by an antibacterial effect, and may include infectious diseases treated with conventional antiseptics and antibiotics and a case in which an infectious disease can be induced in the diagnosis of a disease. Examples of the bacterial infections may include cystic fibrosis, pneumonia, tooth decay, periodontitis, otitis media, musculoskeletal infection, necrotizing fasciitis, biliary tract infection, osteomyelitis, bacterial prostatitis, native valve endocarditis, melioidosis, nosocomial infection, ICU pneumonia, urinary catheter cystitis, continuous ambulatory peritoneal dialysis (CAPD) peritonitis, and biliary stent blockage.

In the present invention, a position that can form a biofilm may be a suture site, an excision site, an arteriovenous site, a scleral buckle site, contact lenses, an IUD, a tracheal tube, a Hickman catheter, a central venous catheter, an artificial heart valve, a valve graft, an orthopedic device or a penile implant, and other examples are disclosed in the article written by Costerton J et al. and Costerton J and Steward (2001 Battling Biofilms, Scientific American pp 75-81), which may be incorporated herein by reference. Other positions at which a biofilm can be formed may include dental caries that may develop gum disease and a cavity, contact lenses in which an infection may occur, ears in which a chronic infection may occur, and the lungs that may develop pneumonia.

In the present invention, the infection may be cystic fibrosis and may be caused by a skin infection, a burn infection, and/or a wound infection. The composition of the present invention may be particularly suitable for treating an infection in immune-compromised subjects.

In addition, the pharmaceutical composition of the present invention may contain one or more types of known active ingredients having an effect of preventing or treating an infectious disease caused by a biofilm along with the 4-gingerol derivative compound represented by Formula 1 or 2 or a racemate or isomer thereof.

In addition, the composition of the present invention may further include one or more types of pharmaceutically acceptable carriers, in addition to the active ingredient described above, for administration.

The pharmaceutically acceptable carrier may be saline, distilled water, Ringer's solution, buffered saline, a dextrose solution, a maltodextrin solution, glycerol, ethanol and a mixture of one or more thereof, and if needed, may further include other conventional additives such as an antioxidant, a buffered solution and an antibacterial agent. In addition, by additionally adding a diluent, a dispersant, a surfactant, a binder or a lubricant, the pharmaceutical composition may be formulated as an injectable form such as an aqueous solution, an emulsion or a suspension, a pill, a capsule, a granule or a tablet. Moreover, the pharmaceutically acceptable carrier is preferably prepared according to each disease or component by a suitable method in the art, or using a method disclosed in Remington's Pharmaceutical Science (Mack Publishing Company. Easton Pa.).

The composition of the present invention may be administered orally or parenterally according to a desired method, and a dose range may vary according to a patient's weight, age and sex, a health condition, a diet, administration time, an administration method, an excretion rate and the severity of a disease. The daily dose of the 4-gingerol derivative compound represented by Formula 1 or 2 or a racemate or isomer thereof may be 0.5 to 30 mg/kg, and preferably, approximately 10 to 20 mg/kg, and increased or decreased depending on a clinical trial result, and is preferably administered once or in multiple divided portions.

The term “subject” used herein may be a mammal such as a rat, livestock, a mouse or a human, and preferably, a human.

The composition of the present invention may be used independently for an effect of preventing or treating an infectious disease caused by a biofilm, or used in combination with a conventional disinfectant or antibiotic, hormonal treatment, drug treatment or a biological reaction modulator.

The 4-gingerol derivative compound according to the present invention or a racemate or isomer thereof may be prepared by a synthesis process as described below, and specifically, the following Scheme 1 schematically illustrates a process of synthesizing Compounds 11 and 12 by modifying a phenyl ring, the following Scheme 2 schematically illustrates a process of synthesizing Compounds 17 to 27 by modifying rotational rigidity and an absolute configuration, and the following Scheme 3 schematically illustrates a process of synthesizing Compounds 28 to 35.

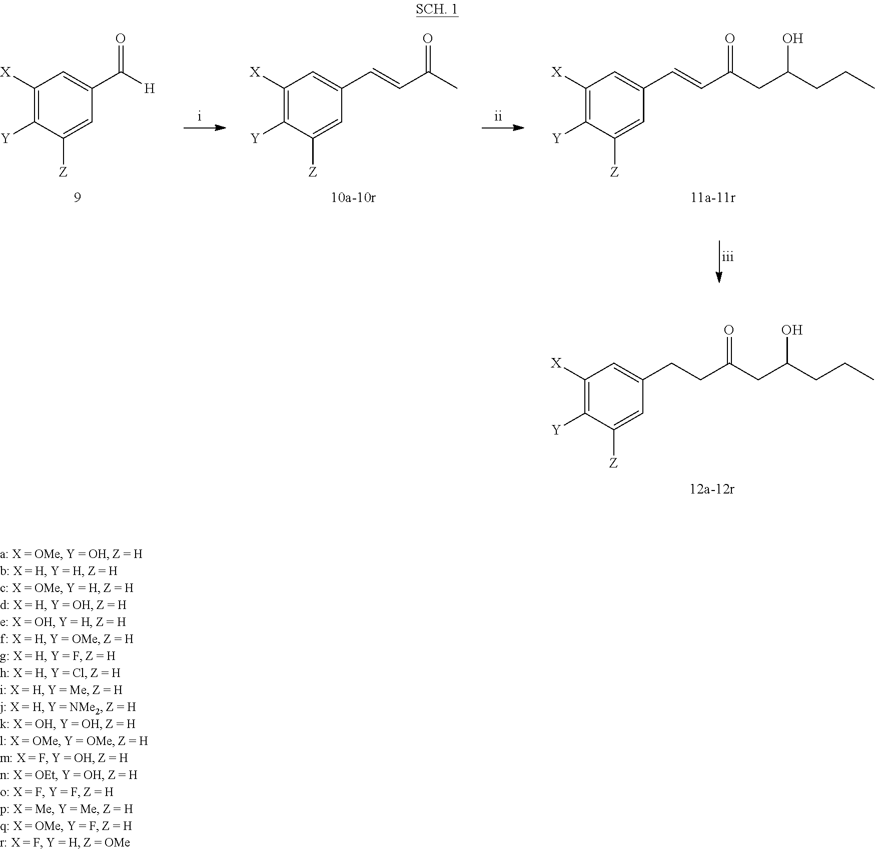

Conditions and samples for each reaction are as follows: (i) Acetone, ethanol (or water), 10% NaOH, room temperature, 2 to 24 hours, 32%; (ii) 1.0 M LDA in THF/hexanes, n-butanal, THF, −78° C. 2 to 12 hours, 24 to 60%; (iii) H₂ gas, 10% Pd/C, MeOH, room temperature, 2 hours, 43 to 93%.

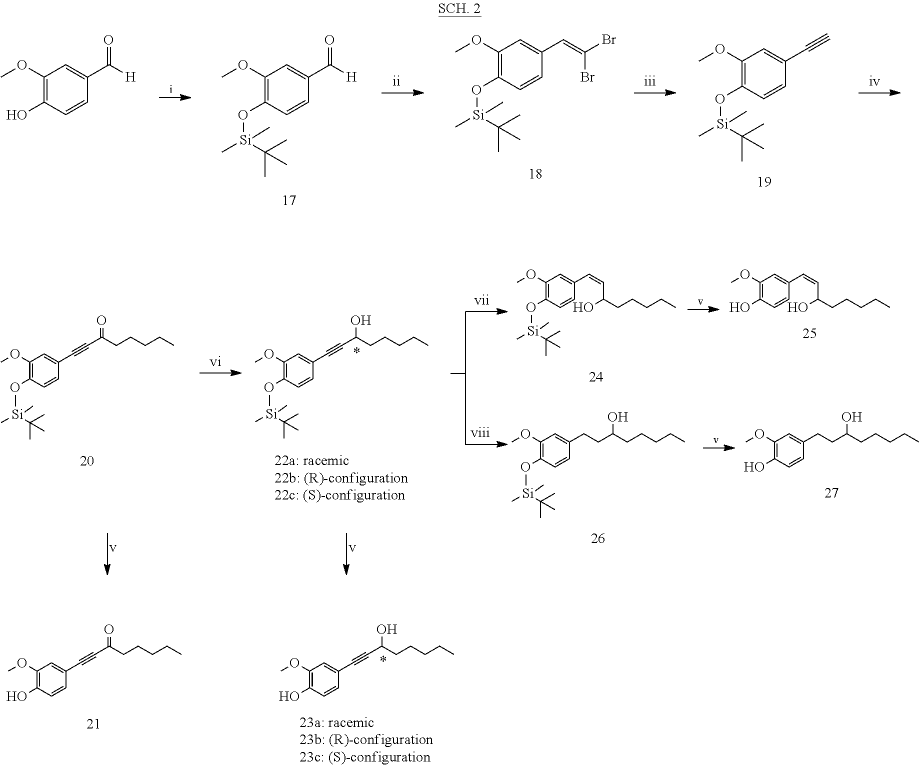

Conditions and samples for each reaction are as follows: (i) tert-butyldimethylsilyl chloride, imidazole, CH₂Cl₂, room temperature, 18 hours, 94%; (ii) PPh₃, CBr₄, CH₂Cl₂, room temperature, 3 hours, 98%, (iii) 1.6 M n-BuLi in hexanes (3.0 eq.), THF, room temperature, 2 hours, 96%; (iv) 1.6 M n-BuLi in hexanes (1.1 eq.), N-methoxy-N-methylhexanamide, THF, −78° C. to room temperature, 16 hours, 75%; (v) 0.1 M n-Bu₄NF in THF, room temperature, 1 hour, 94% (Compound 21), 94% (Compound 23a), 94% (Compound 23b), 93% (Compound 23c), 70% (Compound 25), and 94% (Compound 27); (vi) NaBH₄, MeOH, room temperature, 1 hour, 77% (Compound 22a), RuCl[(R, R)-TsDPEN(mesitylene)], KOH, 2-propanol, 4 hours, 88% (Compound 22b), and RuCl[(S, S)-TsDPEN(mesitylene)], KOH, 2-propanol, 4 hours, 68% (Compound 22c); (vii) Lindlar catalyst, H₂ gas, 1,4-benzoquinone, MeOH, 0° C., 1 hour (Compound 22a); (viii) H₂ gas, 10% Pd/C, MeOH, room temperature, 2 hours, 94% (Compound 22a).

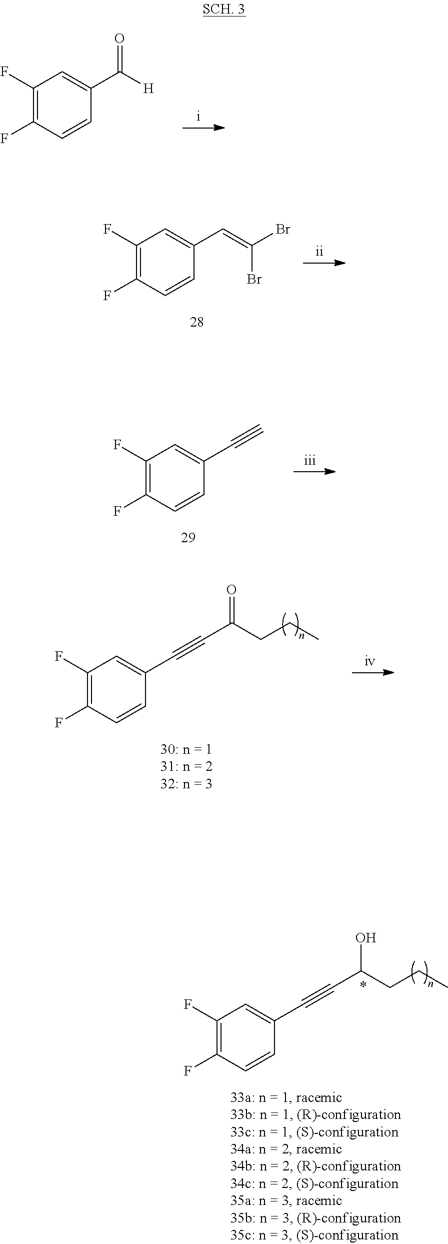

Conditions and samples for each reaction are as follows: (i) PPh₃, CBr₄, CH₂Cl₂, room temperature, 3 hours, 97%; (ii) 1.6 M n-BuLi in hexanes (3.0 eq.), THF, room temperature, 2 hours; (iii) 1.6 M n-BuLi in hexanes (1.1 eq.), suitable Weinreb amides, THF, −78° C. to room temperature, 16 hours, 72% (Compound 30), 88% (Compound 31), and 72% (Compound 32); (iv) NaBH₄, MeOH, room temperature, 1 hour (Compounds 33a to 35a), 88% (Compound 33a), 80% (Compound 34a), and 86% (Compound 35a), RuCl[(R, R)-TsDPEN(mesitylene)], KOH, 2-propanol, 4 hours (Compounds 33b to 35b), 86% (Compound 33b), 82% (Compound 34b), and 80% (Compound 35b), and RuCl[(S, S)-TsDPEN(mesitylene)], KOH, 2-propanol, 4 hours (Compounds 33c to 35c), 86% (Compound 33c), 82% (Compound 34c), and 82% (Compound 35c).

As the present invention may have various modifications and embodiments, specific embodiments of the present invention will be described in further detail below. However, the present invention is not limited to the specific embodiments, and it should be understood that the present invention includes all modifications, equivalents and alternatives included in the technical idea and scope of the present invention. To describe the present invention, when it is determined that a detailed description of the related art may obscure the gist of the present invention, the detailed description thereof will be omitted.

### EXPERIMENTAL METHOD

### Experimental Materials and General Experimental Details

All chemicals and solvents used in a reaction were purchased from Sigma-Aldrich, TCI, and Acros and used without purification. The reaction was monitored using TLC of a pre-coated silica gel plate (60F254) from Merck (Darmstadt, Germany). For detection, visualization was performed using an UV254 lamp and/or KMnO₄ staining. Column chromatography was performed on a silica gel (Silica gel 60; 230-400 mesh ASTM, Merck, Darmstadt, Germany). Nuclear magnetic resonance (NMR) spectra were recorded using Bruker BioSpin Avance 300 MHz NMR (¹H, 300 MHz; ¹³C, 75 MHz) or Bruker Ultrashield 600 MHz Plus (¹H, 600 MHz; ¹³C, 150 MHz) at room temperature. All chemical shifts were recorded in units of parts per million (ppm) using tetramethylsilane (TMS) as an internal standard, and relatively analyzed according to a solvent for a sample (CDCl₃: δ 7.26 for ¹H NMR, δ 77.0 for ¹³C NMR; MeOH-d₄: δ 3.31 for ¹H NMR, δ 49.0 for ¹³C NMR). A ¹H NMR shift value was expressed as a chemical shift (S), a corresponding integral, multiplicity, and coupling constant (J, Hz). High-resolution mass spectra (HRMS) were measured by Agilent 6530 Accurate Mass Q-TOF LC/MS. The purities of all of the final compounds were assessed using the Agilent 1260 Infinity (Agilent) with a C18 column (Phenomenex, 150 mm×4.6 mm, 3 μm, 110 Å) and measured by reverse phase-HPLC. RP-HPLC was performed using the following solvent conditions. In the case of Method A, mobile phases were acetonitrile and water (30:70, v/v), in the case of Method B, mobile phases were acetonitrile and 0.1% TFA-added water (30:70, v/v), in the case of Method C, mobile phases were acetonitrile and water (40:60, v/v), in the case of Method D, mobile phases were acetonitrile and water (50:50, v/v), in the case of Method E, mobile phases were acetonitrile and water (55:45, v/v), in the case of Method F, mobile phases were acetonitrile and water (60:40, v/v), in the case of Method G, mobile phases were methanol and water (50:50, v/v), in the case of Method H, mobile phases were methanol and water (60:40, v/v), and in the case of Method I, mobile phases were methanol and water (70:30, v/v). All compounds were eluted at a flow rate of 1 mL/min and monitored by a detector (wavelengths of 220 nm and 254 nm). The purities of the tested compounds were 95% or more.

### Synthesis of Compounds

Compounds 2 to 8

The following Compounds 2 to 8 were synthesized by a method disclosed in a prior patent document No. KR 10-2019-0010436.

TABLE 1

| | | |
|---|---|---|
| 4-gingerol | Compound 2 | 5-Hydroxy-1-(4-hydroxy-3- |
| | | methoxyphenyl)octan-3-one |
| 5-gingerol | Compound 3 | 5-Hydroxy-1-(4-hydroxy-3- |
| | | methoxyphenyl)nonan-3-one |
| 6-gingerol | Compound 4 | 5-Hydroxy-1-(4-hydroxy-3- |
| | | methoxyphenyl)decan-3-one |
| 7-gingerol | Compound 5 | 5-Hydroxy-1-(4-hydroxy-3- |
| | | methoxyphenyl)undecan-3-one |
| 8-gingerol | Compound 6 | 5-Hydroxy-1-(4-hydroxy-3- |
| | | methoxyphenyl)dodecan-3-one |
| 9-gingerol | Compound 7 | 5-Hydroxy-1-(4-hydroxy-3- |
| | | methoxyphenyl)tridecan-3-one |
| 10-gingerol | Compound 8 | 5-Hydroxy-1-(4-hydroxy-3- |
| | | methoxyphenyl)tetradecan-3-one |

### General Preparation Method A for Compounds 11a to 11r

A solution of a benzylideneacetone compound stirred in THF (20 mL) was added to LDA (1.2-3.6 eq.) at −78° C. The solution was stirred under argon (Ar) at the same temperature for 1 hour, and n-butanal (10.0 eq.) was slowly added dropwise using a syringe. After a TLC analysis result showed a complete conversion from a starting material to a product (generally, for 3 to 24 hours), the reaction mixture was vigorously stirred at the same temperature, quenched with an aqueous NH₄Cl solution (10 mL), and extracted with EtOAc (3×25 mL). An organic layer was washed with brine, dried with MgSO₄, filtered and concentrated under reduced pressure. The residue was purified by silica gel column chromatography to give a pure compound.

### (E)-5-Hydroxy-1-(4-hydroxy-3-methoxyphenyl)oct-1-en-3-one (11a)

Compound 11a was prepared as a yellow oil with (E)-4-(4-hydroxy-3-methoxyphenyl)but-3-en-2-one (300 mg, 1.56 mmol) and LDA (4.10 mL, 2.4 eq.) in 60% yield as described above in General Preparation Method A, and stirred for 3 hours. The residue was purified by silica gel column chromatography (Toluene/EtOAc=6:1 to 3:1, v/v). R_{f}=0.40 (Toluene/EtOAc=2:1, v/v). ¹H NMR (300 MHz, CDCl₃) δ 7.53 (d, J=16.1 Hz, 1H), 7.13 (dd, J=1.8 and 8.2 Hz, 1H), 7.08 (d, J=1.7 Hz, 1H), 6.96 (d, J=8.2 Hz, 1H), 6.61 (d, J=16.1 Hz, 1H), 5.94 (s, 1H), 4.23-4.09 (m, 1H), 3.96 (s, 3H), 3.30 (d, J=2.9 Hz, 1H), 2.90 (dd, J=2.7 and 17.1 Hz, 1H), 2.75 (q, J=9.0 Hz, 1H), 1.59-1.36 (m, 4H), 0.97 (t, J=7.2 Hz, 3H); ¹³C NMR (75 MHz, CDCl₃) δ 201.03, 148.50, 146.89, 143.87, 126.72, 124.15, 123.76, 114.87, 109.51, 67.65, 55.98, 46.52, 38.68, 18.75, 14.05, 13.97. HRMS m/z calculated for C₁₅H₂₀O₄[M-H]⁻: 263.1289. found: 263.1304. >95% purity (as determined by RP-HPLC, method C, t_{R}=6.34 min, method G, t_{R}=7.33 min).

### (E)-5-Hydroxy-1-phenyloct-1-en-3-one (11b)

Compound 11b was prepared as a yellow oil with (E)-4-phenylbut-3-en-2-one (300 mg, 2.05 mmol) and LDA (2.70 mL, 1.2 eq.) in 36% yield as described in General Preparation Method A, and stirred for 2 hours. The residue was purified by silica gel column chromatography (Toluene/EtOAc=6:1 to 3:1, v/v). R_{f}=0.40 (Toluene/EtOAc=2:1, v/v). ¹H NMR (600 MHz, CDCl₃) δ 7.58 (d, J=16.2 Hz, 1H), 7.56-7.54 (m, 2H), 7.43-7.39 (m, 3H), 6.74 (d, J=16.2 Hz, 1H), 4.19-4.13 (m, 1H), 3.17 (d, J=3.30 Hz, 1H), 2.88 (dd, J=2.6 and 17.3 Hz, 1H), 2.77 (q, J=9.1 Hz, 1H), 1.62-1.36 (m, 4H), 0.96 (t, J=7.1 Hz, 3H); ¹³C NMR (150 MHz, CDCl₃) δ 211.24, 140.75, 128.56, 128.29, 126.22, 67.36, 49.32, 45.06, 38.59, 29.53, 18.65, 13.98. HRMS m/z calculated for C₁₄H₁₈O₂ [M-H]⁻: 217.1234. found: 217.1252. >95% purity (as determined by RP-HPLC, method C, t_{R}=12.91 min, method G, t_{R}=15.79 min).

### (E)-5-Hydroxy-1-(3-methoxyphenyl)oct-1-en-3-one (11c)

Compound 11c was prepared as a yellow oil with (E)-4-(3-methoxyphenyl)but-3-en-2-one (300 mg, 1.70 mmol) and LDA (2.23 mL, 1.2 eq.) in 41% yield as described in General Preparation Method A, and stirred for 4 hours. The residue was purified by silica gel column chromatography (Hexane/EtOAc=10:1 to 6:1, v/v). R_{f}=0.48 (Hexane/EtOAc=2:1, v/v). ¹H NMR (600 MHz, CDCl₃) δ 7.55 (d, J=16.1 Hz, 1H), 7.33 (t, J=7.9 Hz, 1H), 7.16 (d, J=7.6 Hz, 1H), 7.08 (s, 1H), 6.98 (d, J=8.2 Hz, 1H), 6.73 (d, J=16.2 Hz, 1H), 4.17 (brs, 1H), 3.86 (s, 3H), 3.22 (brs, 1H), 2.91-2.88 (m, 1H), 2.80-2.76 (m, 1H), 1.61-1.40 (m, 4H), 0.97 (t, J=7.1 Hz, 3H); ¹³C NMR (150 MHz, CDCl₃) δ 201.04, 159.98, 143.48, 135.60, 130.02, 126.65, 121.15, 116.66, 113.20, 67.61, 55.35, 46.86, 38.69, 18.75, 14.04. HRMS m/z calculated for C₁₅H₂₀O₃ [M-H]⁻: 247.1339. found: 247.1344. >95% purity (as determined by RP-HPLC, method C, t_{R}=14.25 min, method G, t_{R}=19.69 min).

### (E)-5-Hydroxy-1-(4-hydroxyphenyl)oct-1-en-3-one (11d)

Compound 11d was prepared as a yellow oil with (E)-4-(4-hydroxyphenyl)but-3-en-2-one (250 mg, 1.54 mmol) and LDA (4.10 mL, 2.4 eq.) in 43% yield as described in General Preparation Method A, and stirred for 6 hours. The residue was purified by silica gel column chromatography (Hexane/EtOAc=10:1 to 3:1, v/v). R_{f}=0.42 (Hexane/EtOAc=2:1, v/v). ¹H NMR (600 MHz, MeOD) δ 7.61 (d, J=16.1 Hz, 1H), 7.53 (d, J=8.6 Hz, 1H), 6.84 (d, J=8.6 Hz, 1H), 6.72 (d, J=16.1 Hz, 1H), 4.14-4.12 (m, 1H), 2.88-2.84 (m, 1H), 2.78-2.75 (m, 1H), 1.55-1.41 (m, 4H), 0.97 (t, J=7.1 Hz, 3H); ¹³C NMR (150 MHz, MeOD) δ 200.62, 160.22, 144.09, 130.21, 125.86, 123.16, 115.54, 67.81, 39.25, 18.45, 12.98. HRMS m/z calculated for C₁₄H₁₈O₃ [M-H]⁻: 233.1183. found: 233.1213. >95% purity (as determined by RP-HPLC, method C, t_{R}=6.18 min, method G, t_{R}=8.08 min).

### (E)-5-Hydroxy-1-(3-hydroxyphenyl)oct-1-en-3-one (11e)

Compound 11e was prepared as a yellow oil with (E)-4-(3-hydroxyphenyl)but-3-en-2-one (250 mg, 1.54 mmol) and LDA (4.10 mL, 2.4 eq.) in 34% yield as described in General Preparation Method A, and stirred for 6 hours. The residue was purified by silica gel column chromatography (Hexane/EtOAc=10:1 to 3:1, v/v). R_{f}=0.30 (Hexane/EtOAc=2:1, v/v). ¹H NMR (600 MHz, MeOD) δ 7.58 (d, J=16.1 Hz, 1H), 7.24 (t, J=7.9 Hz, 1H), 7.11 (d, J=7.6 Hz, 1H), 7.06 (s, 1H), 6.87 (dd, J=8.1 and 1.8 Hz, 1H), 6.80 (d, J=16.2 Hz, 1H), 4.16-4.11 (m, 1H), 2.90-2.86 (m, 1H), 2.79-2.77 (m, 1H), 1.54-1.40 (m, 4H), 0.97 (t, J=7.1 Hz, 3H); ¹³C NMR (150 MHz, MeOD) δ 200.49, 157.73, 143.65, 135.85, 129.65, 126.18, 119.78, 117.53, 114.14, 67.66, 39.25, 18.45, 12.96. HRMS m/z calculated for C₁₄H₁₈O₃ [M-H]⁻: 233.1183. found: 233.1200. >95% purity (as determined by RP-HPLC, method C, t_{R}=5.68 min, method G, t_{R}=8.17 min).

### (E)-5-Hydroxy-1-(4-methoxyphenyl)oct-1-en-3-one (11f)

Compound 11f was prepared as a white oil with (E)-4-(4-methoxyphenyl)but-3-en-2-one (300 mg, 1.70 mmol) and LDA (2.30 mL, 1.2 eq.) in 48% yield as described in General Preparation Method A, and stirred for 2 hours. The residue was purified by silica gel column chromatography (Toluene/EtOAc=10:1 to 3:1, v/v). R_{f}=0.55 (Toluene/EtOAc=2:1, v/v). ¹H NMR (600 MHz, CDCl₃) δ 7.55-7.50 (m, 3H), 6.93 (d, J=8.7, 2H), 6.62 (d, J=16.2 Hz, 1H), 4.14 (brs, 1H), 3.85 (s, 3H), 3.27 (brs, 1H), 2.88-2.85 (m, 1H), 2.75-2.71 (m, 1H), 1.62-1.42 (m, 4H), 0.95 (t, J=7.2 Hz, 3H); ¹¹C NMR (150 MHz, CDCl₃) δ 201.01, 161.83, 143.39, 130.19, 126.87, 124.18, 114.49, 67.73, 55.41, 46.71, 38.75, 18.75, 14.04. HRMS m/z calculated for C₁₅H₂NO₃ [M-H]⁻: 247.1339. found: 247.1357. >95% purity (as determined by RP-HPLC, method C, t_{R}=12.88 min, method G, t_{R}=18.64 min).

### (E)-1-(4-Fluorophenyl)-5-hydroxyoct-1-en-3-one (11e)

Compound 11g was prepared as a yellow oil with (E)-4-(4-fluorophenyl)but-3-en-2-one (200 mg, 1.22 mmol) and LDA (1.60 mL, 1.2 eq.) in 38% yield as described in General Preparation Method A, and stirred for 2 hours. The residue was purified by silica gel column chromatography (Hexane/EtOAc=10:1 to 6:1, v/v). R_{f}=0.40 (Hexane/EtOAc=2:1, v/v). ¹H NMR (600 MHz, CDCl₃) δ 7.56-7.53 (m, 3H), 7.10 (t, J=8.6 Hz, 2H), 6.66 (d, J=16.2 Hz), 4.17 (brs, 1H), 3.13 (s, 1H), 2.88-2.85 (m, 1H), 2.77-2.73 (m, 1H), 1.59-1.40 (m, 4H), 0.96 (t, J=7.1 Hz, 3H); ¹³C NMR (150 MHz, CDCl₃) δ 200.74, 165.03, 163.36, 142.15, 130.50, 130.48, 130.35, 130.30, 126.08, 126.07, 116.30, 116.16, 67.61, 47.04, 38.70, 18.74, 14.02. HRMS m/z calculated for C₁₄H₁₇FO₂ [M-H]⁻: 235.1140. found: 235.1155. >95% purity (as determined by RP-HPLC, method C, t_{R}=14.12 min, method G, t_{R}=17.64 min).

### (E)-1-(4-Chlorophenyl)-5-hydroxyoct-1-en-3-one (11h)

Compound 11h was prepared as a yellow oil with (E)-4-(4-(dimethylamino)phenyl)but-3-en-2-one (200 mg, 1.11 mmol) and LDA (1.40 mL, 1.2 eq.) in 41% yield as described in General Preparation Method A, and stirred for 6 hours. The residue was purified by silica gel column chromatography (Hexane/EtOAc=12:1 to 6:1, v/v). R_{f}=0.35 (Hexane/EtOAc=2:1, v/v). ¹H NMR (600 MHz, CDCl₃) δ 7.52 (d, J=16.2 Hz, 1H), 7.48 (d, J=8.8 Hz, 2H), 7.37 (d, J=8.5 Hz, 2H), 6.70 (d, J=16.2 Hz, 1H), 4.15 (brs, 1H), 3.13 (brs, 1H), 2.87-2.84 (m, 1H), 2.78-2.74 (m, 1H), 1.59-1.39 (m, 4H), 0.95 (t, J=7.2 Hz, 3H); ¹³C NMR (150 MHz, CDCl₃) δ 200.64, 141.91, 136.66, 132.75, 129.54, 129.28, 126.72, 67.58, 47.21, 38.75, 18.75, 14.02. HRMS m/z calculated for C₁₄H₁₇ClO₂ [M-H]⁻: 251.0844. found: 251.0862. >95% purity (as determined by RP-HPLC, method C, t_{R}=22.36 min, method G, t_{R}=34.88 min).

### (E)-5-Hydroxy-1-(o-tolyl)oct-1-en-3-one (11i)

Compound 11i was prepared as a yellow oil with (E)-4-(p-tolyl)but-3-en-2-one (300 mg, 1.87 mmol) and LDA (2.48 mL, 1.2 eq.) in 29% yield as described in General Preparation Method A, and stirred for 4 hours. The residue was purified by silica gel column chromatography (Hexane/EtOAc=8:1 to 4:1, v/v). R_{f}=0.45 (Hexane/EtOAc=2:1, v/v). ¹H NMR (600 MHz, CDCl₃) δ 7.55 (d, J=16.2 Hz, 1H), 7.45 (d, J=8.2 Hz, 2H), 7.21 (d, J=8.0 Hz, 2H), 6.69 (d, J=16.2 Hz, 1H), 4.16-4.14 (m, 1H), 3.25 (d, J=3.2 Hz, 1H), 2.88-2.85 (m, 1H), 2.77-2.72 (m, 1H), 2.38 (s, 1H), 1.59-1.39 (m, 4H), 0.95 (t, J=7.1 Hz, 3H); ¹³C NMR (150 MHz, CDCl₃) δ 201.10, 143.63, 141.37, 131.48, 129.77, 128.44, 125.46, 46.77, 38.72, 21.53, 18.75, 14.04. HRMS m/z calculated for C₁₅H₂₀O₂ [M-H]⁻: 231.1390. found: 231.1404. >95% purity (as determined by RP-HPLC, method C, t_{R}=19.34 min, method G, t_{R}=30.62 min).

### (E)-1-(4-(Dimethylamino)phenyl)-5-hydroxyoct-1-en-3-one (11j)

Compound 11j was prepared as a yellow oil with (E)-4-(4-(dimethylamino)phenyl)but-3-en-2-one (200 mg, 1.06 mmol) and LDA (1.40 mL, 1.2 eq.) in 60% yield as described in General Preparation Method A, and stirred for 8 hours. The residue was purified by silica gel column chromatography (Hexane/EtOAc=10:1 to 3:1, v/v). R_{f}=0.31 (Hexane/EtOAc=2:1, v/v). ¹H NMR (600 MHz, CDCl₃) δ 7.52 (d, J=16.0 Hz, 1H), 7.45 (d, J=8.9 Hz, 2H), 6.67 (d, J=8.9 Hz, 2H), 6.54 (d, J=16.0 Hz, 1H), 4.13-4.11 (m, 1H), 3.50-3.49 (m, 1H), 3.04 (s, 6H), 2.88-2.84 (m, 1H), 2.71-2.67 (m, 1H), 1.60-1.39 (m, 4H), 0.95 (t, J=7.2 Hz, 3H); ¹³C NMR (150 MHz, CDCl₃) δ 201.06, 152.15, 144.52, 130.34, 121.75, 121.40, 111.84, 67.90, 46.22, 40.10, 38.78, 18.77, 14.08. HRMS m/z calculated for C₁₆H₂₃NO₂ [M-H]⁻: 260.1656. found: 260.1674. >95% purity (as determined by RP-HPLC, method C, t_{R}=17.13 min, method G, t_{R}=30.68 min).

### (E)-1-(3,4-Dihydroxyphenyl)-5-hydroxyoct-1-en-3-one (11k)

Compound 11k was prepared as a yellow oil with (E)-4-(3,4-dihydroxyphenyl)but-3-en-2-one (150 mg, 0.84 mmol) and LDA (3.30 mL, 3.6 eq.) in 30% yield as described in General Preparation Method A, and stirred for 6 hours. The residue was purified by silica gel column chromatography (Hexane/EtOAc=3:1 to 1:1, v/v). R_{f}=0.50 (Hexane/EtOAc=1:3, v/v). ¹H NMR (600 MHz, MeOD) δ 7.53 (d, J=16.1 Hz, 1H), 7.10 (d, J=1.8 Hz, 1H), 7.01 (dd, J=1.8 and 8.2 Hz, 1H), 6.81 (d, J=8.2 Hz, 1H), 6.64 (d, J=16.1 Hz, 1H), 4.18-4.05 (m, 1H), 2.84 (q, J=8.2 Hz, 1H), 2.74 (dd, J=4.4 and 15.4 Hz, 1H), 1.57-1.35 (m, 4H), 0.96 (t, J=7.2 Hz, 3H); ¹³C NMR (150 MHz, MeOD) δ 200.69, 148.58, 145.48, 144.61, 126.45, 123.18, 122.21, 115.20, 114.03, 67.86, 39.23, 18.47, 13.00. HRMS m/z calculated for C₁₄H₁₈O₄ [M-H]⁻: 249.1132. found: 249.1232. >95% purity (as determined by RP-HPLC, method B, t_{R}=5.21 min, method G, t_{R}=5.60 min).

### (E)-1-(3,4-Dimethoxyphenyl)-5-hydroxyoct-1-en-3-one (11l)

Compound 11l was prepared as a yellow oil with (E)-4-(3,4-dimethoxyphenyl)but-3-en-2-one (200 mg, 0.97 mmol) and LDA (1.30 mL, 1.2 eq.) in 54% yield as described in General Preparation Method A, and stirred for 1 hour. The residue was purified by silica gel column chromatography (Toluene/EtOAc=10:1 to 3:1, v/v). R_{f}=0.50 (Toluene/EtOAc=2:1, v/v). ¹H NMR (600 MHz, CDCl₃) δ 7.53 (d, J=16.1 Hz, 1H), 7.14 (d, J=8.3 Hz, 1H), 7.07 (s, 1H), 6.89 (d, J=8.3 Hz, 1H), 6.61 (d, J=16.1 Hz, 1H), 4.15 (brs, 1H), 3.93 (s, 6H), 3.25 (s, 1H), 2.89-2.86 (m, 1H), 2.77-2.72 (m, 1H), 1.61-1.40 (m, 4H), 0.96 (t, J=7.1 Hz, 3H); ¹³C NMR (150 MHz, CDCl₃) δ 200.95, 151.60, 149.30, 143.66, 127.13, 124.41, 123.33, 111.10, 109.74, 67.72, 56.00, 55.91, 46.63, 38.73, 18.74, 14.04. HRMS m/z calculated for C₁₆H₂₂O₄[M-H]⁻: 277.1445. found: 277.1478. >95% purity (as determined by RP-HPLC, method C, t_{R}=8.92 min, method G, t_{R}=11.13 min).

### (E)-1-(3-Fluoro-4-hydroxyphenyl)-5-hydroxyoct-1-en-3-one (11m)

Compound 11m was prepared as a yellow oil with (E)-4-(3-fluoro-4-hydroxyphenyl)but-3-en-2-one (100 mg, 0.55 mmol) and LDA (1.50 mL, 2.4 eq.) in 45% yield as described in General Preparation Method A, and stirred for 4 hours. The residue was purified by silica gel column chromatography (Hexane/EtOAc=10:1 to 3:1, v/v). R_{f}=0.25 (Hexane/EtOAc=2:1, v/v). ¹H NMR (600 MHz, MeOD) δ 7.56 (d, J=16.1 Hz, 1H), 7.42 (d, J=12.1 Hz, 1H), 7.31 (d, J=8.4 Hz, 1H), 6.95 (t, J=8.6 Hz, 3H), 6.73 (d, J=16.1 Hz, 1H), 4.14 (brs, 1H), 3.26 (s, 1H), 2.88-2.83 (m, 1H), 2.78-2.75 (m, 1H), 1.53-1.40 (m, 4H), 0.96 (t, J=7.1 Hz, 3H); ¹³C NMR (150 MHz, MeOD) δ 200.36, 152.43, 150.82, 147.75, 147.66, 142.75, 142.74, 126.70, 126.66, 125.76, 125.74, 124.49, 117.67, 117.65, 115.12, 114.99, 67.73, 39.25, 18.46, 12.99. HRMS m/z calculated for C₁₄H₁₇FO₃ [M-H]⁻: 251.1089. found: 251.1117. >95% purity (as determined by RP-HPLC, method C, t_{R}=6.49 min, method G, t_{R}=8.91 min).

### (E)-1-(3-Ethoxy-4-hydroxyphenyl)-5-hydroxyoct-1-en-3-one (11n)

Compound 11n was prepared as a yellow oil with (E)-4-(3-ethoxy-4-hydroxyphenyl)but-3-en-2-one (200 mg, 0.97 mmol) and LDA (2.60 mL, 2.4 eq.) in 39% yield as described in General Preparation Method A, and stirred for 3 hours. The residue was purified by silica gel column chromatography (Hexane/EtOAc=10:1 to 3:1, v/v). R_{f}=0.24 (Hexane/EtOAc=2:1, v/v). ¹H NMR (600 MHz, CDCl₃) δ 7.50 (d, J=16.1 Hz, 1H), 7.10 (d, J=8.1 Hz, 1H), 7.04 (s, 1H), 6.94 (d, J=8.2 Hz, 1H), 6.58 (d, J=16.1 Hz, 1H), 5.94 (s, 1H), 4.18-4.15 (q, 7.0 Hz, 2H), 3.26 (s, 1H), 2.88-2.85 (m, 1H), 2.75-2.71 (m, 1H), 1.50-1.47 (m, 4H), 0.95 (t, J=7.1 Hz, 3H); ¹³C NMR (150 MHz, CDCl₃) δ 201.02, 148.69, 146.22, 143.98, 126.64, 124.05, 123.59, 114.89, 110.44, 67.76, 64.64, 46.52, 38.70, 18.75, 14.79, 14.05. HRMS m/z calculated for C₁₆H₂₂O₄ [M-H]⁻: 277.1445. found: 277.1472. >95% purity (as determined by RP-HPLC, method C, t_{R}=8.42 min, method G, t_{R}=10.65 min).

### (E)-1-(3,4-Difluorophenyl)-5-hydroxyoct-1-en-3-one (11o)

Compound 11o was prepared as a yellow oil with (E)-4-(3,4-difluorophenyl)but-3-en-2-one (200 mg, 1.10 mmol) and LDA (1.60 mL, 1.2 eq.) in 38% yield as described in General Preparation Method A, and stirred for 18 hours. The residue was purified by silica gel column chromatography (Hexane/EtOAc=10:1 to 2:1, v/v). R_{f}=0.38 (Hexane/EtOAc=2:1, v/v). ¹H NMR (600 MHz, CDCl₃) δ 7.47 (d, J=16.1 Hz, 1H), 7.38 (t, J=9.2 Hz, 1H), 7.30-7.28 (m, 1H), 7.20 (q, J=8.7 Hz, 1H), 6.64 (d, J=16.1 Hz, 1H), 4.16 (brs, 1H), 3.09 (s, 1H), 2.86-2.83 (m, 1H), 2.78-2.74 (m, 1H), 1.60-1.36 (m, 4H), 0.96 (t, J=7.1 Hz, 3H); ¹³C NMR (150 MHz, CDCl₃) δ 200.32, 152.61, 152.52, 151.52, 151.43, 150.92, 150.84, 149.87, 149.78, 140.88, 131.56, 131.52, 131.49, 127.09, 127.08, 125.26, 125.23, 125.21, 125.19, 118.01, 117.89, 116.58, 116.47. HRMS m/z calculated for C₁₄H₁₆F₂O₂ [M-H]⁻: 253.1045. found: 253.1062. >95% purity (as determined by RP-HPLC, method C, t_{R}=17.12 min, method G, t_{R}=22.40 min).

### (E)-1-(3,4-Dimethylphenyl)-5-hydroxyoct-1-en-3-one (11p)

Compound 11p was prepared as a yellow oil with (E)-4-(3,4-dimethylphenyl)but-3-en-2-one (200 mg, 1.15 mmol) and LDA (1.50 mL, 1.2 eq.) in 37% yield as described in General Preparation Method A, and stirred for 8 hours. The residue was purified by silica gel column chromatography (Hexane/EtOAc=8:1 to 4:1, v/v). R_{f}=0.55 (Hexane/EtOAc=2:1, v/v). ¹H NMR (600 MHz, CDCl₃) δ 7.53 (d, J=16.1 Hz, 1H), 7.32 (s, 1H), 7.29 (d, J=7.8 Hz, 1H), 7.16 (d, J=7.7 Hz, 1H), 6.68 (d, J=16.2 Hz, 1H), 4.16-4.12 (m, 1H), 3.29 (brs, 1H), 2.88-2.85 (m, 1H), 2.76-2.72 (m, 1H), 2.28 (s, 6H), 1.60-1.39 (m, 4H), 0.95 (t, J=7.1 Hz, 3H); ¹³C NMR (150 MHz, CDCl₃) δ 201.12, 143.86, 140.14, 137.30, 131.87, 130.30, 129.63, 126.10, 125.31, 67.67, 46.73, 38.73, 19.88, 19.75, 18.76, 14.05. HRMS m/z calculated for C₁₆H₂₂O₂ [M-H]⁻: 245.1547. found: 245.1558. >95% purity (as determined by RP-HPLC, method C, t_{R}=28.39 min, method H, t_{R}=19.67 min).

### (E)-1-(4-Fluoro-3-methoxyphenyl)-5-hydroxyoct-1-en-3-one (11q)

Compound 11q was prepared as a white oil with (E)-4-(4-fluoro-3-methoxyphenyl)but-3-en-2-one (120 mg, 0.62 mmol) and LDA (0.70 mL, 1.2 eq.) in 46% yield as described in General Preparation Method A, and stirred for 2 hours. The residue was purified by silica gel column chromatography (Hexane/EtOAc=10:1 to 3:1, v/v). R_{f}=0.48 (Hexane/EtOAc=2:1, v/v). ¹H NMR (600 MHz, CDCl₃) δ 7.51 (d, J=16.1 Hz, 1H), 7.14 (d, J=8.0 Hz, 1H), 7.12-7.08 (m, 2H), 6.64 (d, J=16.1 Hz, 1H), 4.17-4.14 (m, 1H), 3.93 (s, 3H), 3.15 (brs, 1H), 2.88-2.85 (m, 1H), 2.78-2.74 (m, 1H), 1.59-1.40 (m, 4H), 0.96 (t, J=7.2 Hz, 3H); ¹³C NMR (150 MHz, CDCl₃) δ 200.74, 154.87, 153.19, 148.18, 148.10, 142.57, 130.89, 130.87, 126.18, 126.16, 122.10, 122.06, 116.67, 116.55, 112.46, 67.62, 56.27, 46.95, 38.71, 18.75, 14.03. HRMS m/z calculated for C₁₅H₁₉FO₃ [M-H]⁻: 265.1245. found: 265.1496. >95% purity (as determined by RP-HPLC, method C, t_{R}=15.14 min, method G, t_{R}=19.00 min).

### (E)-1-(3-Fluoro-5-methoxyphenyl)-5-hydroxyoct-1-en-3-one (11r)

Compound 11r was prepared as a yellow oil with (E)-4-(3-fluoro-5-methoxyphenyl)but-3-en-2-one (130 mg, 0.67 mmol) and LDA (0.88 mL, 1.2 eq.) in 24% yield as described in General Preparation Method A, and stirred for 6 hours. The residue was purified by silica gel column chromatography (Hexane/EtOAc=10:1 to 6:1, v/v). R_{f}=0.40 (Hexane/EtOAc=2:1, v/v). ¹H NMR (600 MHz, CDCl₃) δ 7.46 (d, J=16.2 Hz, 1H), 6.86-6.85 (m, 2H), 6.70-6.65 (m, 2H), 4.17-4.14 (m, 1H), 3.83 (s, 3H), 3.13 (brs, 1H), 2.87-2.84 (m, 1H), 2.79-2.74 (m, 1H), 1.59-1.40 (m, 4H), 0.95 (t, J=7.1 Hz, 3H); ¹³C NMR (150 MHz, CDCl₃) δ 200.68, 164.57, 162.93, 161.26, 161.18, 142.14, 142.12, 136.79, 136.72, 127.55, 110.12, 110.11, 107.09, 106.94, 103.86, 103.70, 67.56, 55.69, 47.21, 38.70, 18.74, 14.01. HRMS m/z calculated for C₁₅H₁₉FO₃ [M-H]⁻: 265.1245. found: 265.1262. >95% purity (as determined by RP-HPLC, method C, t_{R}=19.07 min, method H, t_{R}=11.43 min).

### General Preparation Method B for Compounds 12a to 12r

Each of the stirred Compound 11a-11r solutions in MeOH (8 mL) as a solvent was added to 10% Pd/C (0.03 eq.), and stirred in hydrogen gas for 2 hours. The reaction mixture was filtered through a Celite pad, and concentrated under reduced pressure. The residue was purified by silica gel column chromatography (Hexane/EtOAc=4:1, v/v), thereby obtaining each of Compounds 12a to 12r.

### 5-Hydroxy-1-(4-hydroxy-3-methoxyphenyl)octan-3-one (12a)

Compound 12a was prepared as a colorless oil with Compound 11a (109 mg, 0.412 mmol) in 45% yield as described in General preparation method B. R_{f}=0.38 (Hexane/EtOAc=1:1, v/v). ¹H NMR (300 MHz, CDCl₃) δ 6.82 (d, J=8.2 Hz, 1H), 6.73-6.60 (m, 2H), 5.71 (s, 1H), 4.04 (brs, 1H), 3.86 (s, 3H), 3.06 (brs, 1H), 2.90-2.65 (m, 4H), 2.62-2.41 (m, 2H), 1.56-1.21 (m, 4H), 0.91 (t, J=7.2 Hz, 3H); ¹³C NMR (75 MHz, CDCl₃) δ 211.53, 146.45, 143.93, 132.63, 120.74, 114.37, 111.02, 67.38, 67.27, 55.88, 49.34, 45.44, 38.54, 29.28, 18.65, 13.98. HRMS m/z calculated for C₁₅H₂₂O₃ [M-H]⁻: 265.1445. found: 265.1455. >95% purity (as determined by RP-HPLC, method C, t_{R}=6.09 min, method G, t_{R}=5.49 min).

### 5-Hydroxy-1-phenyloctan-3-one (12b)

Compound 12b was prepared as a colorless oil with Compound 11b (50 mg, 0.229 mmol) in 52% yield as described in General preparation method B. R_{f}=0.65 (Hexane/EtOAc=1:1, v/v). ¹H NMR (600 MHz, CDCl₃) δ 7.29-7.26 (m, 2H), 7.20-7.17 (m, 3H), 4.05-4.03 (m, 1H), 2.94 (s, 1H), 2.90 (t, J=7.6 Hz, 2H), 2.76 (t, J=7.6 Hz, 2H), 2.57-2.47 (m, 2H), 1.49-1.25 (m, 4H), 0.91 (t, J=7.1 Hz, 3H); ¹³C NMR (150 MHz, CDCl₃) δ 211.24, 140.75, 128.56, 128.29, 126.22, 67.36, 49.32, 45.06, 38.59, 29.53, 18.65, 13.98. HRMS m/z calculated for C₁₄H₂₀O₂[M-H]⁻: 219.1390; found: 219.1414. >95% purity (as determined by RP-HPLC, method C, t_{R}=14.38 min, method G, t_{R}=16.36 min).

### 5-Hydroxy-1-(3-methoxyphenyl)octan-3-one (12c)

Compound 12c was prepared as a colorless oil with Compound 11c (50 mg, 0.201 mmol) in 72% yield as described in General preparation method B. R_{f}=0.51 (Hexane/EtOAc=1:1, v/v). ¹H NMR (600 MHz, CDCl₃) δ 7.20 (t, J=7.9 Hz, 1H), 6.77-6.72 (m, 3H), 4.05-4.03 (m, 1H), 3.79 (s, 3H), 2.90 (d, J=3.4 Hz, 1H), 2.88 (t, J=7.6 Hz, 2H), 2.76 (t, J=7.6 Hz, 2H), 2.58-2.55 (m, 1H), 2.52-2.47 (m, 1H), 1.50-1.31 (m, 4H), 0.92 (t, J=7.1 Hz, 3H); ¹³C NMR (150 MHz, CDCl₃) δ 211.15, 159.75, 142.38, 129.54, 120.61, 114.13, 111.46, 67.36, 55.16, 49.33, 44.96, 38.61, 29.55, 18.65, 13.98. HRMS m/z calculated for C₁₅H₂₂O₃ [M-H]⁻: 250.1569. found: 249.1518. >95% purity (as determined by RP-HPLC, method C, t_{R}=13.80 min, method G, t_{R}=15.38 min).

### 5-Hydroxy-1-(4-hydroxyphenyl)octan-3-one (12d)

Compound 12d was prepared as a colorless oil with Compound 11d (88 mg, 0.376 mmol) in 72% yield as described in General preparation method B. R_{f}=0.45 (Hexane/EtOAc=1:1, v/v). ¹H NMR (600 MHz, MeOD) δ 7.02 (d, J=8.5 Hz, 1H), 6.70 (d, J=8.5 Hz, 1H), 4.04-4.01 (m, 1H), 2.77 (s, 4H), 2.58-2.54 (m, 1H), 2.52-2.48 (m, 1H), 1.46-1.33 (m, 4H), 0.93 (t, J=7.1 Hz, 3H); ¹³C NMR (150 MHz, MeOD) δ 210.62, 155.21, 131.87, 128.88, 114.78, 67.24, 49.89, 45.03, 39.19, 28.39, 18.37, 12.94. HRMS m/z calculated for C₁₄H₂₀O₃ [M-H]⁻: 235.1339. found: 235.1360. >95% purity (as determined by RP-HPLC, method C, t_{R}=6.02 min, method G, t_{R}=6.14 min).

### 5-Hydroxy-1-(3-hydroxyphenyl)octan-3-one (12e)

Compound 12e was prepared as a colorless oil with Compound 11e (50 mg, 0.213 mmol) in 68% yield as described in General preparation method B. R_{f}=0.35 (Hexane/EtOAc=1:1, v/v). ¹H NMR (600 MHz, MeOD) δ 7.07 (t, J=7.8 Hz, 1H), 6.67 (d, J=7.6 Hz, 1H), 6.64 (s, 1H), 6.61-6.60 (m, 1H), 4.05-4.02 (m, 1H), 2.80 (s, 4H), 2.59-2.55 (m, 1H), 2.53-2.50 (m, 1H), 1.48-1.31 (m, 4H), 0.94 (t, J=7.1 Hz, 3H); ¹³C NMR (150 MHz, MeOD) δ 210.35, 157.09, 142.67, 129.03, 119.16, 114.81, 112.55, 67.26, 49.87, 44.58, 39.20, 29.14, 18.37, 12.93. HRMS m/z calculated for C₁₄H₂₀O₃ [M-H]⁻: 235.1339. found: 235.1360. >95% purity (as determined by RP-HPLC, method C, t_{R}=5.57 min, method G, t_{R}=5.23 min).

### 5-Hydroxy-1-(4-methoxyphenyl)octan-3-one (12f)

Compound 12f was prepared as a colorless oil with Compound 11f (40 mg, 0.161 mmol) as a colorless oil in 80% yield as described in General preparation method B. R_{f}=0.40 (Hexane/EtOAc=1:1, v/v). ¹H NMR (600 MHz, CDCl₃) δ 7.09 (d, J=8.5 Hz, 2H), 6.82 (d, J=8.4 Hz, 2H), 4.03 (brs, 1H), 3.78 (s, 3H), 2.93 (s, 1H), 2.84 (t, J=7.5 Hz, 2H), 2.73 (t, J=7.6 Hz, 2H), 2.57-2.54 (m, 1H), 2.50-2.46 (m, 1H), 1.49-1.29 (m, 4H), 0.91 (t, J=7.1 Hz, 3H); ¹³C NMR (150 MHz, CDCl₃) δ 211.46, 158.03, 132.75, 129.40, 129.22, 113.96, 67.35, 55.27, 49.32, 45.34, 38.57, 28.70, 18.65, 13.98. HRMS m/z calculated for C₁₅H₂₂O₃[M-H]⁻: 249.1496. found: 249.1527. >95% purity (as determined by RP-HPLC, method C, t_{R}=13.20 min, method G, t_{R}=14.78 min).

### 1-(4-Fluorophenyl)-5-hydroxyoctan-3-one (122)

Compound 12g was prepared as a colorless oil with Compound 11g (84 mg, 0.356 mmol) in 65% yield as described in General preparation method B. R_{f}=0.41 (Hexane/EtOAc=1:1, v/v). ¹H NMR (600 MHz, CDCl₃) δ 7.14-7.12 (m, 3H), 6.96 (t, J=8.7 Hz, 2H), 5.56 (s, 1H), 4.04-4.03 (brs, 1H), 4.05 (brs, 1H), 2.90 (s, 1H), 2.87 (t, J=7.6 Hz, 2H), 2.74 (t, J=7.5 Hz, 2H), 2.57-2.54 (m, 1H), 2.51-2.47 (m, 1H), 1.49-1.32 (m, 4H), 0.92 (t, J=7.1 Hz, 3H); ¹³C NMR (150 MHz, CDCl₃) δ 210.91, 162.22, 160.60, 136.41, 136.40, 129.73, 129.68, 115.35, 115.20, 67.36, 49.38, 45.09, 38.63, 28.64, 18.64, 13.96%. HRMS m/z calculated for C₁₄H₁₉FO₂ [M-H]⁻: 237.1296. found: 237.1309. >95% purity (as determined by RP-HPLC, method C, t_{R}=12.21 min, method G, t_{R}=18.56 min).

### 1-(4-Chlorophenyl)-5-hydroxyoctan-3-one (12h)

Compound 12h was prepared as a colorless oil with Compound 11h (48 mg, 0.190 mmol) in 40% yield as described in General preparation method B. R_{f}=0.45 (Hexane/EtOAc=1:1, v/v). ¹H NMR (600 MHz, CDCl₃) δ 7.30-7.26 (m, 2H), 7.21-7.17 (m, 2H), 4.05-4.03 (m, 1H), 2.91 (t, J=7.6 Hz, 2H), 2.76 (t, J=7.6 Hz, 2H), 2.58-2.55 (m, 1H), 2.51-2.47 (m, 1H), 1.51-1.31 (m, 4H), 0.92 (t, J=7.1 Hz, 3H); ¹³C NMR (150 MHz, CDCl₃) δ 211.24, 140.75, 128.56, 128.29, 126.23, 67.36, 49.31, 45.07, 38.59, 29.53, 18.65, 13.98. HRMS m/z calculated for C₁₄H₁₉ClO₂ [M-H]⁻: 253.1001. found: 253.1430. >95% purity (as determined by RP-HPLC, method C, t_{R}=11.95 min, method G, t_{R}=16.89 min).

### 5-Hydroxy-1-(p-tolyl)octan-3-one (12i)

Compound 12i was prepared as a yellow oil with Compound 11i (107 mg, 0.461 mmol) in 70% yield as described in General preparation method B. R_{f}=0.48 (Hexane/EtOAc=1:1, v/v). ¹H NMR (600 MHz, CDCl₃) δ 7.07 (dd, J=8.0 and 18.2 Hz, 4H), 4.05-4.02 (m, 1H), 2.92 (d, J=3.5 Hz, 1H), 2.86 (t, J=7.6 Hz, 2H), 2.74 (t, J=7.6 Hz, 2H), 2.57-2.54 (m, 1H), 2.50-2.46 (m, 1H), 2.31 (s, 3H), 1.49-1.31 (m, 4H), 0.91 (t, J=7.2 Hz, 3H); ¹³C NMR (150 MHz, CDCl₃) δ 211.32, 137.66, 135.69, 129.23, 128.17, 67.36, 49.36, 45.21, 38.65, 29.13, 21.00, 18.67, 14.00. HRMS m/z calculated for C₁₅H₂O₂ [M-H]⁻: 233.1547. found: 233.1575. >95% purity (as determined by RP-HPLC, method C, t_{R}=21.76 min, method G, t_{R}=32.04 min).

### 1-(4-(Dimethylamino)phenyl)-5-hydroxyoctan-3-one (12i)

Compound 12j was prepared as a colorless oil with Compound 11j (80 mg, 0.306 mmol) in 63% yield as described in General preparation method B. R_{f}=0.60 (Hexane/EtOAc=1:1, v/v). ¹H NMR (600 MHz, CDCl₃) δ 7.04 (d, J=8.1 Hz, 2H), 6.68 (d, J=8.1 Hz, 2H), 4.02 (brs, 1H), 2.98 (s, 1H), 2.90 (s, 6H), 2.80 (t, J=7.3 Hz, 2H), 2.70 (t, J=7.5 Hz, 2H), 2.57-2.54 (m, 1H), 2.50-2.45 (m, 1H), 1.47-1.33 (m, 4H), 0.91 (t, J=6.8 Hz, 3H); ¹³C NMR (150 MHz, CDCl₃) δ 211.83, 149.28, 128.88, 128.66, 113.04, 67.35, 49.30, 45.53, 40.83, 38.59, 28.68, 18.67, 14.00. HRMS m/z calculated for C₁₆H₂₅NO₂ [M-H]⁻: 262.1812. found: 262.1828. >95% purity (as determined by RP-HPLC, method D, t_{R}=6.20 min, method G, t_{R}=23.83 min).

### 1-(3,4-Dihydroxyphenyl)-5-hydroxyoctan-3-one (12k)

Compound 12k was prepared as a colorless oil with Compound 11k (36 mg, 0.144 mmol) in 93% yield as described in General preparation method B. R_{f}=0.55 (Hexane/EtOAc=1:3, v/v). ¹H NMR (600 MHz, MeOD) δ 6.67 (d, J=8.2 Hz, 1H), 6.64 (d, J=1.8 Hz, 1H), 6.52 (dd, J=1.8 and 8.2 Hz, 1H), 4.07-3.98 (m, 1H), 2.80-2.69 (m, 4H), 2.57 (q, J=8.2 Hz, 1H), 2.50 (dd, J=4.4 and 15.4 Hz, 1H), 1.53-1.28 (m, 4H), 0.93 (t, J=7.2 Hz, 3H); ¹³C NMR (150 MHz, CDCl₃) δ 210.85, 144.78, 143.07, 132.68, 119.13, 115.09, 114.95, 67.25, 49.93, 45.01, 39.18, 28.65, 18.36, 12.91. HRMS m/z calculated for C₁₆H₂₅NO₂ [M-H]⁻: 251.1289. found: 251.1387. >95% purity (as determined by RP-HPLC, method A, t_{R}=4.86 min, method G, t_{R}=3.93 min).

### 1-(3,4-Dimethoxyphenyl)-5-hydroxyoctan-3-one (12l)

Compound 12l was prepared as a colorless oil with Compound 11l (59 mg, 0.212 mmol) in 63% yield as described in General preparation method B. R_{f}=0.33 (Hexane/EtOAc=1:1, v/v). ¹H NMR (600 MHz, CDCl₃) δ 6.78 (d, J=8.4 Hz, 1H), 6.71-6.70 (m, 2H), 4.04 (brs, 1H), 3.87 (s, 3H), 3.85 (s, 3H), 2.92 (s, 1H), 2.85 (t, J=7.5 Hz, 2H), 2.74 (t, J=7.5 Hz, 2H), 2.58-2.55 (m, 1H), 2.52-2.47 (m, 1H), 1.48-1.32 (m, 4H), 0.92 (t, J=7.1 Hz, 3H); ¹³C NMR (150 MHz, CDCl₃) δ 211.40, 148.93, 147.45, 133.35, 120.07, 111.67, 111.34, 67.38, 55.93, 55.85, 49.37, 45.34, 38.60, 29.20, 18.65, 13.98. HRMS m/z calculated for C₁₆H₂₄O₄ [M-H]⁻: 279.1602. found: 279.1620. >95% purity (as determined by RP-HPLC, method C, t_{R}=8.52 min, method G, t_{R}=8.61 min).

### 1-(3-Fluoro-4-hydroxyphenyl)-5-hydroxyoctan-3-one (12m)

Compound 12m was prepared as a colorless oil with Compound 11m (55 mg, 0.218 mmol) in 63% yield as described in General preparation method B. R_{f}=0.66 (Hexane/EtOAc=1:1, v/v). ¹H NMR (600 MHz, MeOD) δ 6.91 (d, J=12.2 Hz, 1H), 6.82-6.81 (m, 2H), 4.04 (brs, 1H), 2.79 (s, 4H), 2.59-2.55 (m, 1H), 2.53-2.49 (m, 1H), 1.46-1.31 (m, 4H), 0.94 (t, J=7.1 Hz, 3H); ¹³C NMR (150 MHz, MeOD) δ 210.17, 152.10, 150.51, 142.74, 142.66, 133.11, 133.07, 123.85, 123.83, 117.20, 117.19, 115.41, 115.29, 67.24, 49.85, 44.62, 39.21, 28.11, 18.36, 12.91. HRMS m/z calculated for C₁₄H₁₉FO₃ [M-H]⁻: 253.1245. found: 253.1275. >95% purity (as determined by RP-HPLC, method C, t_{R}=6.38 min, method G, t_{R}=6.04 min).

### 1-(3-Ethoxy-4-hydroxyphenyl)-5-hydroxyoctan-3-one (12n)

Compound 12n was prepared as a colorless oil with Compound 1 in (60 mg, 0.216 mmol) in 45% yield as described in General preparation method B. R_{f}=0.35 (Hexane/EtOAc=1:1, v/v). ¹H NMR (600 MHz, CDCl₃) δ 6.82 (d, J=8.0 Hz, 1H), 6.67 (s, 1H), 6.65 (d, J=8.0 Hz, 1H), 5.56 (s, 1H), 4.10-4.07 (q, J=7.0 Hz, 1H), 4.05 (brs, 1H), 2.92 (s, 1H), 2.82 (t, J=7.5 Hz, 2H), 2.72 (t, J=7.5 Hz, 2H), 2.57-2.54 (m, 1H), 2.50-2.46 (m, 1H), 1.48-1.31 (m, 7H), 1.25 (s, 1H), 0.91 (t, J=7.1 Hz, 3H); ¹³C NMR (150 MHz, CDCl₃) δ 211.49, 145.73, 144.12, 132.56, 120.65, 114.34, 111.93, 67.38, 64.44, 49.37, 45.46, 38.59, 29.29, 18.65, 14.91, 13.97. HRMS m/z calculated for C₁₆H₂₄O₄ [M-H]⁻: 279.1602. found: 279.1646. >95% purity (as determined by RP-HPLC, method C, t_{R}=8.06 min, method G, t_{R}=7.73 min).

### 1-(3,4-Difluorophenyl)-5-hydroxyoctan-3-one (12o)

Compound 12o was prepared as a colorless oil with Compound 11o (49 mg, 0.193 mmol) in 81% yield as described in General preparation method B. R_{f}=0.40 (Hexane/EtOAc=1:1, v/v). ¹H NMR (600 MHz, CDCl₃) δ 7.05 (q, J=9.0 Hz, 1H), 6.98 (t, J=9.5 Hz, 1H), 6.89-6.87 (m, 1H), 4.07-4.04 (m, 1H), 2.86 (t, J=7.4 Hz, 2H), 2.80 (s, 1H), 2.74 (t, J=7.4 Hz, 2H), 2.58-2.54 (m, 1H), 2.52-2.48 (m, 1H), 1.49-1.33 (m, 4H), 0.92 (t, J=7.1 Hz, 3H); ¹³C NMR (150 MHz, CDCl₃) δ 210.35, 151.03, 150.95, 149.77, 149.69, 149.39, 149.31, 148.14, 148.05, 137.78, 137.75, 137.74, 137.72, 124.21, 124.18, 124.16, 124.14, 117.21, 117.19, 117.10, 117.08, 67.38, 49.38, 44.66, 38.66, 28.51, 18.64, 13.94. HRMS m/z calculated for C₁₄H₁₈F₂O₂ [M-H]⁻: 255.1202. found: 255.1235. >95% purity (as determined by RP-HPLC, method C, t_{R}=18.49 min, method G, t_{R}=21.95 min).

### 1-(3,4-Dimethylphenyl)-5-hydroxyoctan-3-one (12u)

Compound 12p was prepared as a colorless oil with Compound 11p (83 mg, 0.337 mmol) in 72% yield as described in General preparation method B. R_{f}=0.60 (Hexane/EtOAc=1:1, v/v). ¹H NMR (600 MHz, CDCl₃) δ 7.04 (d, J=7.6 Hz, 1H), 6.94 (s, 1H), 6.90 (d, J=7.6 Hz, 1H), 4.05-4.01 (m, 1H), 2.94 (d, J=3.5 Hz, 1H), 2.83 (t, J=7.6 Hz, 2H), 2.73 (t, J=7.6 Hz, 2H), 2.58-2.55 (m, 1H), 2.51-2.46 (m, 1H), 2.23 (s, 3H), 2.22 (s, 3H), 1.49-1.31 (m, 4H), 0.91 (t, J=7.2 Hz, 3H); ¹³C NMR (150 MHz, CDCl₃) δ 211.43, 138.14, 136.67, 134.35, 129.80, 129.64, 125.57, 67.37, 49.32, 45.29, 38.63, 29.12, 19.75, 19.31, 18.67, 14.00. HRMS m/z calculated for C₁₆H₂₄O₂ [M-H]⁻: 247.1703. found: 247.1725. >95% purity (as determined by RP-HPLC, method C, t_{R}=31.01 min, method G, t_{R}=21.80 min).

### 1-(4-Fluoro-3-methoxyphenyl)-5-hydroxyoctan-3-one (12a)

Compound 12q was prepared as a colorless oil with Compound 11q (48 mg, 0.180 mmol) in 62% yield as described in General preparation method B. R_{f}=0.50 (Hexane/EtOAc=1:1, v/v). ¹H NMR (600 MHz, CDCl₃) δ 6.97 (dd, J=8.2 Hz, 11.3 Hz, 1H), 6.78 (dd, J=8.1 and 1.9 Hz, 1H), 6.69-6.67 (m, 1H), 4.06-4.03 (m, 1H), 3.87 (s, 3H), 2.88-2.84 (m, 3H), 2.75 (t, J=7.4 Hz, 2H), 2.58-2.55 (m, 1H), 2.52-2.48 (m, 1H), 1.50-1.31 (m, 4H), 0.92 (t, J=7.1 Hz, 3H); ¹³C NMR (150 MHz, CDCl₃) δ 210.91, 151.86, 150.24, 147.46, 147.39, 137.10, 137.08, 120.30, 120.25, 115.95, 115.83, 113.67, 67.38, 56.23, 49.40, 45.10, 38.63, 29.15, 18.64, 13.96. HRMS m/z calculated for C₁₅H₂₁FO₃ [M-H]⁻: 267.1402. found: 267.1426. >95% purity (as determined by RP-HPLC, method C, t_{R}=14.58 min, method G, t_{R}=14.25 min).

### 1-(3-Fluoro-5-methoxyphenyl)-5-hydroxyoctan-3-one (12r)

Compound 12r was prepared as a colorless oil with Compound 11r (43 mg, 0.162 mmol) in 40% yield as described in General preparation method B. R_{f}=0.45 (Hexane/EtOAc=1:1, v/v). ¹H NMR (600 MHz, CDCl₃) δ 6.51 (s, 1H), 6.49-6.44 (m, 2H), 4.06-4.04 (m, 1H), 3.77 (s, 3H), 2.86-2.84 (m, 3H), 2.75 (t, J=7.6 Hz, 2H), 2.59-2.55 (m, 1H), 2.52-2.48 (m, 1H), 1.50-1.32 (m, 4H), 0.92 (t, J=7.1 Hz, 3H); ¹³C NMR (150 MHz, CDCl₃) δ 210.64, 164.48, 162.85, 160.97, 160.90, 143.85, 143.79, 110.03, 110.01, 107.45, 107.31, 99.37, 99.20, 67.37, 55.47, 49.34, 44.53, 38.62, 29.38, 29.36, 18.65, 13.96. HRMS m/z calculated for C₁₅H₂₁FO₃ [M-H]⁻: 267.1402. found: 267.1419. >95% purity (as determined by RP-HPLC, method C, t_{R}=17.95 min, method G, t_{R}=22.68 min).

### (E)-1-(3,4-Difluorophenyl)oct-1-en-3-one (13a) (Formula 13a)

A 3,4-difluorobenzaldehyde (500 mg, 3.52 mmol) solution in MeOH (8 mL) as a solvent was added to (L)-proline (61.0 mg, 0.53 mmol) and heptan-2-one (0.49 mL, 3.52 mmol) under argon at 25° C. After 30 minutes, trimethylamine (0.12 mL, 0.88 mmol) was added to the solution, stirred for 72 hours at 25° C., quenched with water, and extracted with EtOAc (3×25 mL). The mixed organic layer was dried with MgSO₄ and filtered, followed by concentration under reduced pressure. The residue was purified by silica gel column chromatography (Hexane/EtOAc=12:1 to 6:1, v/v), thereby obtaining Compound 13a as a colorless oil in 50% yield. R_{f}=0.75 (Hexane/EtOAc=4:1, v/v). ¹H NMR (600 MHz, CDCl₃) δ 7.45 (d, J=16.1 Hz, 1H), 7.40-7.35 (m, 1H), 7.30-7.26 (m, 1H), 7.21-7.15 (m, 1H), 6.65 (d, J=16.1 Hz, 1H), 2.64 (t, J=7.4 Hz, 2H), 1.72-1.63 (m, 2H), 1.40-1.29 (m, 4H), 0.91 (t, J=7.2 Hz, 3H); ¹³C NMR (150 MHz, CDCl₃) δ 200.05, 152.38, 152.29, 151.48, 151.40, 150.69, 150.61, 149.83, 149.74, 139.76, 131.93, 131.90, 131.86, 126.95, 126.93, 125.05, 125.02, 125.00, 124.98, 117.90, 117.78, 116.41, 116.30, 41.27, 31.46, 23.91, 22.48, 13.92. HRMS m/z calculated for C₁₄H₁₆F₂O [M-H]⁻: 237.1096. found: 237.1126. >95% purity (as determined by RP-HPLC, method E, t_{R}=16.72 min, method I, t_{R}=11.93 min).

### (E)-1-(4-Hydroxy-3-methoxyphenyl)oct-1-en-3-one (13b) (Formula 13b)

Compound 13b was prepared as a yellow oil in 30% yield in the same manner as described in the method of synthesizing Compound 13a, except that vanillin (500 mg, 3.29 mmol), instead of 3,4-difluorobenaldehyde, was used. R_{f}=0.44 (Hexane/EtOAc=5:1, v/v). ¹H NMR (600 MHz, CDCl₃) δ 7.48 (d, J=16.0 Hz, 1H), 7.10 (dd, J=1.6 and 8.2 Hz, 1H), 7.05 (d, J=1.7 Hz, 1H), 6.93 (d, J=8.2 Hz, 1H), 6.60 (d, J=15.6 Hz, 1H), 6.00 (brs, 1H), 3.93 (s, 3H), 2.64 (t, J=7.2 Hz, 2H), 1.81-1.69 (m, 2H), 1.49-1.21 (m, 4H), 0.87 (t, J=7.2 Hz, 3H); ¹³C NMR (150 MHz, CDCl₃) δ 200.93, 148.23, 146.94, 142.74, 127.05, 124.06, 123.38, 114.88, 109.52, 55.95, 40.64, 31.56, 24.26, 22.52, 13.97. HRMS m/z calculated for C₁₅H₂₀O₃ [M-H]⁻: 247.1339. found: 247.1406. >95% purity (as determined by RP-HPLC, method C, t_{R}=18.10 min, method H, t_{R}=13.32 min).

### 1-(3,4-Difluorophenyl)octan-3-one (14a) (Formula 14a)

Compound 14a was prepared as a colorless oil with Compound 13a (30 mg, 0.126 mmol) in 76% yield as described in General preparation method B. R_{f}=0.77 (Hexane/EtOAc=4:1, v/v). ¹H NMR (600 MHz, CDCl₃) δ 7.10-7.01 (m, 1H), 7.01-6.94 (m, 1H), 6.92-6.82 (m, 1H), 2.85 (t, J=7.4 Hz, 2H), 2.70 (t, J=7.4 Hz, 2H), 2.37 (t, J=7.4 Hz, 2H), 1.59-1.51 (m, 2H), 1.34-1.19 (m, 4H), 0.88 (t, J=7.2 Hz, 3H); ¹³C NMR (150 MHz, CDCl₃) δ 209.73, 151.00, 150.92, 149.70, 149.62, 149.36, 149.28, 148.07, 147.99, 138.20, 138.17, 138.14, 124.24, 124.22, 124.21, 124.19, 117.21, 117.12, 117.10, 117.01, 43.80, 43.05, 31.36, 28.80, 23.47, 22.43, 13.89. HRMS m/z calculated for C₁₄H₁₈F₂O [M-H]⁻: 239.1253. found: 239.1288. >95% purity (as determined by RP-HPLC, method H, t_{R}=10.08 min, method I, t_{R}=11.68 min).

### 1-(4-Hydroxy-3-methoxyphenyl)octan-3-one (14b) (Formula 14b)

Compound 14b was prepared as a colorless oil with Compound 13b (21 mg, 0.085 mmol) in 75% yield as described in General preparation method B. R_{f}=0.51 (Hexane/EtOAc=5:1, v/v). ¹H NMR (600 MHz, CDCl₃) δ 6.81 (d, J=7.8 Hz, 1H), 6.69 (s, 1H), 6.67 (d, J=7.8 Hz, 1H), 5.47 (brs, 1H), 3.87 (s, 3H), 2.82 (t, J=7.8 Hz, 2H), 2.69 (t, J=7.8 Hz, 2H), 2.37 (t, J=7.8 Hz, 2H), 1.68-1.51 (m, 2H), 1.38-1.21 (m, 6H), 0.87 (t, J=7.2 Hz, 3H); ¹³C NMR (150 MHz, CDCl₃) δ 210.65, 146.38, 143.87, 133.15, 120.78, 114.31, 111.06, 55.88, 44.63, 43.12, 31.40, 30.33, 29.71, 29.56, 23.50, 22.46, 13.92. HRMS m/z calculated for C₁₅H22O₃ [M-H]⁻: 249.1496. found: 249.1531. >95% purity (as determined by RP-HPLC, method C, t_{R}=17.50 min, method H, t_{R}=9.64 min).

### (E)-1-(3,4-Difluorophenyl)oct-1-en-3-ol (15a) (Formula 15a)

A stirred Compound 13a (55.0 mg, 0.231 mmol) solution in MeOH (8 mL) as a solvent was added to NaBH₄ (12.2 mg, 0.315 mmol) at 0° C. The reaction mixture was stirred at room temperature for 1 hour, concentrated, and extracted with EtOAc (3×25 mL). The organic layer was washed with water, dried with MgSO₄, filtered and concentrated under reduced pressure. The residue was purified by silica gel column chromatography (Hexane/EtOAc=8:1, v/v), thereby obtaining Compound 15a as a colorless oil in 94% yield. R_{f}=0.51 (Hexane/EtOAc=4:1, v/v). ¹H NMR (600 MHz, CDCl₃) δ 7.23-7.16 (m, 1H), 7.14-7.06 (m, 2H), 6.50 (d, J=15.9 Hz, 1H), 6.16 (dd, J=6.5 and 13.1 Hz, 1H), 4.29 (q, J=6.5 Hz, 1H), 1.78 (brs, 1H), 1.71-1.55 (m, 2H), 1.51-1.25 (m, 6H), 0.92 (t, J=7.2 Hz, 3H); ¹³C NMR (150 MHz, CDCl₃) δ 151.25, 150.65, 150.57, 149.70, 149.61, 149.00, 148.92, 134.12, 134.08, 134.05, 133.76, 133.75, 128.02, 122.66, 122.64, 122.62, 122.60, 117.34, 117.23, 114.79, 114.68, 72.73, 37.35, 31.76, 25.10, 22.60, 14.03. HRMS m/z calculated for C₁₄H₁₈F₂O [M-H]⁻: 239.1253. found: 239.1290. >95% purity (as determined by RP-HPLC, method E, t_{R}=10.05 min, method I, t_{R}=9.67 min).

### (E)-4-(3-Hydroxyoct-1-en-1-yl)-2-methoxyphenol (15b) (Formula 15b)

Compound 15b was prepared as a brown oil in 90% yield in the same manner as described in the method of synthesizing Compound 15a, except that compound 15a, instead of Compound 13a, was used. R_{f}=0.36 (Hexane/EtOAc=2:1, v/v). ¹H NMR (600 MHz, CDCl₃) δ 6.94 (d, J=1.6 Hz, 1H), 6.92-6.88 (m, 2H), 6.50 (d, J=15.8 Hz, 1H), 6.08 (q, J=7.1 Hz, 1H), 5.67 (s, 1H), 4.27 (dd, J=6.5 and 13.3 Hz, 1H), 3.93 (s, 3H), 1.72-1.58 (m, 4H), 1.37-1.30 (m, 4H), 0.92 (t, J=7.2 Hz, 3H); ¹³C NMR (150 MHz, CDCl₃) δ 146.63, 145.52, 130.33, 130.31, 129.33, 120.29, 114.4, 108.27, 73.33, 55.90, 37.42, 31.81, 25.21, 22.63, 14.07. HRMS m/z calculated for C₁₅H₂₂O₃ [M-H]⁻: 249.1496. found: 249.1525. >95% purity (as determined by RP-HPLC, method C, t_{R}=11.86 min, method H, t_{R}=9.02 min).

### 1-(3,4-Difluorophenyl)octan-3-ol (16a) (Formula 16a)

Compound 16a was prepared as a colorless oil with Compound 15a (30 mg, 0.125 mmol) in 60% yield as described in General preparation method B. R_{f}=0.58 (Hexane/EtOAc=4:1, v/v). ¹H NMR (600 MHz, CDCl₃) δ 7.09-7.02 (m, 1H), 7.02-6.96 (m, 1H), 6.93-6.86 (m, 1H), 3.64-3.55 (m, 1H), 2.81-2.72 (m, 1H), 2.67-2.59 (m, 1H), 1.80-1.64 (m, 2H), 1.53-1.23 (m, 9H), 0.89 (t, J=7.2 Hz, 3H); ¹³C NMR (150 MHz, CDCl₃) δ 151.02, 150.94, 149.54, 149.45, 149.38, 149.30, 147.91, 147.83, 139.23, 139.20, 139.17, 124.18, 124.16, 124.14, 124.12, 117.14, 117.03, 117.01, 116.90, 71.09, 38.80, 37.68, 31.84, 31.24, 25.28, 22.62, 14.02. HRMS m/z calculated for C₁₄H₂₀F₂O [M-H]⁻: 241.1409. found: 241.1452. >95% purity (as determined by RP-HPLC, method E, t_{R}=12.84 min, method I, t_{R}=14.86 min).

### 4-((Tert-butyldimethylsilyl)oxy)-3-methoxybenzaldehyde (17)

Vanillin (1.00 g, 6.57 mmol) was dissolved in anhydrous CH₂Cl₂ (50 mL) and stirred, followed by adding imidazole (1.29 g, 18.96 mmol) and tert-butyldimethylsilyl chloride (1.42 g, 9.39 mmol) at 0° C. The reaction mixture was stirred under argon at room temperature for 16 hours, quenched with water, and then extracted with CH₂Cl₂ (3×25 mL). The organic layer was washed with water, dried with MgSO₄, filtered and concentrated under reduced pressure. The residue was purified by silica gel column chromatography (Hexane/EtOAc=4:1, v/v), thereby obtaining Compound 17 as a colorless oil in 94% yield. R_{f}=0.89 (Hexane/EtOAc=1:1, v/v). ¹H NMR (600 MHz, CDCl₃) δ 9.85 (s, 1H), 7.40 (d, J=1.9 Hz, 1H), 7.37 (dd, J=4.0 and 8.0 Hz, 1H), 6.97 (d, J=8.0 Hz, 1H), 3.87 (s, 3H), 1.00 (s, 9H), 0.20 (s, 6H); ¹³C NMR (150 MHz, CDCl₃) δ 191.19, 151.80, 151.51, 131.09, 126.40, 120.87, 110.25, 55.61, 25.74, 18.66, −4.41.

### Tert-butyl(4-(2,2-dibromovinyl)-2-methoxyphenoxy)dimethylsilane (18)

CBr₄ (3.37 g, 10.14 mmol) was dissolved in anhydrous CH₂Cl₂ (30 mL) and stirred, and slowly added to PPh₃ (5.32 g, 20.28 mmol) at 0° C. The resulting mixture was stirred under argon at the same temperature for 1 hour, and then Compound 17 (1.35 g, 5.07 mmol) in anhydrous CH₂Cl₂ (10 mL) was slowly added for 10 minutes or more. The reaction mixture was stirred under argon for 2 hours, and extracted with CH₂Cl₂ (3×25 mL). The organic layer was washed with water, dried with MgSO₄, and concentrated under reduced pressure. The residue was purified by silica gel column chromatography (Hexane/EtOAc=15:1, v/v), thereby obtaining Compound 18 as a colorless oil in 98% yield. R_{f}=0.87 (Hexane/EtOAc=8:1, v/v). ¹H NMR (600 MHz, CDCl₃) δ 7.42 (s, 1H), 7.19 (d, J=2.0 Hz, 1H), 7.03 (dd, J=4.1 and 8.3 Hz, 1H), 6.84 (d, J=8.2 Hz, 1H), 3.83 (s, 3H), 1.02 (s, 9H), 0.19 (s, 6H); ¹³C NMR (150 MHz, CDCl₃) δ 150.62, 145.64, 136.59, 128.77, 121.96, 120.68, 112.03, 87.10, 55.51, 25.69, 25.66, 18.47, −4.58.

### 4-(2,2-Dibromovinyl)-1,2-difluorobenzene (28)

Compound 28 was prepared as a colorless oil in 97% yield in the same manner as described in the method of synthesizing Compound 18, except that 3,4-difluorobenzaldehyde (1.35 g, 9.50 mmol), instead of Compound 17, was used. The residue was purified by silica gel column chromatography (Hexane/EtOAc=10:1, v/v). R_{f}=0.85 (Hexane/EtOAc=4:1, v/v). ¹H NMR (600 MHz, CDCl₃) δ 7.46 (q, J=9.6 Hz, 1H), 7.39 (s, 1H), 7.23-7.20 (m, 1H), 7.15 (q, J=9.1 Hz, 1H); ¹³C NMR (150 MHz, CDCl₃) δ 151.01, 150.94, 150.86, 149.35, 149.29, 149.27, 149.21, 134.73, 132.19, 132.15, 125.18, 125.15, 125.13, 125.11, 117.42, 117.30, 117.26, 117.14, 116.60, 91.07, 91.06.

### tert-Butyl(4-ethynyl-2-methoxyphenoxy)dimethylsilane (19)

A stirred Compound 18 (1.41 g, 3.34 mmol) in anhydrous THF (20 mL) as a solvent was added to n-BuLi (1.6 M in hexanes, 5.30 mL, 8.35 mmol) at −78° C. The reaction mixture was stirred under argon at the same temperature for 2 hours, quenched with a NH₄Cl aqueous solution (10 mL), and extracted with EtOAc (3×25 mL). The organic layer was washed with water, dried with MgSO₄, and concentrated under reduced pressure. The residue was purified by silica gel column chromatography (Hexane/EtOAc=16:1 to 10:1, v/v), thereby obtaining Compound 19 as a colorless oil in 96% yield. R_{f}=0.80 (Hexane/EtOAc=8:1, v/v). ¹H NMR (600 MHz, CDCl₃) δ 7.00 (dd, J=4.0 and 8.1 Hz, 1H), 6.97 (d, J=1.9 Hz, 1H), 3.80 (s, 3H), 2.99 (s, 1H), 0.99 (s, 9H), 0.15 (s, 6H); ¹³C NMR (150 MHz, CDCl₃) δ 150.66, 146.23, 125.46, 120.90, 115.66, 115.04, 83.96, 75.57, 55.45, 25.67, 18.47, −4.64.

### 4-Ethynyl-1,2-difluorobenzene (29)

A stirred Compound 28 (800 mg, 2.69 mmol) solution in anhydrous THF (40 mL) as a solvent was added to n-BuLi (1.6 M in hexanes, 4.30 mL, 6.71 mmol) at −78° C. The reaction mixture was stirred under argon at the same temperature for 2 hours, quenched with an NH₄Cl aqueous solution (10 mL), and extracted with hexane (3×25 mL). The organic layer was washed with water, dried with MgSO₄, filtered and concentrated under reduced pressure at a low temperature (20° C.), thereby preparing Compound 29 to be used in subsequent steps without additional purification.

### General Preparation Method C for Compounds 20 and 30 to 32

An appropriate ethynylbenzene (400 mg) solution stirred in a THF (40 mL) solvent was added to n-BuLi (1.1 eq.) at −78° C. The solution was stirred under argon (Ar) at the same temperature for 1 hour, and appropriate Weinreb amides (1.5 eq.) were slowly added dropwise. The reaction mixture was stirred (generally, for 10 to 12 hours) in the presence of argon at the same temperature until a TLC analysis result showed a complete conversion of a starting material to a product, quenched with an aqueous NH₄Cl solution (10 mL), and extracted with EtOAc (3×25 mL). An organic layer was washed with brine, dried with MgSO₄, filtered and concentrated under reduced pressure. The residue was purified by silica gel column chromatography (hexane/ether=120:1 to 80:1, v/v), thereby obtaining Compounds 20 and 30 to 32.

### 1-(4-((tert-Butyldimethylsilyl)oxy)-3-methoxyphenyl)oct-1-yn-3-one (20)

Compound 20 was prepared as a colorless oil with Compound 19 (400 mg, 1.52 mmol) and N-methoxy-N-methylhexanamide (364 mg, 2.28 mmol) in 75% yield as described above in General Preparation Method C. R_{f}=0.75 (Hexane/EtOAc=8:1, v/v). ¹H NMR (600 MHz, CDCl₃) δ 7.12 (d, J=8.1 Hz, 1H), 7.07 (s, 1H), 6.84 (d, J=8.2 Hz, 1H), 3.82 (s, 3H), 2.65 (t, J=7.4 Hz, 2H), 1.77-1.75 (m, 2H), 1.38-1.37 (m, 4H), 1.00 (s, 9H), 0.93 (t, J=6.5 Hz, 3H), 0.18 (s, 6H); ¹³C NMR (150 MHz, CDCl₃) δ 188.36, 150.90, 148.19, 127.18, 121.14, 116.48, 112.74, 91.88, 87.48, 55.51, 45.41, 31.21, 25.62, 23.99, 22.43, 18.48, 13.92, −4.59.

### 1-(3,4-Difluorophenyl)hex-1-yn-3-one (30) (Formula 30)

Compound 30 was prepared as a colorless oil with Compound 29 (400 mg, 2.90 mmol) and N-methoxy-N-methylhexanamide (577 mg, 4.40 mmol) in 72% yield as described above in General Preparation Method C. R_{f}=0.69 (Hexane/Ether=6:1, v/v). ¹H NMR (600 MHz, CDCl₃) δ 7.39 (t, J=7.9 Hz, 1H), 7.36-7.32 (m, 1H), 7.19 (q, J=8.4 Hz, 1H), 2.64 (t, J=7.4 Hz, 2H), 1.82-1.72 (m, 2H), 1.00 (t, J=7.2 Hz, 3H); ¹³C NMR (150 MHz, CDCl₃) δ 187.63, 152.92, 152.84, 151.23, 151.14, 150.93, 150.84, 149.26, 149.18, 130.06, 130.04, 130.02, 130.00, 121.97, 121.85, 118.06, 117.94, 116.94, 116.91, 116.89, 116.86, 87.83, 87.53, 47.29, 17.55, 13.48. HRMS m/z calculated for C₁₂H₁₀F₂O [M-H]⁻: 207.0627. found: 207.0630. >95% purity (as determined by RP-HPLC, method E, t_{R}=8.91 min, method I, t_{R}=5.56 min).

### 1-(3,4-Difluorophenyl)hept-1-yn-3-one (31) (Formula 31)

Compound 31 was prepared as a colorless oil with Compound 29 (400 mg, 2.90 mmol) and N-methoxy-N-methylhexanamide (577 mg, 4.40 mmol) in 88% yield as described above in General Preparation Method C. R_{f}=0.72 (Hexane/Ether=6:1, v/v). ¹H NMR (600 MHz, CDCl₃) δ 7.39 (t, J=7.9 Hz, 1H), 7.36-7.29 (m, 1H), 7.19 (q, J=8.4 Hz, 1H), 2.66 (t, J=7.4 Hz, 2H), 1.76-1.68 (m, 2H), 1.44-1.35 (m, 2H), 0.95 (t, J=7.2 Hz, 3H); ¹³C NMR (150 MHz, CDCl₃) δ 187.82, 152.96, 152.88, 151.27, 151.19, 150.97, 150.88, 149.31, 149.22, 130.07, 130.05, 130.03, 130.00, 122.03, 121.90, 118.09, 117.97, 116.88, 87.89, 87.60, 45.21, 26.12, 22.14, 13.87. HRMS m/z calculated for C₁₃H₁₂F₂O [M-H]⁻: 221.0783. found: 221.0813. >95% purity (as determined by RP-HPLC, method E, t_{R}=12.70 min, method I, t_{R}=7.93 min).

### 1-(3,4-Difluorophenyl)oct-1-yn-3-one (32) (Formula 32)

Compound 32 was prepared as a colorless oil with Compound 29 (400 mg, 2.90 mmol) and N-methoxy-N-methylhexanamide (577 mg, 4.40 mmol) in 72% yield as described above in General Preparation Method C. R_{f}=0.79 (Hexane/EtOAc=6:1, v/v). ¹H NMR (600 MHz, CDCl₃) δ 7.39 (t, J=7.9 Hz, 1H), 7.36-7.29 (m, 1H), 7.19 (q, J=8.4 Hz, 1H), 2.65 (t, J=7.4 Hz, 2H), 1.77-1.69 (m, 2H), 1.41-1.30 (m, 4H), 0.92 (t, J=7.2 Hz, 3H); ¹³C NMR (150 MHz, CDCl₃) δ 187.87, 152.89, 151.28, 150.89, 149.23, 130.07, 130.04, 130.02, 130.00, 122.03, 121.91, 118.10, 117.97, 116.96, 87.89, 87.62, 45.46, 31.13, 23.74, 22.39, 13.89. HRMS m/z calculated for C₁₄H₁₄F₂O [M-H]⁻: 235.0940. found: 235.0971. >95% purity (as determined by RP-HPLC, method E, t_{R}=18.50 min, method I, t_{R}=12.86 min).

### General Preparation Method D for Compounds 21, 23a, 23b, 23c, 25, and 27

A stirred compound protected with a silyl group was dissolved in an anhydrous THF solution and added to a tetrabutylammonium fluoride solution (1M in THF, 2.0 eq.) at 0° C. The reaction mixture was stirred (generally, for 1 hour) at the same temperature in the presence of argon until a TLC analysis result showed a complete conversion of a starting material to a product, quenched with an aqueous NH₄Cl solution (10 mL), and extracted with EtOAc (3×25 mL). An organic layer was washed with brine, dried with MgSO₄, filtered and concentrated under reduced pressure. The residue was purified by silica gel column chromatography (hexane/EtOAc), thereby obtaining Compounds 21, 23a, 23b, 23c, 25, and 27.

### 1-(4-Hydroxy-3-methoxyphenyl)oct-1-yn-3-one (21) (Formula 21)

Compound 21 was prepared as a white oil with Compound 20 in 94% yield as described above in General Preparation Method D. R_{f}=0.21 (Hexane/EtOAc=5:1, v/v). ¹H NMR (600 MHz, CDCl₃) δ 7.17 (dd, J=1.8 and 7.8 Hz, 1H), 7.06 (d, J=1.8 Hz, 1H), 6.91 (d, J=7.8 Hz), 5.99 (brs, 1H), 3.91 (s, 3H), 2.64 (t, J=7.8 Hz, 2H), 1.77-1.71 (m, 2H), 1.42-1.33 (m, 4H), 0.92 (t, J=7.1 Hz, 3H); ¹³C NMR (150 MHz, CDCl₃) δ 188.40, 148.53, 146.35, 128.02, 115.18, 114.87, 111.33, 91.93, 87.29, 56.10, 45.37, 31.20, 23.98, 22.43, 13.92. HRMS m/z calculated for C₁₅H₁₈O₃ [M-H]⁻: 245.1183. found: 245.1261. >95% purity (as determined by RP-HPLC, method F, t_{R}=5.67 min, method H, t_{R}=19.33 min).

### 4-(3-Hydroxyoct-1-yn-1-yl)-2-methoxyphenol (23a) (Formula 23)

Compound 23a was prepared as a white oil with Compound 22a in 94% yield as described above in General Preparation Method D. R_{f}=0.21 (Hexane/EtOAc=5:1, v/v). ¹H NMR (600 MHz, CDCl₃) δ 6.98 (dd, J=1.8 and 15.6 Hz, 1H), 6.92 (d, J=1.8 Hz, 1H), 6.84 (d, J=8.4 Hz, 1H), 5.77 (brs, 1H), 4.58 (t, J=6.6 Hz, 1H), 3.88 (s, 3H), 1.92 (brs, 1H), 1.89-1.79 (m, 2H), 1.45-1.32 (m, 4H), 0.91 (t, J=7.2 Hz, 3H); ¹³C NMR (150 MHz, CDCl₃) δ 146.28, 146.13, 125.66, 114.49, 114.23, 113.95, 88.33, 84.97, 63.09, 55.98, 37.97, 31.50, 24.95, 22.59, 14.03. HRMS m/z calculated for C₁₅H₂O₃ [M-H]⁻: 247.1339. found: 247.1397. >95% purity (as determined by RP-HPLC, method C, t_{R}=12.46 min, method H, t_{R}=10.92 min).

### (R)-4-(3-Hydroxyoct-1-yn-1-yl)-2-methoxyphenol (23b) (Formula 23)

Compound 23b was prepared as a white oil with Compound 22b in 94% yield as described above in General Preparation Method D. R_{f}=0.21 (Hexane/EtOAc=5:1, v/v). ¹H NMR (600 MHz, CDCl₃) δ 6.98 (dd, J=1.7 and 8.2 Hz, 1H), 6.92 (d, J=1.7 Hz, 1H), 6.84 (d, J=8.2 Hz, 1H), 5.72 (brs, 1H), 4.58 (q, J=6.4 Hz, 1H), 3.89 (s, 3H), 1.87-1.73 (m, 3H), 1.58-1.47 (m, 2H), 1.39-1.30 (m, 4H), 0.91 (t, J=7.2 Hz, 3H); ¹³C NMR (150 MHz, CDCl₃) δ 146.29, 146.12, 125.67, 114.46, 114.23, 113.93, 88.32, 84.97, 63.10, 55.98, 37.98, 31.50, 24.94, 22.59, 14.03. HRMS m/z calculated for C₁₅H₂₀O₃[M-H]⁻: 247.1339. found: 247.1397. >95% purity (as determined by RP-HPLC, method C, t_{R}=7.43 min, method H, t_{R}=10.95 min).

### (S)-4-(3-Hydroxyoct-1-yn-1-yl)-2-methoxyphenol (23c) (Formula 23)

Compound 23c was prepared as a yellow oil with Compound 22c in 93% yield as described above in General Preparation Method D. R_{f}=0.21 (Hexane/EtOAc=5:1, v/v). ¹H NMR (600 MHz, CDCl₃) δ 6.97 (t, J=6.6 Hz, 1H), 6.92 (d, J=1.8 Hz, 1H), 6.84 (d, J=7.8 Hz, 1H), 4.58 (t, J=6.6 Hz, 1H), 3.88 (s, 3H), 1.89-1.76 (m, 2H), 1.63-1.55 (m, 2H), 1.43-1.31 (m, 4H), 0.90 (t, J=7.2 Hz, 3H); ¹³C NMR (150 MHz, CDCl₃) δ 146.30, 146.15, 125.65, 114.50, 114.21, 113.96, 88.34, 84.97, 63.09, 55.97, 37.97, 31.50, 24.95, 22.59, 14.03. HRMS m/z calculated for C₁₅H₂₀O₃ [M-H]⁻: 247.1339. found: 247.1415. >95% purity (as determined by RP-HPLC, method C, t_{R}=7.70 min, method H, t_{R}=10.95 min).

### 1-(4-((tert-Butyldimethylsilyloxy)-3-methoxyphenyl)oct-1-yn-3-ol (22a)

A stirred Compound 20 (210 mg, 0.582 mmol) solution in 15 mL MeOH as a solvent was added to NaBH₄ (33.0 mg, 0.874 mmol) at 0° C. The reaction mixture was stirred for 1 hour at room temperature in argon, concentrated and extracted with EtOAc (3×25 mL). An organic layer was washed with brine, dried with MgSO₄, filtered and concentrated under reduced pressure. The residue was purified by silica gel column chromatography (hexane/EtOAc=8:1, v/v), thereby obtaining Compound 22a as a colorless oil in 77% yield. R_{f}=0.59 (Hexane/EtOAc=4:1, v/v). ¹H NMR (600 MHz, CDCl₃) δ 6.94-6.92 (m, 2H), 6.77 (d, J=7.9 Hz, 1H), 4.58 (q, J=6.4 Hz, 1H), 3.79 (s, 3H), 1.92 (d, J=5.2 Hz, 1H), 1.81-1.76 (m, 2H), 1.53-1.49 (m, 2H), 1.35-1.34 (m, 4H), 0.98 (s, 9H), 0.91 (t, J=7.0 Hz, 3H), 0.15 (s, 6H); ¹³C NMR (150 MHz, CDCl₃) δ 150.66, 145.84, 124.96, 120.90, 115.69, 115.31, 88.72, 85.01, 63.08, 55.44, 37.97, 31.51, 25.68, 24.95, 22.60, 18.47, 14.04, −4.65.

### (Z)-4-(3-Hydroxyoct-1-en-1-yl)-2-methoxyphenol (25) (Formula 25)

A Compound 22a (70.0 mg, 0.19 mmol) solution in MeOH (3 mL) as a solvent was added to quinoline (7 mg, 0.06 mmol) and a Lindlar catalyst (7 mg, 0.02 mmol), and the reaction mixture was stirred for 1 hour at 0° C. in the presence of hydrogen (balloon). After the complete conversion of Compound 22a (TLC, toluene/EtOAc=10:1, v/v), the reaction mixture was filtered through a Celite pad, and washed with MeOH (3×5 mL). The residue obtained by concentrating the filtrate under reduced pressure was purified by silica gel column chromatography (toluene/EtOAc=10:1, v/v), thereby obtaining Compound 24 as a colorless oil. R_{f}=0.36 (toluene/EtOAc=10:1, v/v). ¹H NMR (600 MHz, CDCl₃) δ 6.83 (d, J=1.8 Hz, 1H), 6.81 (d, J=8.4 Hz, 1H), 6.77 (dd, J=1.8 and 8.1 Hz, 1H), 6.46 (d, J=11.4 Hz, 1H), 5.57 (dd, J=9.0 and 12.0 Hz, 1H), 4.60 (dd, J=7.2 and 15.3 Hz, 1H), 3.80 (s, 3H), 1.69-1.62 (m, 1H), 1.61 (brs, 1H), 1.59-1.52 (m, 1H), 1.46-1.21 (m, 6H), 1.00 (s, 9H), 0.88 (t, J=6.6 Hz, 3H), 0.16 (s, 6H).

Compound 25 was prepared as a colorless oil with Compound 24 in 70% yield as described above in General Preparation Method D. R_{f}=0.21 (toluene/EtOAc=10:1, v/v). ¹H NMR (600 MHz, CDCl₃) δ 6.89 (d, J=8.4 Hz, 1H), 6.88-6.86 (m, 1H), 6.85-6.81 (m, 1H), 6.48 (d, J=11.4 Hz, 1H), 5.64 (s, 1H), 5.57 (dd, J=9.0 and 11.7 Hz, 1H), 4.56-4.51 (m, 1H), 3.90 (s, 3H), 1.71-1.63 (m, 1H), 1.61-1.54 (m, 1H), 1.52 (brs, 1H), 1.46-1.23 (m, 6H), 0.88 (t, J=6.6 Hz, 3H); ¹³C NMR (150 MHz, CDCl₃) δ 146.36, 145.14, 133.27, 131.25, 129.15, 122.29, 114.34, 111.50, 68.20, 56.01, 37.94, 31.94, 25.31, 22.74, 14.13. HRMS m/z calculated for C₁₅H₂₂O₃[M-H]⁻: 249.1496; found: 249.1523. >95% purity (as determined by RP-HPLC, method C, t_{R}=11.09 min, method H, t_{R}=8.78 min).

### 1-(4-((tert-Butyldimethylsilyl)oxy)-3-methoxyphenyl)octan-3-ol (26)

Compound 26 was prepared as a colorless oil with Compound 22a (33.0 mg, 0.091 mmol) in 94% yield as described above in General Preparation Method D. R_{f}=0.60 (Hexane/EtOAc=5:1, v/v). ¹H NMR (600 MHz, CDCl₃) δ 6.75 (d, J=8.0 Hz, 1H), 6.69 (d, J=2.0 Hz, 1H), 6.64 (dd, J=2.0 and 8.0 Hz, 1H), 3.79 (s, 3H), 3.65-3.57 (m, 1H), 2.75-2.66 (m, 1H), 2.64-2.55 (m, 1H), 1.81-1.66 (m, 2H), 1.54-1.38 (m, 3H), 1.38-1.22 (m, 6H), 0.99 (s, 9H), 0.87 (t, J=7.2 Hz, 3H), 0.14 (s, 6H); ¹³C NMR (150 MHz, CDCl₃) δ 150.75, 143.04, 135.66, 120.72, 120.41, 112.50, 71.50, 55.50, 39.22, 37.59, 31.89, 31.81, 25.76, 25.32, 22.65, 18.45, 14.06, −4.63.

1-(4-Hydroxy-3-methoxyphenyl)octan-3-one (27) (Formula 27)

Compound 27 was prepared as a white oil with Compound 26 (32.0 mg, 0.087 mmol) in 94% yield as described above in General Preparation Method D. R_{f}=0.21 (Hexane/EtOAc=5:1, v/v). ¹H NMR (600 MHz, CDCl₃) δ 6.83 (d, J=7.9 Hz, 1H), 6.72-6.67 (m, 2H), 5.50 (brs, 1H), 3.87 (s, 3H), 3.65-3.59 (m, 1H), 2.77-2.69 (m, 1H), 2.65-2.55 (m, 1H), 1.82-1.66 (m, 2H), 1.53-1.37 (m, 4H), 1.37-1.22 (m, 6H), 0.89 (t, J=7.2 Hz, 3H); ¹³C NMR (150 MHz, CDCl₃) δ 146.41, 143.68, 134.13, 120.90, 114.26, 110.99, 71.52, 55.88, 39.37, 37.59, 31.89, 31.80, 25.30, 22.64, 14.05. HRMS m/z calculated for C₁₅H₂₄O₃[M-H]⁻: 251.1652. found: 251.1683. >95% purity (as determined by RP-HPLC, method C, t_{R}=5.37 min, method G, t_{R}=29.83 min).

### 1-(3,4-Difluorophenyl)hex-1-yn-3-ol (33a) (Formula 33)

Compound 33a was prepared as a colorless oil in 88% yield as described in the method of synthesizing Compound 22a, except that Compound 30 (50.0 mg, 0.240 mmol) was used, instead of Compound 20. R_{f}=0.25 (Hexane/EtOAc=10:1, v/v). ¹H NMR (600 MHz, CDCl₃) δ 7.26-7.20 (m, 1H), 7.18-7.13 (m, 1H), 7.12-7.05 (m, 1H), 4.59 (t, J=6.7 Hz, 1H), 2.11 (brs, 1H), 1.83-1.72 (m, 2H), 1.59-1.49 (m, 2H), 0.98 (t, J=7.2 Hz, 3H); ¹³C NMR (150 MHz, CDCl₃) δ 151.46, 151.37, 150.78, 150.69, 149.79, 149.71, 149.13, 149.04, 135.49, 128.34, 128.31, 128.30, 128.27, 120.69, 120.56, 119.56, 119.53, 119.51, 119.48, 117.48, 117.36, 90.77, 90.75, 82.73, 62.63, 39.83, 18.48, 13.74. HRMS m/z calculated for C₁₂H₁₂F₂O [M-H]⁻: 209.0783. found: 209.0820. >95% purity (as determined by RP-HPLC, method E, t_{R}=5.43 min, method I, t_{R}=4.90 min).

### 1-(3,4-Difluorophenyl)hept-1-yn-3-ol (34a) (Formula 34)

Compound 34a was prepared as a colorless oil in 80% yield as described in the method of synthesizing Compound 22a, except that Compound 31 (17.0 mg, 0.076 mmol) was used, instead of Compound 20. R_{f}=0.29 (Hexane/EtOAc=10:1, v/v). ¹H NMR (600 MHz, CDCl₃) δ 7.26-7.21 (m, 1H), 7.18-7.14 (m, 1H), 7.10 (q, J=8.4 Hz, 1H), 4.57 (dd, J=6.4 and 12.3 Hz, 1H), 1.88 (d, J=5.3 Hz, 1H), 1.85-1.73 (m, 2H), 1.55-1.43 (m, 2H), 1.43-1.34 (m, 2H), 0.94 (t, J=7.2 Hz, 3H); ¹³C NMR (150 MHz, CDCl₃) δ 151.39, 150.79, 150.71, 149.81, 149.72, 149.15, 128.36, 128.33, 128.31, 128.29, 120.71, 120.59, 119.56, 119.50, 117.50, 117.38, 90.76, 82.75, 62.89, 37.50, 27.34, 22.38, 14.01. HRMS m/z calculated for C₁₃H₁₄F₂O [M-H]⁻: 223.0940. found: 223.0966. >95% purity (as determined by RP-HPLC, method E, t_{R}=7.35 min, method I, t_{R}=6.77 min).

### 1-(3,4-Difluorophenyl)oct-1-yn-3-ol (35a) (Formula 35)

Compound 35a was prepared as a colorless oil in 86% yield as described in the method of synthesizing Compound 22a, except that Compound 32 (58.0 mg, 0.245 mmol) was used, instead of Compound 20. R_{f}=0.35 (Hexane/EtOAc=10:1, v/v). ¹H NMR (600 MHz, CDCl₃) δ 7.26-7.20 (m, 1H), 7.18-7.13 (m, 1H), 7.09 (q, J=8.4 Hz, 1H), 4.57 (dd, J=6.4 and 12.3 Hz, 1H), 2.00 (d, J=5.6 Hz, 1H), 1.86-1.71 (m, 2H), 1.57-1.43 (m, 2H), 1.40-1.21 (m, 4H), 0.91 (t, J=7.2 Hz, 3H); ¹³C NMR (150 MHz, CDCl₃) δ 151.38, 150.79, 150.70, 149.80, 149.71, 149.14, 149.05, 128.35, 128.32, 128.31, 128.28, 120.70, 120.57, 119.56, 119.54, 119.51, 119.49, 117.49, 117.37, 90.78, 82.74, 62.89, 37.74, 31.45, 24.87, 22.56, 14.00. HRMS m/z calculated for C₁₄H₁₆F₂O [M-H]⁻: 237.1096. found: 237.1138. >95% purity (as determined by RP-HPLC, method E, t_{R}=10.34 min, method I, t_{R}=9.96 min).

### General Preparation Method E for Compounds 22b, 22c, 33b, 34b, 35b, 33c, 34c, and 35c

A 0.1 M ynone compound solution in 2-propanol as a solvent was added to KOH and a catalyst in the ratio of 200:1:1.2 (ynone:catalyst:KOH). The reaction mixture was stirred (generally, for 4 hours) until a TLC analysis result showed a complete conversion of a starting material to a product, and concentrated under reduced pressure. A residue was purified by silica gel column chromatography, thereby obtaining stereoselective compounds. By using a chiral column (CHIRALPAK IG, 10% ethanol in hexane), an enantiomeric excess (ee) value was determined based on a chiral HPLC analysis result.

### (R)-1-(4-((tert-Butyldimethylsilyl)oxy)-3-methoxyphenyl)oct-1-yn-3-ol (22b)

Compound 22b was prepared as a colorless oil with Compound 20 (150 mg, 0.416 mmol), (R,R)-TsDPEN(mesitylene) (RuCl; 1.29 mg, 2.08 μmol) and KOH (0.138 mg, 2.50 μmol) in 2-propanol (4.16 mL) in 88% yield as described in General Preparation Method E. A residue was purified by silica gel column chromatography (hexane/EtOAc=12:1, v/v), thereby obtaining Compound 22b. R_{f}=0.59 (Hexane/EtOAc=4:1, v/v). ¹H NMR (600 MHz, CDCl₃) δ 6.94-6.92 (m, 2H), 6.77 (d, J=7.9 Hz, 1H), 4.58 (q, J=6.4 Hz, 1H), 3.79 (s, 3H), 1.90 (d, J=5.5 Hz, 1H), 1.81-1.76 (m, 2H), 1.53-1.50 (m, 2H), 1.36-1.33 (m, 4H), 0.98 (s, 9H), 0.91 (t, J=7.0 Hz, 3H), 0.15 (s, 6H); ¹³C NMR (150 MHz, CDCl₃) δ 150.66, 145.84, 124.95, 120.90, 115.68, 115.30, 88.71, 85.02, 63.09, 55.44, 37.97, 31.51, 25.68, 24.95, 22.60, 18.47, 14.04, −4.65.

### (S)-1-(4-((tert-Butyldimethylsilyl)oxy)-3-methoxyphenyl)oct-1-yn-3-ol (22c)

Compound 22c was prepared as a colorless oil with Compound 20 (150 mg, 0.416 mmol), (S,S)-TsDPEN(mesitylene) (RuCl; 1.29 mg, 2.08 μmol) and KOH (0.138 mg, 2.50 μmol) in 2-propanol (4.16 mL) in 68% yield as described above in General Preparation Method E. A residue was purified by silica gel column chromatography (Hexane/EtOAc=12:1, v/v), thereby obtaining Compound 22c. R_{f}=0.59 (Hexane/EtOAc=4:1, v/v). ¹H NMR (600 MHz, CDCl₃) δ 6.94-6.92 (m, 2H), 6.77 (d, J=7.9 Hz, 1H), 4.58 (t, J=6.5 Hz, 1H), 3.79 (s, 3H), 1.91 (s, 1H), 1.81-1.76 (m, 2H), 1.53-1.49 (m, 2H), 1.35-1.34 (m, 4H), 0.98 (s, 9H), 0.91 (t, J=7.0 Hz, 3H), 0.15 (s, 6H); ¹³C NMR (150 MHz, CDCl₃) δ 150.66, 145.84, 124.95, 120.90, 115.68, 115.31, 88.71, 85.01, 63.08, 55.44, 37.97, 31.51, 25.68, 24.95, 22.60, 18.47, 14.04, −4.65.

### (R)-1-(3,4-Difluorophenyl)hex-1-yn-3-ol (33b) (Compound 33)

Compound 33b was prepared as a colorless oil with Compound 30 (100 mg, 0.48 mmol), (R,R)-TsDPEN(mesitylene) (RuCl; 1.49 mg, 2.40 μmol) and KOH (0.16 mg, 2.90 μmol) in 2-propanol (4.8 mL) in 86% yield as described in General preparation method E. A residue was purified by silica gel column chromatography (hexane/EtOAc=20:1, v/v), thereby obtaining Compound 33b. R_{f}=0.25 (Hexane/EtOAc=10:1, v/v). ¹H NMR (600 MHz, CDCl₃) δ 7.25-7.19 (m, 1H), 7.18-7.13 (m, 1H), 7.12-7.04 (m, 1H), 4.59 (t, J=2.9 Hz, 1H), 2.18 (brs, 1H), 1.83-1.72 (m, 2H), 1.58-1.49 (m, 2H), 0.98 (t, J=7.2 Hz, 3H); ¹³C NMR (150 MHz, CDCl₃) δ 151.45, 151.37, 150.78, 150.69, 149.79, 149.70, 149.12, 149.04, 128.34, 128.31, 128.29, 128.27, 120.68, 120.56, 119.56, 119.53, 119.51, 119.48, 117.48, 117.36, 90.77, 90.76, 82.72, 62.62, 39.83, 18.49, 13.74. HRMS m/z calculated for C₁₂H₁₂F₂O [M-H]⁻: 209.0783. found: 209.0811. >95% purity (as determined by RP-HPLC, method E, t_{R}=5.42 min, method I, t_{R}=4.86 min).

### (R)-1-(3,4-Difluorophenyl)hept-1-yn-3-ol (34b) (Formula 34)

Compound 34b was prepared as a colorless oil with Compound 31 (100 mg, 2.30 mmol), (R,R)-TsDPEN(mesitylene) (RuCl; 1.40 mg, 2.30 μmol) and KOH (0.15 mg, 2.70 μmol) in 2-propanol (4.5 mL) in 82% yield as described in General preparation method E. A residue was purified by silica gel column chromatography (hexane/EtOAc=20:1, v/v), thereby obtaining Compound 34b. R_{f}=0.29 (Hexane/EtOAc=10:1, v/v). ¹H NMR (600 MHz, CDCl₃) δ 7.26-7.19 (m, 1H), 7.19-7.13 (m, 1H), 7.12-7.05 (m, 1H), 4.57 (t, J=2.9 Hz, 1H), 2.18 (brs, 1H), 1.85-1.74 (m, 2H), 1.53-1.45 (m, 2H), 1.42-1.34 (m, 2H), 0.94 (t, J=7.2 Hz, 3H); ¹³C NMR (150 MHz, CDCl₃) δ 150.78, 150.69, 149.79, 149.70, 149.12, 149.04, 128.34, 128.32, 128.30, 128.28, 120.69, 120.56, 119.54, 119.49, 117.48, 117.36, 90.80, 82.71, 62.85, 37.48, 27.35, 22.37, 13.99. HRMS m/z calculated for C₁₃H₁₄F₂O [M-H]⁻: 223.0940; found: 223.0973. >95% purity (as determined by RP-HPLC, method E, t_{R}=7.32 min, method I, t_{R}=6.64 min).

### (R)-1-(3,4-Difluorophenyl)oct-1-yn-3-ol (35b) (Formula 35)

Compound 35b was prepared as a colorless oil with Compound 32 (100 mg, 0.42 mmol), (R,R)-TsDPEN(mesitylene) (RuCl; 1.31 mg, 2.10 μmol) and KOH (0.14 mg, 2.50 μmol) in 2-propanol (4.2 mL) in 80% yield as described above in General preparation method E. A residue was purified by silica gel column chromatography (hexane/EtOAc=20:1, v/v), thereby obtaining Compound 35b. R_{f}=0.35 (Hexane/EtOAc=10:1, v/v). ¹H NMR (600 MHz, CDCl₃) δ 7.25-7.21 (m, 1H), 7.20-7.15 (m, 1H), 7.14-7.03 (m, 1H), 4.57 (dd, J=6.0 and 10.8 Hz, 1H), 2.28 (d, J=4.2 Hz, 1H), 1.89-1.72 (m, 2H), 1.58-1.42 (m, 2H), 1.38-1.33 (m, 4H), 0.90 (t, J=7.2 Hz, 3H); ¹³C NMR (150 MHz, CDCl₃) δ 151.45, 151.36, 150.77, 150.69, 149.78, 149.70, 149.12, 149.04, 128.33, 128.31, 128.29, 128.27, 120.68, 120.55, 119.58, 119.55, 119.53, 119.50, 117.47, 117.35, 90.82, 82.70, 62.85, 37.72, 31.44, 24.88, 22.56, 13.98. HRMS m/z calculated for C₁₄H₁₆F₂O [M-H]⁻: 237.1096. found: 237.1139. >95% purity (as determined by RP-HPLC, method E, t_{R}=10.26 min, method I, t_{R}=11.03 min).

### (S)-1-(3,4-Difluorophenyl)hex-1-yn-3-ol (33c) (Formula 33)

Compound 33c was prepared as a colorless oil with Compound 30 (100 mg, 0.48 mmol), (S,S)-TsDPEN(mesitylene) (RuCl; 1.49 mg, 2.40 μmol) and KOH (0.16 mg, 2.90 μmol) in 2-propanol (4.8 mL) in 86% yield as described in General preparation method E. A residue was purified by silica gel column chromatography (Hexane/EtOAc=20:1, v/v), thereby obtaining Compound 33c. R_{f}=0.25 (Hexane/EtOAc=10:1, v/v). ¹H NMR (600 MHz, CDCl₃) δ 7.26-7.18 (m, 1H), 7.18-7.12 (m, 1H), 7.12-7.03 (m, 1H), 4.59 (t, J=2.9 Hz, 1H), 2.39 (brs, 1H), 1.84-1.72 (m, 2H), 1.59-1.47 (m, 2H), 0.98 (t, J=7.2 Hz, 3H); ¹³C NMR (150 MHz, CDCl₃) δ 151.44, 151.36, 150.76, 150.68, 149.77, 149.69, 149.12, 149.03, 128.33, 128.30, 128.28, 128.26, 120.67, 120.54, 119.57, 119.54, 119.52, 119.49, 117.46, 117.35, 90.79, 82.71, 82.69, 62.59, 39.81, 18.49, 13.72. HRMS m/z calculated for C₁₂H₁₂F₂O [M-H]⁻: 209.0783. found: 209.0816. >95% purity (as determined by RP-HPLC, method E, t_{R}=5.42 min, method I, t_{R}=4.88 min).

### (S)-1-(3,4-Difluorophenyl)hept-1-yn-3-ol (34c) (Formula 34)

Compound 29c was prepared as a colorless oil with Compound 31 (100 mg, 0.45 mmol), (S,S)-TsDPEN(mesitylene) (RuCl; 1.4 mg, 2.30 μmol) and KOH (0.15 mg, 2.70 μmol) in 2-propanol (4.5 mL) in 82% yield as described in General preparation method E. A residue was purified by silica gel column chromatography (Hexane/EtOAc=20:1, v/v), thereby obtaining Compound 34c. R_{f}=0.29 (Hexane/EtOAc=10:1, v/v). ¹H NMR (600 MHz, CDCl₃) δ 7.26-7.19 (m, 1H), 7.19-7.12 (m, 1H), 7.12-7.04 (m, 1H), 4.57 (t, J=2.9 Hz, 1H), 2.37 (brs, 1H), 1.86-1.72 (m, 2H), 1.54-1.43 (m, 2H), 1.43-1.34 (m, 2H), 0.93 (t, J=7.2 Hz, 3H); ¹³C NMR (150 MHz, CDCl₃) δ 151.44, 151.36, 150.77, 150.68, 149.77, 149.69, 149.12, 149.03, 128.33, 128.31, 128.29, 128.27, 120.67, 120.55, 119.58, 119.55, 119.53, 119.50, 117.46, 117.34, 90.83, 90.82, 82.69, 62.82, 37.47, 27.35, 22.37, 13.97. HRMS m/z calculated for C₁₃H₁₄F₂O [M-H]⁻: 223.0940. found: 223.0971. >95% purity (as determined by RP-HPLC, method E, t_{R}=7.32 min, method I, t_{R}=6.72 min).

### (S)-1-(3,4-Difluorophenyl)oct-1-yn-3-ol (35c) (Formula 35)

Compound 35c was prepared as a colorless oil with Compound 32 (100 mg, 0.42 mmol), (S,S)-TsDPEN(mesitylene) (RuCl; 1.31 mg, 2.10 μmol) and KOH (0.14 mg, 2.50 μmol) in 2-propanol (4.2 mL) in 82% yield as described in General preparation method E. A residue was purified by silica gel column chromatography (hexane/EtOAc=20:1, v/v), thereby obtaining Compound 35c. R_{f}=0.35 (Hexane/EtOAc=10:1, v/v). ¹H NMR (600 MHz, CDCl₃) δ 7.26-7.19 (m, 1H), 7.19-7.12 (m, 1H), 7.12-7.04 (m, 1H), 4.57 (t, J=2.9 Hz, 1H), 2.31 (brs, 1H), 1.85-1.72 (m, 2H), 1.56-1.44 (m, 2H), 1.39-1.28 (m, 4H), 0.90 (t, J=7.2 Hz, 3H); ¹³C NMR (150 MHz, CDCl₃) δ 151.44, 151.36, 150.77, 150.68, 149.78, 149.69, 149.12, 149.04, 128.33, 128.31, 128.29, 128.26, 120.67, 120.55, 119.58, 119.55, 119.53, 119.50, 117.47, 117.35, 90.82, 82.70, 62.85, 37.72, 31.44, 24.88, 22.56, 13.97. HRMS m/z calculated for C₁₄H₁₆F₂O [M-H]⁻: 237.1096. found: 237.1137. >95% purity (as determined by RP-HPLC, method E, t_{R}=10.31 min, method I, t_{R}=9.79 min).

[Chiral HPLC Analysis]

The enantiomeric excess (ee) of a compound was determined by chiral HPLC analysis using a chiral column (CHIRALPAK IG (4.6 i.d×250 mm). The chromatography analysis was performed by high-performance liquid chromatography (Agilent 1260 series) with the following two methods A and B. Method A was performed in a mixture of 10% ethanol and hexane (v/v) in an isocratic manner for 30 minutes or more at a flow rate of 1 mL/min. Auto-sampler and column compartment temperatures were set to 25° C., UV detection was performed at a wavelength of 254 nm, and 5 μL of a sample was injected. Method B was performed in a mixture of 1% ethanol and hexane (v/v) in an isocratic manner for 30 minutes or more at a flow rate of 0.8 m/min. Auto-sampler and column compartment temperatures were set to 25° C., UV detection was performed at a wavelength of 254 nm, and 3 μL of a sample was injected.

[QS Reporter Strain Assay]

Escherichia coli (E. coli) DH5a includes LuxR homolog SdiA detecting endogenous AHL synthesized by other microbial species, particularly, BHL.

For RhlR reporter strain analysis, the present invention used pJN105R (RhlR expression plasmid), anspSC11 (RhlA::lacZ fusion plasmid) and an sdiA variant co-transformed with the two plasmids. 1% of an overnight-cultured reporter strain (OD 595 nm=1.0) supplemented with 10 μg/mL gentamicin and 50 μg/mL ampicillin was cultured in a Luria-Bertani (LB) medium (Difco, Detroit, Mich., USA) with a positive control or a compound (0-1000 μM) and BHL (0-10 μM) (Sigma-Aldrich) at 37° C. for 2 hours. 0.4% arabinose (Sigma-Aldrich) was reacted with the resulting product at 37° C. for 1.5 hours. β-galactosidase activity was evaluated using a Tropix plus kit (Applied Biosystems, CA, USA), and OD 595 nm and luminescence were measured using a VICTOR x5 multimode plate reader (PerkinElmer, Waltham, Mass., USA). Relative RhlR activity was quantified by measuring luminescence at OD 595 nm. In addition, for LasR reporter strain analysis, the present invention used pJN105L (LasR expression plasmid) and pSC11 (lasI::lacZ fusion plasmid), E. coli DH5a co-transformed with the two plasmids, and OdDHL (Sigma-Aldrich).

In addition, for PqsR reporter strain analysis, the present invention used a PqsR expression plasmid and pSC11 (PqsA::lacZ fusion plasmid), E. coli DH5a co-transformed with the two plasmids, and PQS (Sigma-Aldrich).

[Static Biofilm Formation Assay]

Pseudomonas aeruginosa (P. aeruginosa) (OD 595 nm=1.0) was cultured overnight, and diluted in an AB medium (1:20; 300 mM NaCl, 50 mM MgSO₄, 0.2% vitamin-free casamino acids, 10 mM potassium phosphate, 1 mM L-arginine, and 1% glucose, pH 7.5) together with a positive control or compound (0100 μM) in a borosilicate bottle. The resulting mixture was incubated without shaking at 37° C. for 24 hours, and to measure growth inhibition activity. OD 595 nm of the suspended culture was measured using a multimode plate reader. Biofilm cells attached to the bottle were washed twice with phosphate-buffered saline (PBS; 137 mM NaCl, 2.7 mM KCl, 10 mM Na₂HPO₄, and 2 mM KH₂PO₄, pH 7.2). Afterward, the remaining biofilm cells were stained with 0.1% crystal violet for 10 minutes, and to remove the unbound crystal violet, washed with deionized water. The crystal violet not binding to the biofilm cells was eluted with 100% ethanol, and the OD of the eluted ethanol sample was measured at 545 nm using a multimode plate reader.

[Dynamic Biofilm Formation Assay]

Glass slides were placed in a drip-flow reactor, and an AB medium containing 5% P. aeruginosa (OD 595 nm=1.0) was added along with a positive control or compound (0 to 10 μM) and the reactor was continuously operated using peristaltic pumps (Masterflex C/L tubing pumps, Cole-Parmer, Ill., USA) at 0.3 ml/min. After the above-described process was carried out at 37° C. for 48 hours, unattached biofilm cells were removed with PBS. The remaining biofilm cells were stained with DAPI (Sigma-Aldrich) for 10 minutes, and observed by confocal laser scanning microscopy (CLSM; Carl Zeiss LSM700, Jena, Germany). The CLSM image of the biofilm cells was obtained with a 20× objective lens (W NAchroplanX20/0.5W (DIC) M27) under blue fluorescence light (excitation wavelength: 350 nm, emission wavelength: 470 nm) using a Z-stack mode of the Zen 2011 program. The volume and thickness of the biofilm were measured using Comstat2 of the ImageJ program based on a CLSM image.

[EPS Analysis]

EPS analysis was carried out with the biofilm cells attached to the slides after the operation of the drip-flow reactor. The biofilm cells that were centrifuged were re-suspended with 0.01M KCl, and for the analysis of carbohydrates and proteins, the cells were disrupted with a sonicator (VCX 750, SONICS, Newtown, Conn., USA) for 5 seconds at a level of 3.5 Hz, followed by stopping for 5 seconds, which was repeated 4 times. A supernatant of the cell-disrupted suspension was filtered through a 0.22 μm membrane filter (Millex filter, Carl Roth, Karlsruhe, Germany). For protein analysis, a mixture containing 40 μL of the filtrate and 200 μL of a Lowry reagent (L3540, Sigma-Aldrich) was incubated at room temperature for 10 minutes. Afterward, 20 μL of a Folin-Ciocalteu reagent (Sigma-Aldrich) was added and incubated at room temperature for 30 minutes, followed by measuring OD at 750 nm using a multimode plate reader. A protein amount was quantified at OD 750 nm or OD 595 nm. For carbohydrate analysis, a mixture containing 50 μL of the filtrate and 150 μL of 99.9% sulfuric acid was incubated at room temperature for 30 minutes. Afterward, 5% phenol was added, followed by incubation at 90° C. for 5 minutes. OD was measured at 490 nm using a multimode plate reader. The amount of carbohydrates was quantified at OD 490 nm or OD 595 nm.

[Rhamnolipid Production Assay]

P. aeruginosa (OD 595 nm=1.0) cultured overnight was inoculated in an AB medium (1:100) with a positive control or compound (0100 μM), and incubated using a shaking culture for 24 hours at 37° C. The cell culture was centrifuged for 5 minutes at 4° C. and 12,000 g. Rhamnolipids were extracted twice by mixing 200 μL of a supernatant and 400 μL of 100% diethyl ether (JUNSEI, Tokyo, Japan). The supernatant was transferred to a new tube and evaporated. A dried sample was eluted with 20 μL of deionized water, and reacted with 180 μL of an Orcinal solution (0.19% Orcinal (Sigma-Aldrich) in 53% H₂SO₄). The reaction mixture was heated for 30 minutes at 80° C., and cooled for 15 minutes at room temperature. The amount of rhamnolipids was measured at OD 421 nm, and normalized in the cell culture solution at OD 595 nm using a multimode plate reader.

[Pyocyanin Production Assay]

P. aeruginosa cultured overnight with a positive control or compound (0-10 μM) was inoculated in an AB medium, incubated for 24 hours at 37° C., and centrifuged for 10 minutes at 10,000 rpm. To extract pyocyanin, the supernatant was reacted with 50% trifluoroacetic acid (TFA, Sigma-Aldrich) for 1 hour at 37° C. The reacted supernatant was centrifuged for 10 minutes at 10,000 rpm, and filtered through a 0.22-μm filter. The filtrate solution was analyzed using a 1260 Infinity II Prep-HPLC System (Agilent Technologies, Santa Clara, Calif., USA) and an EC-C18 column (4.6×150 mm, Agilent Technologies). Specific HPLC conditions are as follows: mobile phase deionized water; TFA (99:1); injection volume:10 μL; temperature: 25° C.; and flow rate: 0.5 mL/min. The retention time of pyocyanin (Sigma-Aldrich) is 20 minutes. To measure a pyocyanin amount, when the retention time was 20 minutes, the height of an analysis peak was detected.

[Experiment for Mortality of T. molitor Larvae]

The mortality of Tenebrio molitor (T. molitor) larvae was measured with a positive control or compound (0-10 μM) by the method used as in previous research. Overnight-cultured P. aeruginosa (OD 595 nm=1.0) was centrifuged for 10 minutes at 10,000 rpm. The supernatant was filtered through a 0.22-μm filter, and injected into the larvae using a syringe needle. The larvae were stored in a petri dish for 20 days at 25° C. The mortality of the larvae was measured every 5 days.

[RT-qPCR Analysis]

P. aeruginosa (OD 595 nm=1.0) overnight cultured with a positive control or compound (0-10 μM) was diluted (1:20) in an AB medium in a borosilicate bottle. Afterward, after culture for 24 hours at 37° C., biofilm cells were collected for RNA extraction. Total RNA was extracted using a TRI reagent (Molecular Research Center, OH, USA) according to the manufacturer's instruction. To quantify and compare QS-related gene expression levels, RT-qPCR was performed. For RT-qPCR, a SYBR Premix Ex Taq™ (Takara, Shiga, Japan), a CFX-96 real-time system (Bio-Rad, Hercules, Calif. USA), and a QS-related gene primer set were used. The thermal profile of the RT-qPCR is as follows: 40 cycles of initial denaturation for 10 seconds at 95° C., denaturation for 10 seconds at 95° C., annealing for 10 seconds at 60° C., and extension for 34 seconds at 63° C. A fluorescent signal intensity was collected at the end of the elongation step.

[In Silico Docking Studies]

An RhlR homology model was constructed with 3-oxo-C6-homoserine lactone (PDB code 4Y15) based on the crystal structure of SdiA in a complex. The protein sequence of RhlR, in an FASTA format, was obtained from the NCBI protein database (http://ncbi.nlm.nih.gov/protein). The RhlR homology model was constructed using SWISS-MODEL (https://swissmodel.expasy.org). All of the final compounds were prepared in a 3D structure using Chem3D Pro (ver 12.0), and the compounds were stored as an .sdf file. A ‘Sanitize’ preparation protocol in SYBYL-X 2.1.1 (Tripos Inc., St Louis) was applied to ligand preparation and optimization processes. The prepared final compounds were docked in the RhlR homology models using a Surflex-Dock GeomX module of SYBYL-X 2.1.1. The protein minimization for molecular docking was performed by a “POWELL” method with a Tripos' Force Field setting. The initial optimization selection was set to none. As defined in the “Residues” method, based on amino acids (Tyr43, Val60, Tyr64, Trp68, Asp81, Ile84, Ser135; radius setting: 4.0; the amino acids were selected based on an active site of SdiA and the template protein of the RhlR homology model.) selected to guide a docking position, other docking parameters were maintained at their initial values.

[Statistical Analysis]

The P value was evaluated by the Student's t test (SigmaPlot version 10, Systat Software Inc., San Jose, Calif., USA).

[Results and Conclusion]

### [Experimental Example 1]: RhlR Antagonistic Activity According to Alkyl Chain Length of Gingerol

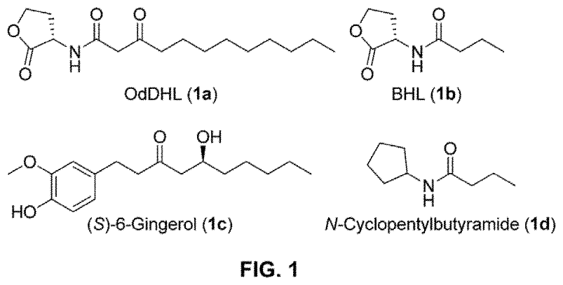

To develop a pure RhlR antagonist, in the present invention, the relative RhlR activity of gingerol analogs according to the lengths of various alkyl chains including 4-gingerol to 10-gingerol was screened (Scheme 1). In the present invention, for in vitro biological evaluation, DMSO was used as a negative control, and (S)-6-gingerol (Compound 1c) and N-cyclopentylbutyramide (Compound 1d) were used as positive controls (FIG. 1). A gingerol with a shorter alkyl chain has a higher affinity to RhlR and stronger RhlR antagonistic activity. All gingerols of the present invention showed a very low RhlR agonistic activity of less than 4%. Since the 4-gingerol (Compound 2) having the shortest alkyl chain in the present invention has the most similar chemical structure to BHL, and competes with BHL in binding to RhlR, the highest relative RhlR antagonistic activity of 54% was exhibited (Table 2).

TABLE 2

| | Relative RhlR activity | | Relative RhlR activity | | |
|---|---|---|---|---|---|
| Com- | | | Com- | | |
| pound^{a} | Antagonism^{b} | Agonism [symbol] | pound | Antagonism | Agonism |
| 1c | 75** | 3** | 11p | 72** | 2** |
| 1d | 54** | 4** | 11q | 43** | 1** |
| 2 | 54** | 3** | 11r | 74** | 3** |
| 3 | 62** | 2** | 12a | 69** | 4** |
| 4 | 76** | 4** | 12b | 88* | 4** |
| 5 | 83** | 3** | 12c | 90* | 2** |
| 6 | 90* | 2** | 12d | 64** | 3** |
| 7 | 41** | 2** | 12e | 81** | 2** |
| 8 | 36** | 2** | 12f | 71** | 2** |
| 11a | 65** | 4** | 12g | 51** | 2** |
| 11b | 93* | 4** | 12h | 69** | 2** |
| 11c | 94 | 2** | 12i | 76** | 1** |
| 11d | 51** | 1** | 12j | 74** | 1** |
| 11e | 78** | 2** | 12k | 47** | 2** |
| 11f | 55** | 1** | 12l | 94 | 1** |
| 11g | 33** | 1** | 12m | 73** | 1** |
| 11h | 64** | 1** | 12n | 98 | 1** |
| 11i | 65** | 2** | 12o | 49** | 1** |
| 11j | 53** | 2** | 12p | 82** | 3** |
| 11k | 43** | 3** | 12q | 79** | 1** |
| 11l | 69** | 1** | 12r | 85** | 3** |
| 11m | 60** | 2** | 13a | 51** | 4** |
| 11n | 91 | 1** | 13b | 41** | 2** |
| 11o | 20** | 2** | 14a | 55** | 4** |
| | | | 14b | 49** | 2** |
| | | | 15a | 55** | 5** |
| | | | 15b | 41** | 2** |
| | | | 16a | 61** | 5** |
| | | | 21 | 29** | 3** |
| | | | 23a | 32** | 3** |
| | | | 23b | 27** | 3** |
| | | | 23c | 34** | 3** |
| | | | 25 | 79** | 3** |
| | | | 27 | 45** | 2** |
| | | | 30 | 12** | 4** |
| | | | 31 | 16** | 4** |
| | | | 32 | 17** | 3** |
| | | | 33a | 32** | 3** |
| | | | 33b | 25** | 3** |
| | | | 33c | 38** | 3** |
| | | | 34a | 71** | 4** |
| | | | 34b | 67** | 3** |
| | | | 34c | 64** | 2** |
| | | | 35a | 74** | 3** |
| | | | 35b | 72** | 3** |
| | | | 35c | 67** | 4** |

[symbol]

indicates data missing or illegible when filed

To establish the structure-activity relationship (SAR) of 4-gingerol as an RhlR antagonist, in the present invention, the following structural modification was performed.

### [Experimental Example 2]: RhlR Antagonistic Activity According to Modification of Phenyl Ring Substituent of Gingerol Derivative

To examine the effect of a phenyl ring substituent on RhlR antagonistic activity, 3′-OMe and 4′-OH groups of a phenyl group in 4-gigerol were replaced with various functional groups (—F, —Cl, —OH, —OMe, —OEt, -Me, and —N(CH₃)₂). As shown in Scheme 1, Compounds 10a-10r were synthesized by treating benzaldehyde with 10% NaOH under acetone and ethanol (or water) at 25° C. Compounds 11a-11r were obtained in 24 to 60% yields by reacting the Compounds 10a-10r with diisoproprylamide (LDA) at −78° C. and adding n-butanol.

Compound 11b that does not have a substituent in a phenyl ring completely lost antagonistic activity (93%), confirming that a polar functional group of the phenyl ring is necessary for binding to RhlR. To evaluate the necessity of the 4′-OH and 3′-OMe of a phenyl ring for RhlR antagonistic activity, 4′-OH (Compound 11c) and 3′-OMe (Compound 11d) were removed.

Compound 11c was decreased in the ability to inhibit RhlR compared to Compound 11a (65%), but Compound 11d (51%) had slightly increased RhlR antagonistic activity compared to Compound 11a. Compound 11f with 4′-OMe (55%) had less potent RhlR antagonistic activity compared to Compound 11d, confirming that a polar group present at position 4 is suitable for increasing RhlR-binding ability. Compound 11e having only 3′-OH (78%) had stronger RhlR antagonistic activity than Compound 11c only having 3′-OMe.

To expand the 4-gingerol SAR, single-substituted compounds (Compounds 11g to 11j) and double-substituted compounds (Compound 11k to 11r) were synthesized. Among the single-substituted compounds, Compound 1 Ig having 4′-F had the highest relative RhlR antagonistic activity at 33%. Compound 11j (53%) having 4′-N(CH₃)₂ was more potent than Compound 11a, but Compound 11h (64%) having 4′-Cl and Compound 11i (65%) having 4′-Me were similar to Compound 11a. A similar trend was also observed for the double-substituted compounds. In the present invention, Compound 110 (20%) having 3′4′-di-F showed the most potent RhlR antagonistic activity. Through this, it was confirmed that an F substituent is preferable for the structural modification of a phenyl ring. Compound 11q (43%) having 3′-OMe and 4′-F had a higher antagonistic activity than Compound 11a having 3′-OMe and 4′-OH, but Compound 11r (74%) having 3′-F and 5′-OMe was less potent than Compound 11a. From the above result, it was confirmed that a small polar group is needed at position 4. Compound 11p having hydrophobic -Me groups at the positions 3 and 4 (72%) showed decreased RhlR inhibitory activity. Compound 11n (91%) having 3′-OEt and 4′-OH had dramatically reduced RhlR antagonistic activity. From this result, it was confirmed that the RhlR site interacting with position 3 of the 4-gingerol is very sensitive to the size of a substituent. The SAR results demonstrated that position 4 is related to hydrogen bond interaction with RhlR, whereas position 3 thereof is sensitive to a substituent size.

### [Experimental Example 3]: RhlR Antagonistic Activity According to Rotational Rigidity of Gingerol Derivative

To evaluate the effect of rotational rigidity on RhlR antagonistic activity, compounds having a single bond between a phenyl ring and a carbonyl group (Compounds 12a to 12r) were synthesized. As shown in Scheme 1, Compounds 12a to 12r were obtained in 40 to 93% yields by reducing Compounds 11a to 11r with a hydrogen gas in the presence of 10% Pd/C.

Comparing the compounds having a double bond between a phenyl ring and a carbonyl group (Compounds 11a to 11r) and compounds having a single bond (Compounds 12a to 12r), the compounds having a double bond (Compounds 11a to 11r) showed stronger RhlR antagonistic activity than the compounds having a single bond (Compounds 12a to 12r) regardless of a substituent of a phenyl ring. From the above result, it was confirmed that rotational rigidity between a phenyl ring and a carbonyl ring significantly increases the binding affinity of RhlR. Overall, double-bonded Compound 11o in which F groups are substituted at positions 3 and 4 exhibited the strongest RhlR antagonistic activity and the lowest RhlR agonistic activity (2%), among the compounds in which a phenyl ring is modified.

### [Experimental Example 4]: RhlR Antagonistic Activity According to Removal of p-Hydroxyl Group of Gingerol Derivative

2-heptanone was reacted with vanillin or 3,4-difluorobenzaldehyde under a basic condition for 72 hours at 25° C., thereby obtaining β-hydroxy group-free 4-gingerol derivatives (Compound 13a and 13b) in 30 to 50% yields. Compounds 14a and 14b were obtained in approximately 76% yield by treating α, β-unsaturated carbonyl groups of Compounds 13a and 13b with H₂ and Pd/C through catalytic hydrogenation. Compounds 15a and 15b were obtained in 94% and 90% yields, respectively, by treating the α, β-unsaturated carbonyl groups of Compounds 13a and 13b with NaBH₄ to be reduced to a secondary alcohol. Compound 16a was obtained in 60% yield by reducing Compound 15a to a single bond.

β-Hydroxyl group-free Compound 13b (41%) was more potent than Compound 11a (65%), confirming that the β-hydroxyl group is not necessary for binding to RhlR. The α, β-unsaturated carbonyl analog (Compound 13b) was more potent than the α, β-saturated carbonyl analog (Compound 14b). Compound 15b in which an α,β-unsaturated carbonyl group is reduced to an alcohol (41%) was maintained RhlR-binding affinity. However, the RhlR antagonistic activity of the 3,4-difluorophenyl ring analogs (Compounds 13a to 15a) were almost similar.

### [Experimental Example 5]: RhlR Antagonistic Activity According to Changes in Rotational Rigidity and Absolute Configuration of Gingerol Derivatives

As shown in Scheme 2, a compound having a triple bond was synthesized. Compound 17 was obtained by protecting phenolic OH of vanillin with a tert-butyldimethylsilyl (TBDMS) group. Compound 18, as a dibromo alkene, was obtained in 98% yield by treating Compound 17 with CBr₄ and PPh₃ in the presence of dichloromethane. Compound 19, as a terminal alkyne, was obtained in 96% yield through elimination and lithiation of Compound 18 in the presence of 3 equivalents of n-BuLi. Compound 20 was obtained by reacting Compound 19 with a Weinreb amide. Compound 21, as an alkynyl ketone, was obtained in 94% yield by treating Compound 20 with tetrabutylammoniumfluoride (TBAF). Enantiomerically-enriched alkynyl alcohol analogs (Compounds 23b and 23c) were obtained by treatment of Compound 20 with a chiral catalyst such as RuCl[(R, R)-TsDPEN(mesitylene)] or RuCl[(S, S)-TsDPEN(mesitylene)] and desilylation with TBAF. Compound 23a, as a racemic mixture, was obtained by treatment of Compound 20 with NaBH₄ and desilylation.

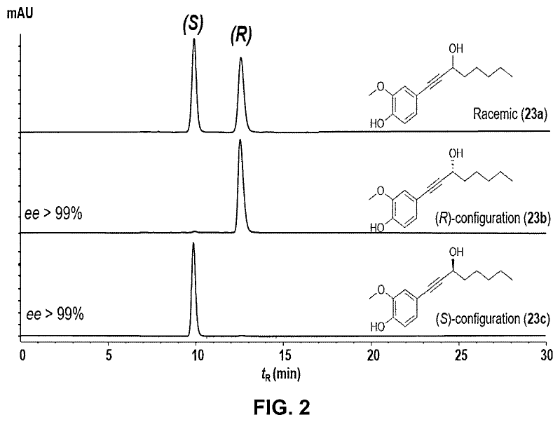

Alkynyl alcohols (Compounds 23a and 23b) were analyzed using chiral reversed phase HPLC. As a result of chiral HPLC, since Compound 23a is a racemate, two distinct peaks were shown in 1:1 ratio at 9.91 minutes and 12.58 minutes. Compound 23b, as an (R)-configuration, was eluted at 12.54 minutes, whereas Compound 23c, as an (S)-configuration, was eluted at 9.88 minutes. Here, an enantiomeric excess (ee) value exceeded 99% (FIG. 2). Compound 25, as a cis isomer, was obtained by synthesizing the Z-selective reduction of Compound 22a, and specifically, treatment of Compound 22a with a Lindlar catalyst and 1,4-benzoquinone in the presence of a hydrogen gas and desilylation with TBAF. Compound 27, as an alkanyl alcohol, was obtained in 94% yield through lithiation and desilylation of Compound 22a.

Compound 21 (29%), as an alkynyl ketone, showed stronger relative RhlR activity than Compound 13b (41%), which is an alkenyl ketone, and Compound 14b (49%), which is an alkanyl ketone (Table 1). Likewise, Compound 23a (32%), as an alkynyl alcohol, showed a stronger relative RhlR activity than Compound 15b (41%), which is an alkene, and Compound 27 (45%), which is an alkane. That is, the relative RhlR activity of alkynyl ketone (Compound 21) or alkynyl alcohol (Compounds 23a to 23c) was less than 34%, which is stronger activity than the conventionally-reported RhlR antagonist (Compound 1d, 54%).

Looking at the effect of absolute configuration on RhlR affinity, Compound 23b (27%), which is an (R)-enantiomer, showed stronger RhlR antagonistic activity than Compound 23c (34%), which is an (S)-enantiomer, and Compound 23a (32%), which is a racemate. The relative RhlR activity of Compound 25, which is a (Z)-alkenyl alcohol, was 79%, which is lower than Compound 15b (41%), which is a (E)-alkenyl alcohol, confirming that the cis-enantiomer is not appropriately located at the active site of RhlR. Overall, from the SAR results, it was confirmed that a carbonyl or hydroxyl group located at a γ-position from a phenyl group is important for binding to RhlR.

### [Experimental Example 6]: RhlR Antagonistic Activity According to Changes in Rotational Rigidity and Absolute Configuration of 3,4-Difluorophenyl Derivatives

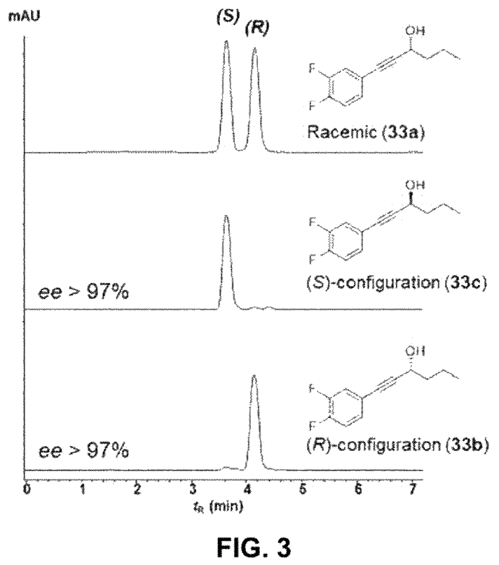

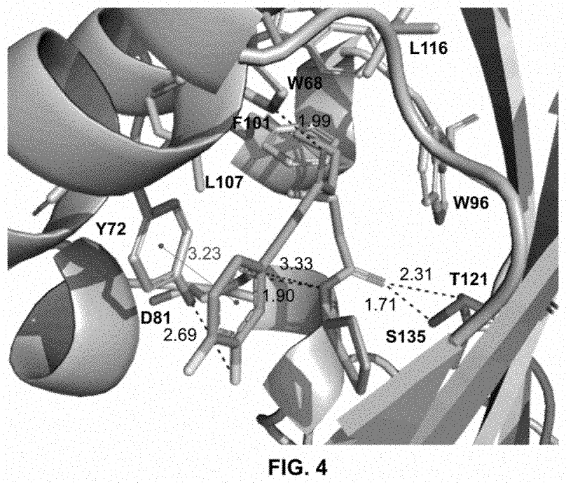

In the present invention, based on the SAR research for 4-gingerol analogs, a 3,4-difluorophenyl analog, which has the most potent RhlR binding activity, was synthesized by phenyl ring substituent modification. As shown in Scheme 3, by the same synthesis method for 4-gingerol analogs, alkynyl ketones (Compounds 30 to 32) and alkynyl alcohols (33a to 33c, 34a to 34c, and 35a to 35c) were prepared using 3,4-difluorophenyl. The purity and enantiomer excess (ee) of the compounds were analyzed using chiral HPLC (FIG. 3).

Alkynyl ketones (Compounds 30 to 32) were more potent than corresponding alkene and alkane compounds with a relative RhlR activity of less than 17% at 100 μM. In addition, the (R)-enantiomers (Compounds 33b to 5b) had higher activity than the corresponding (S)-enantiomers (Compounds 33c to 35c) and racemates (Compounds 33a to 35a). Further, the compounds having a shorter alkyl chain (Compounds 30 and 33a to 33c) had higher activity than the corresponding compounds having a long alkyl chain (Compounds 31, 32, 34a to 34c, and 35a to 35c). The alkynyl ketone having an n-propyl group (Compound 30) had a relative RhlR activity of 12%, and had the highest activity among the 3,4-difluorophenyl derivatives, which is much stronger activity than the conventionally-reported antagonist (Compound 1d, 54%).

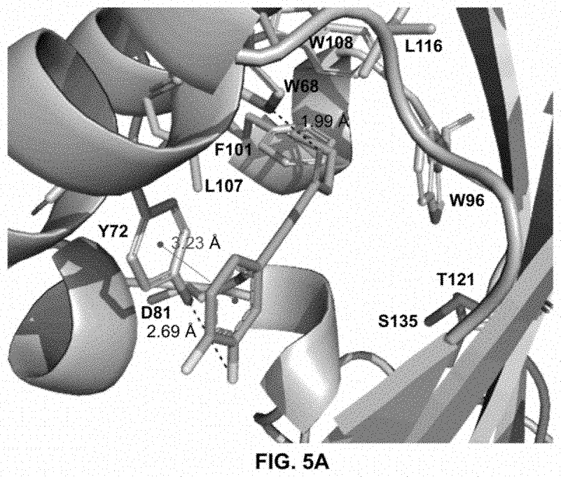

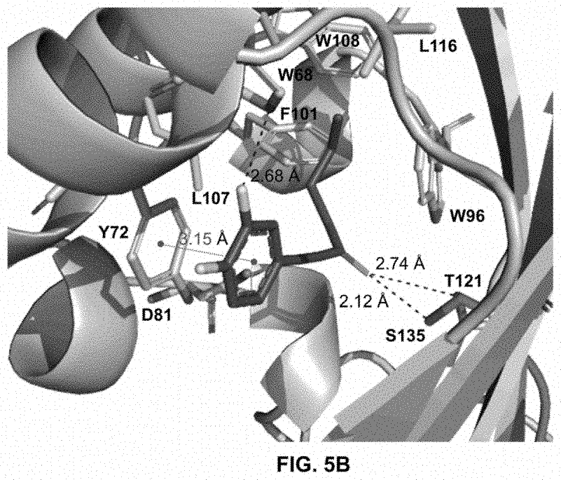

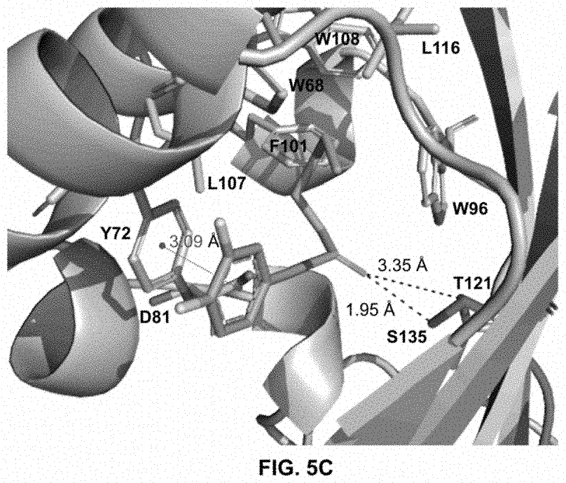

To explain how Compound 30 binds to RhlR, in silico molecular docking studies for Compound 30 and Compound 1d were performed using an RhlR homology model. As a result, it was confirmed that Compound 30 interacts with RhlR through a π-π stacking interaction with Tyr72, and has a hydrogen bond with Trp68, and Compound 1d has hydrogen bonds with Asp81, Thr121, and Ser135 (FIG. 4). Trp68 has been reported as a critical amino acid in BHL-independent and active rhlR variants (here, the rhlR variant refers to a variant in which the QS-related gene rhlR is knocked out), and in the present invention, and it was confirmed that as the length of an alkyl chain increases (Compounds 30, 31, and 32), the n-n stacking interaction with Tyr72 of a phenyl moiety is weaker (FIG. 5).

[In Vitro-Relative Biological Activity Result]

To prove the SAR results under the same experimental conditions and compare activities, among the above-described compounds, the representative compounds (Compound 11a, 11c, 11k, 11o, 13a, 30-32, and 33a to 33c) of the present invention were selected, and as relative biological activities, RhlR antagonistic activity, RhlR agonistic activity, static biofilm formation, bacterial growth, and rhamnolipid production are shown in Table 3 below. When a 4′-OH group of the phenyl ring was removed, the antagonistic activity of RhlR was significantly reduced (Compound 11c), and when a 3′-OMe of the phenyl ring is replaced with 3′-OH, RhlR antagonistic activity was increased (Compound 11k). As a result of comparing Compounds 11o and 13a, the presence or absence of a β-hydroxyl group in the middle is not essential for RhlR activity. The alkynyl ketone compounds having rotational rigidity (Compounds 30 to 32) were increased in RhlR activity. In addition, the (R)-alkynol (Compound 33b) has higher RhlR antagonistic activity than the (S)-alkynol (Compound 33c) and the racemate (Compound 33a). As shown in Table 3, the above-described synthetic compounds did not show RhlR agonistic activity even at 1000 μM. Meanwhile, the conventionally-reported RhlR antagonist (Compound 1d) had a relative RhlR agonistic activity of 37% at 1000 μM. Compared with a control, Compounds 11o, 13a, 30, 31, and 32 significantly inhibited biofilm formation at 100 μM. Particularly, Compound 30 relatively inhibited approximately 47% of the biofilm formation of P. aeruginosa, and is more potent than (S)-6-gingerol (Compound 1c) and the conventional antagonist (Compound 1d). In addition, as a result of measuring the bacterial growth inhibitory effect of the compounds of the present invention, Compounds 31 and 32 slightly inhibited cell growth at 100 PM, but Compound 30 had no effect on cell growth at the same concentration. Moreover, RhlR is known to directly regulate the expression of a rhamnolipid production enzyme, which is a representative virulence factor of P. aeruginosa, and as a result of measuring rhamnolipid production, Compound 30 (relative RhlR activity of 13%), which is the most potent RhlR antagonist, showed the lowest rhamnolipid production (42%) among the selected compounds. In addition, Compound 30 did not inhibit bacterial growth and more strongly inhibited biofilm formation and rhamnolipid production than other compounds which have strong RhlR activity but do not belong to the representative compounds (Compounds 7, 8, 11d, 11f, 21, 23a, 23b, and 23c) (Table 4).

TABLE 3

| | |
|---|---|
| 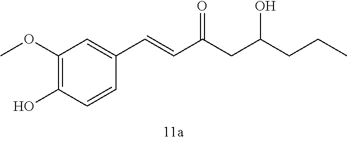 [SMILES: CCCC(O)CC(=O)/C=C/c1ccc(O)c(OC)c1] | 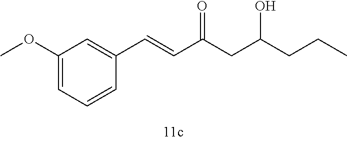 [SMILES: CCCC(O)CC(=O)/C=C/c1cccc(OC)c1] |
| 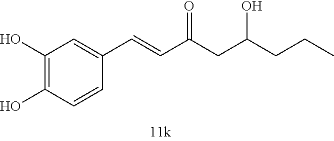 [SMILES: CCCC(O)CC(=O)/C=C/c1ccc(O)c(O)c1] | 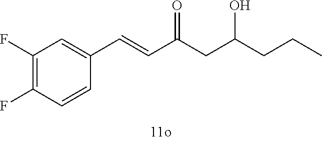 [SMILES: CCCC(O)CC(=O)/C=C/c1ccc(F)c(F)c1] |
| 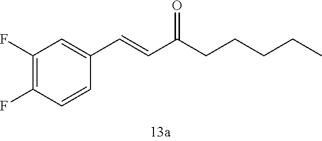 [SMILES: CCCCCC(=O)/C=C/c1ccc(F)c(F)c1] | 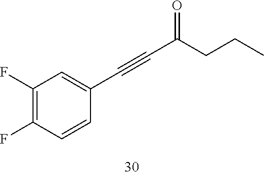 |
| 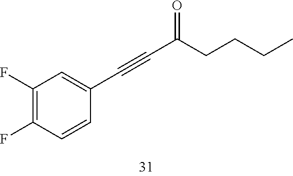 [SMILES: CCCCC(=O)C#Cc1ccc(F)c(F)c1] | 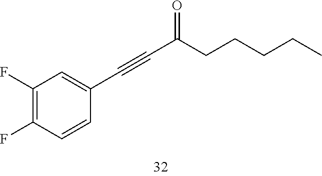 [SMILES: CCCCCC(=O)C#Cc1ccc(F)c(F)c1] |
| 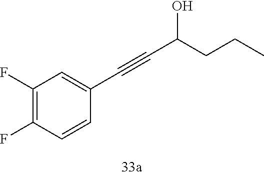 | 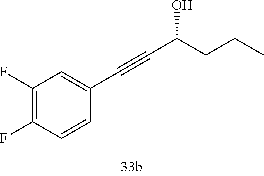 |

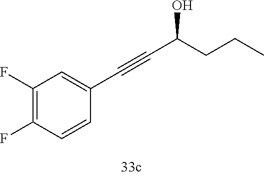

| | Relative RhIR Activity (%) | | | | | | Relative biological activity (%) | | | |
|---|---|---|---|---|---|---|---|---|---|---|
| Compound^{a} | Antagonism^{b} | | Agonism^{c} | | Biofilm formation^{d} | | Bacterial growth^{e} | | Rhamnolipid production^{f} | |
| | 10 | 100 | 100 | 1000 | 10 | 100 | 10 | 100 | 10 | 100 |
| 1c | 86* | 75** | 3** | 5** | 86 | 71** | 96* | 101 | 76* | 68* |
| 1d | 74* | 54** | 4** | 37** | 84 | 69** | 95* | 98 | 58** | 45** |
| 11a | 73** | 65** | 4** | 5** | 82 | 65** | 102 | 95 | 73* | 57* |
| 11c | 89 | 94 | 2** | 4** | 88 | 73** | 96 | 95* | 91 | 83 |
| 11k | 60** | 43** | 3** | 5** | 84* | 72** | 99 | 104* | 56* | 50* |
| 11o | 60** | 20** | 2** | 5** | 72* | 53** | 101 | 105* | 63* | 50** |
| 13a | 53** | 51** | 4** | 3** | 80* | 61** | 103 | 103 | 64** | 62* |
| 30 | 31** | 12** | 4** | 4** | 68* | 47** | 103 | 99 | 57** | 42* |
| 31 | 29** | 16** | 4** | 6** | 75* | 55** | 100 | 89** | 59** | 49** |
| 32 | 39** | 17** | 3** | 5** | 71* | 56** | 100 | 88** | 60** | 58** |
| 33a | 45** | 32** | 3** | 5** | 73** | 60** | 100 | 104* | 73* | 49** |
| 33b | 45** | 25** | 3** | 5** | 74* | 60** | 98 | 102 | 60* | 48** |
| 33c | 59** | 38** | 3** | 5** | 79* | 64** | 98 | 99 | 59* | 52** |

TABLE 4

| | | | Relative biological activity (%) | | |
|---|---|---|---|---|---|
| | Relative RhIR activity (%) | | Biofilm | Bacterial | Rhamnolipid |
| Compound [symbol] | Antagonism^{b} | Agonism^{c} | formation^{d} | growth [symbol] | production^{f} |
| 1c | 75** | 3** | 81* | 97 | 75* |
| 1d | 54** | 4** | 81* | 95 | 68* |
| 7 | 41** | 2** | 79** | 89 | 69* |
| 8 | 36** | 2** | 80** | 80** | 60* |
| 11a | 65** | 4** | 81* | 101 | 67* |
| 11c | 94 | 2** | 82* | 101 | 95 |
| 11d | 51** | 1** | 74* | 105 | 83 |
| 11f | 55** | 1** | 75* | 102 | 88 |
| 21 | 29** | 3** | 72* | 102 | 81* |
| 23a | 32** | 3** | 80* | 98 | 85 |
| 23b | 27** | 3** | 79* | 100 | 77* |
| 23c | 34** | 3** | 67* | 104 | 86 |
| 30 | 12** | 4** | 68* | 102 | 51* |

[symbol]

indicates data missing or illegible when filed

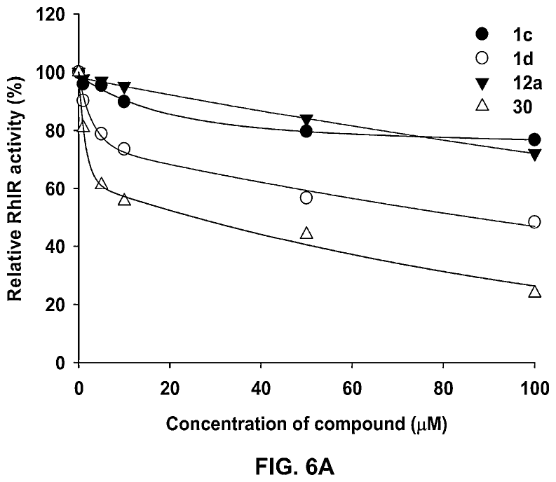

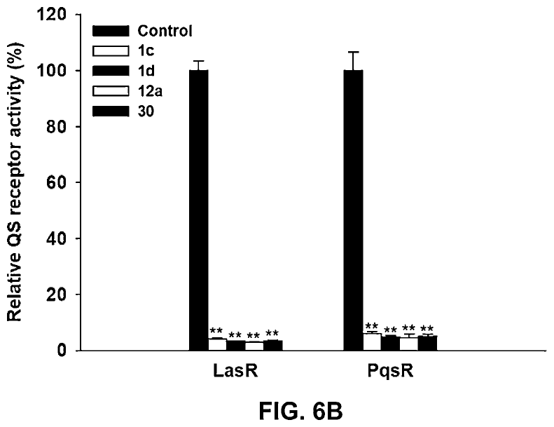

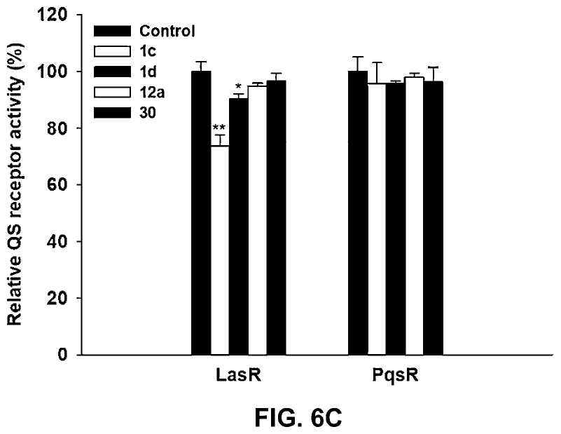

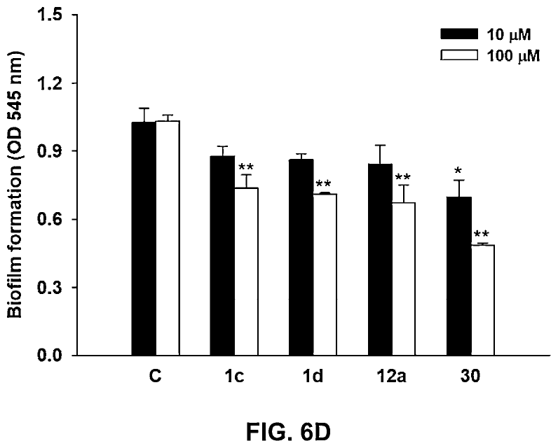

In the present invention, a comprehensive biological analysis of Compound 30, which is the most potent RhlR antagonist against P. aeruginosa, was performed. Here, Compounds 1c, 1d, and 4-gingerol (Compound 12a) were used as positive controls. First, the RhlR IC₅₀ values of the four compounds were measured at different concentrations (0 to 100 μM). As a result, the IC₅₀ values of Compounds 1c, 1d, 12a, and 30 were 945, 86, 218, and 26 μM, respectively (FIG. 6A), confirming that Compound 30 is the most potent RhlR antagonist. In addition, in the present invention, it was confirmed that the agonistic and antagonistic activities of the compounds against LasR and PqsR of the las and pqs systems were involved in the QS system of P. aeruginosa. The four compounds exhibited neither agonistic activity to LasR and PqsR nor antagonistic activity to PqsR at 0.1 and 10 μM (FIGS. 6B and 6C). However, the LasR antagonistic activity of (S)-6-gingerol (Compound 1c) was reported previously, and in the present invention, it was confirmed that 4-gingerol (Compound 12a) has no LasR antagonistic activity, which was consistent with the result of previous studies. Since Compound 30, which is the most potent RhlR antagonist, showed higher selectivity for RhlR than LasR and PqsR, it was identified as a pure RhlR antagonist.

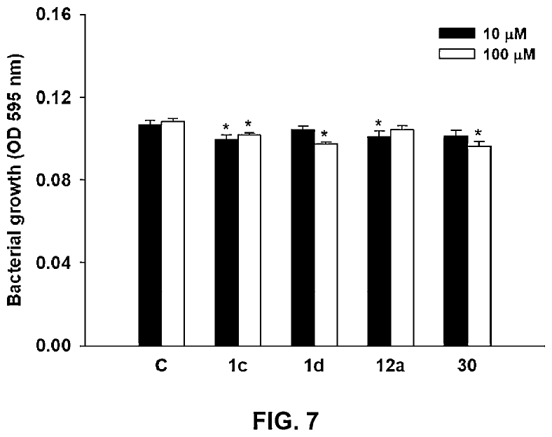

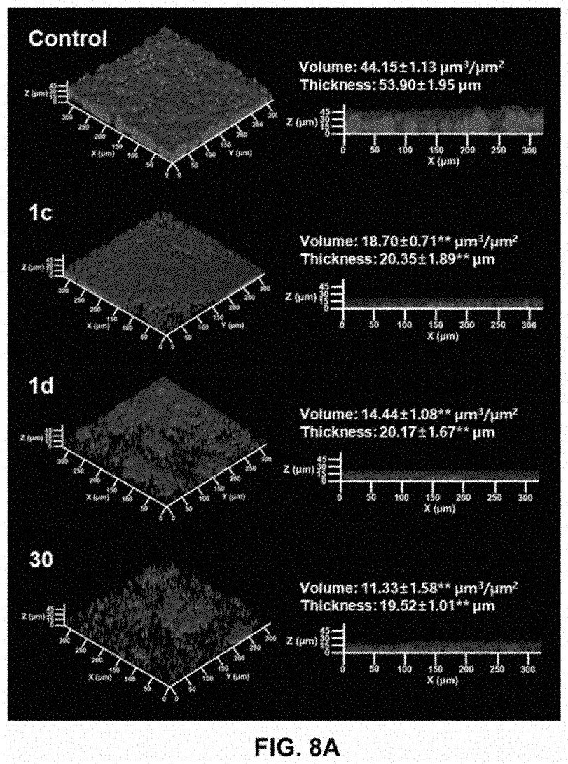

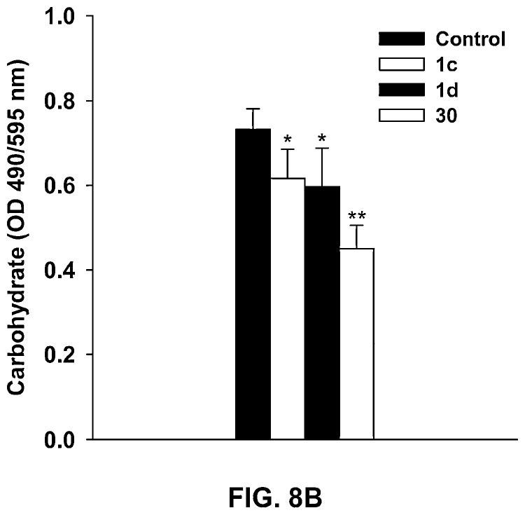

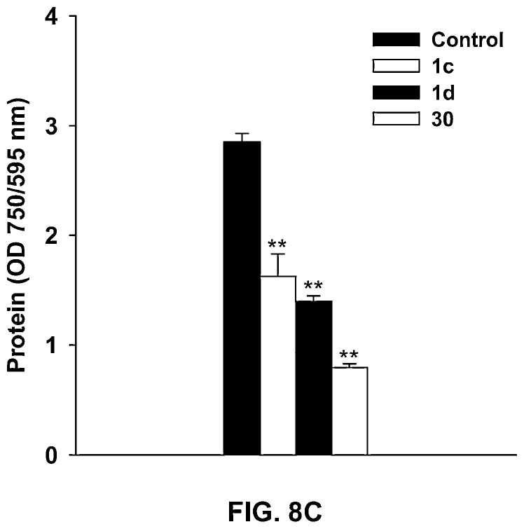

Meanwhile, the four compounds did not affect bacterial growth (FIG. 7), and strongly inhibited the biofilm formation of P. aeruginosa by 14 to 32% at 10 μM and by 29 to 53% at 100 μM (FIG. 6D). Dynamic biofilm inhibition was analyzed for 48 hours using a drip flow reactor. A control exhibited a volume of 44.15 μm³/μm² and a thickness of 53.90 μm in a typical mushroom shape (FIG. 8A). When other compounds (Compounds 1c, 1d, and 30) were treated, a thinner and more dispersed biofilm was formed, which had a smaller volume and thickness than the control. Particularly, Compound 30 inhibited biofilm formation by 74%, and reduced the amounts of carbohydrates and proteins by 39 and 72%, respectively (FIGS. 8B and 8C).

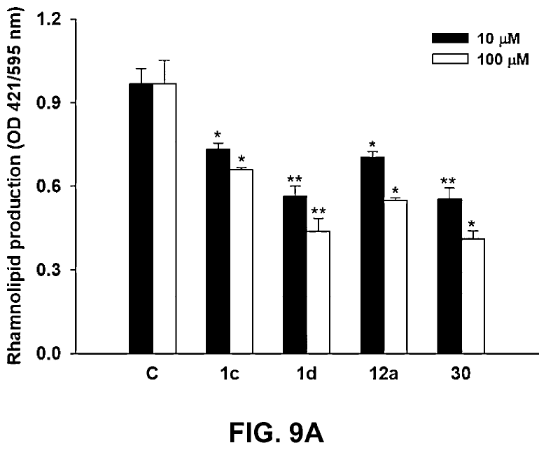

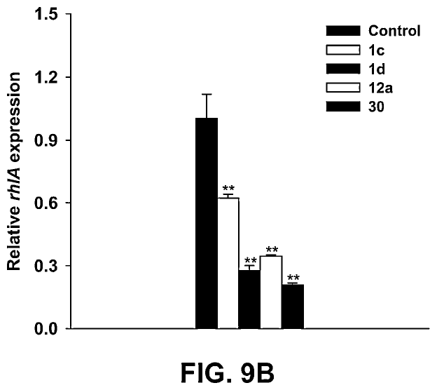

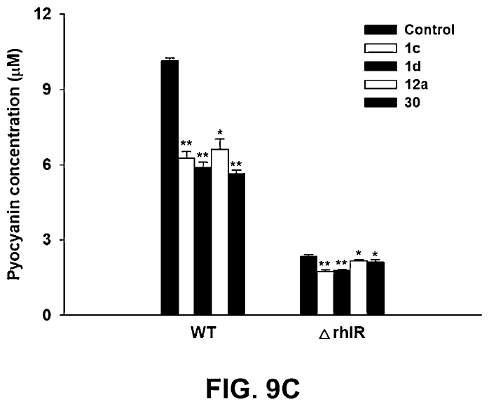

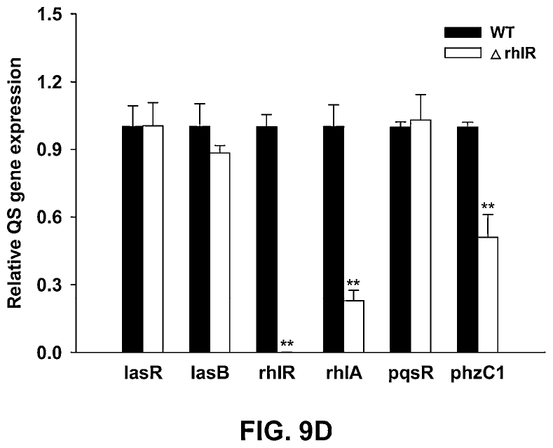

In the virulence factor production experiment, Compound 30 significantly reduced the rhamnolipid production of P. aeruginosa at 10 and 100 μM (FIG. 9A), and thus it was confirmed that the RhlR antagonists regulate rhamnolipid production, and plays a critical role in the biofilm growth stage. Generally, rhamnolipids increase cell hydrophobicity and bacterial twitching motility, thus affecting attachment and maturation in the biofilm formation stage. The P. aeruginosa rhlA variant downregulating rhamnolipid synthesis did not form a mushroom-shaped biofilm, indicating that the inhibition of rhamnolipid production induced by an RhlR antagonist can regulate the biofilm formation of P. aeruginosa (Here, the rhlA variant is a variant in which rhlA, which is a rhamnolipid synthesis gene, is knocked out).

In the present invention, it was confirmed that the inhibition of biofilm formation and virulence factor production induced by Compound 30 was closely associated with the rhl system by measuring the expression of a rhamnolipid synthesis gene (rhlA) in Compound 30-treated biofilm cells using RT-qPCR (FIG. 9B). The RhlR antagonists (Compounds 1d, 12a, and 30) significantly downregulate (65-79%) the rhlA expression of biofilm cells compared to Compound 1c (38%), showing that RhlR antagonists can regulate rhlA expression. That is, in the present invention, from the RT-qPCR result, it was confirmed that Compound 30 inhibits biofilm formation and virulence factor production by downregulating the rhlA expression of P. aeruginosa. Meanwhile, it was reported that the rhl system negatively regulates the pqs system to induce an increase in pyocyanin production, and considering the relationship between the rhl and pqs systems, a RhlR agonist was developed to be used as an anti-virulence strategy against pathogens, and there is prior research in which RhlR agonists inhibit pqs signaling to strongly inhibit pyocyanin production. Surprisingly, the rhlR variant of the present invention produced less pyocyanin than wild-type P. aeruginosa (FIG. 9C). In addition, Compound 30 reduced pyocyanin production by 44%, compared to the control. However, there was no difference in virulence factor production between Compound 30 and the control of the rhlR variant (FIG. 9C), which is because Compound 30 reduces pyocyanin production by interfering with RhlR. Such a difference is associated with the step of producing pyocyanin in P. aeruginosa. RhlR agonists are known to suppress the pqs system immediately before the production of basal pyocyanin. In prior research, the rhlR variant suppressed the rhl system-related genes, particularly, rhamnolipid production-related genes (rhlA and rhlB), but had little effect on the las- or pqs-related genes. A pyocyanin production-related gene (phzA-D) was also downregulated in the rhlR variant. In other prior research, in the rhlR variant, the downregulation of a pyocyanin biochemical gene (phzA1-G1) and reduced pyocyanin production have been reported. Likewise, according to the RT-qPCR experimental result of the present invention, the rhlR variant does not affect the expression of other QS-related genes (lasR, lasB, and pqsR), and reduces rhlA and phzC1 expression (p<0.005; FIG. 9D).

[In Vivo-Relative Biological Activity Result]

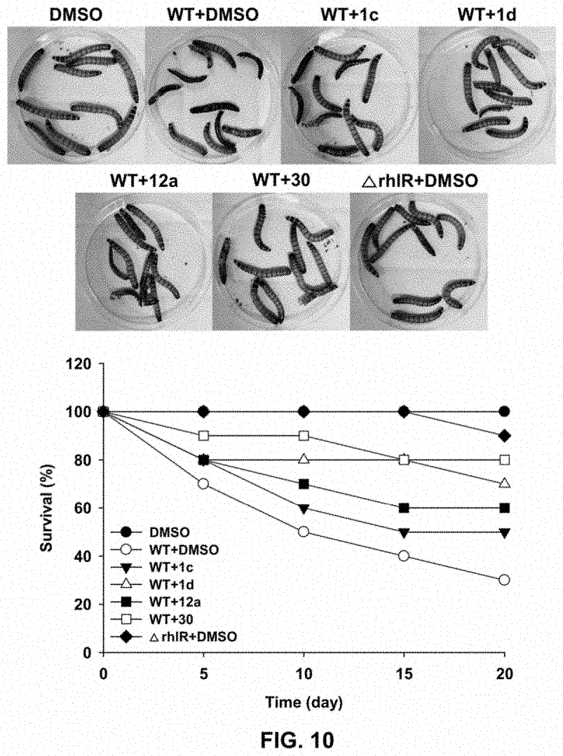

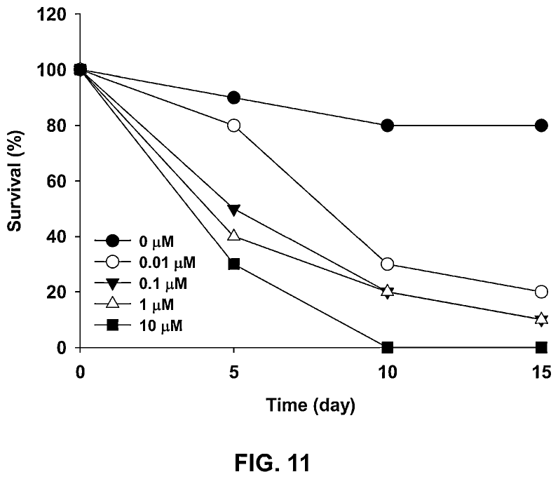

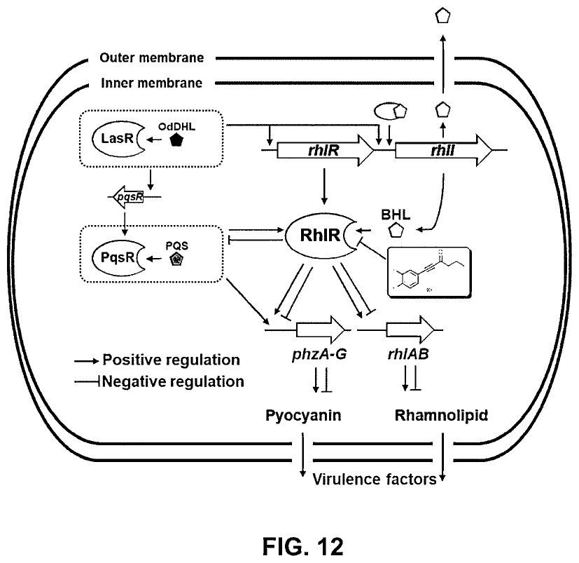

In the present invention, the effect of Compound 30 on the mortality of Tenebrio molitor larvae was analyzed. As a result of injecting P. aeruginosa into the larvae, the larvae started to die from the time of first incubation, and 70% of the larvae were killed within 20 days of incubation (FIG. 10). The survival rate of the larvae treated with Compound 30 was significantly improved and thus approximately 80% of the larvae survived to the end of the incubation. In addition, the survival rate of the larvae into which the rhlR variant of P. aeruginosa was injected was 90% after 20 days of incubation. In addition, when the larvae were treated with the rhlR variant with various concentrations (0.01 to 10 μM) of rhamnolipids, the mortality thereof was increased according to the concentration of rhamnolipids, confirming that RhlR plays a significant role in virulence factor production (FIG. 11). Therefore, Compound 30, which is the pure RhlR antagonist of the present invention, inhibited rhamnolipids and pyocyanin production by inactivation of the expression of virulence factor production genes (FIG. 12).

As above, as specific parts of the specification have been described in detail, although it is clear to those skilled in the art that these specific techniques are merely a preferred embodiment, the scope of the specification is not limited thereto. Thus, the substantial scope of the specification will be defined by the accompanying claims and their equivalents.

### INDUSTRIAL APPLICABILITY

In the present invention, a 4-gingerol derivative compound which can inhibit biofilm formation and virulence factor production, or a racemate, enantiomer or pharmaceutically acceptable salt thereof has a significantly improved binding affinity to RhlR and RhlR antagonistic activity thereby, and thus has an effect of effectively inhibiting biofilm formation and virulence factor production. Therefore, the compound or salt thereof can be effectively used in the field of treating various bacterial infections caused by a biofilm.

1. A gingerol derivative compound represented by Formula 1 below, a racemate, enantiomer or pharmaceutically acceptable salt thereof: 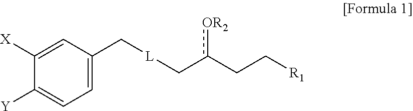
In Formula 1,
X and Y are the same or different from each other, and each independently any one selected from hydrogen, a halogen group, an alkyl group, a hydroxyl group, O—R′, and NR′R′ (R′ is a C1-C2 alkyl group), R₁ is a C1-C3 alkyl group,
the “.” represents a single bond or a double bond (however, when the “.” is a double bond, OR₂ is O, and when the “.” is a single bond, OR₂ is OH), and
L is any one selected from a single bond, a double bond and a triple bond.

2. The compound of claim 1, wherein the gingerol derivative compound represented by Formula 1 is any one selected from derivatives represented by the following Formulas 13a and 13b to Formulas 15a and 15b, Formula 16a, Formula 21, Formula 23, Formula 25, Formula 27, and Formula 30 to Formula 35, a racemate, enantiomer or a pharmaceutically available salt thereof:

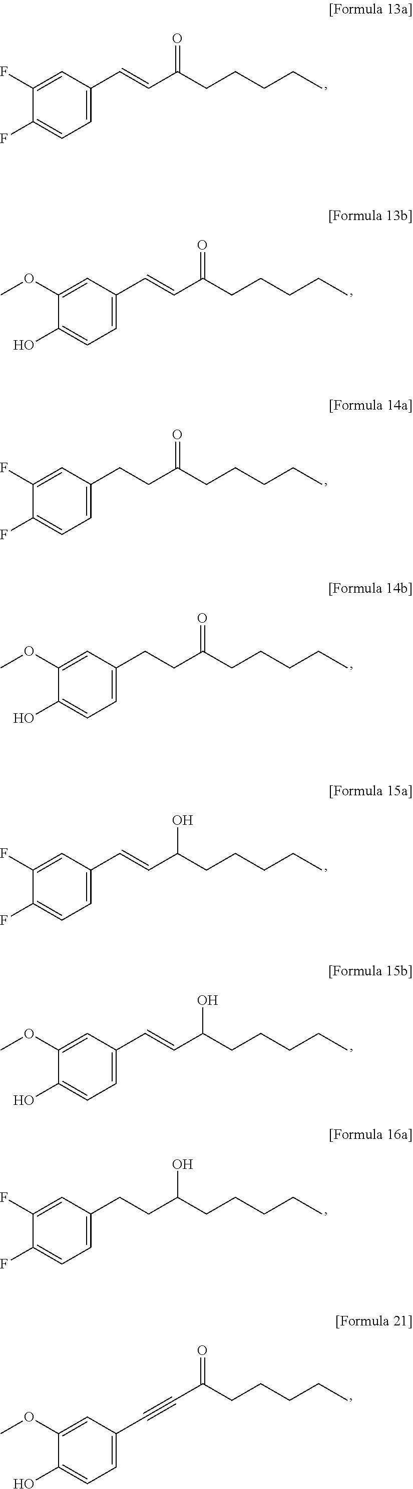

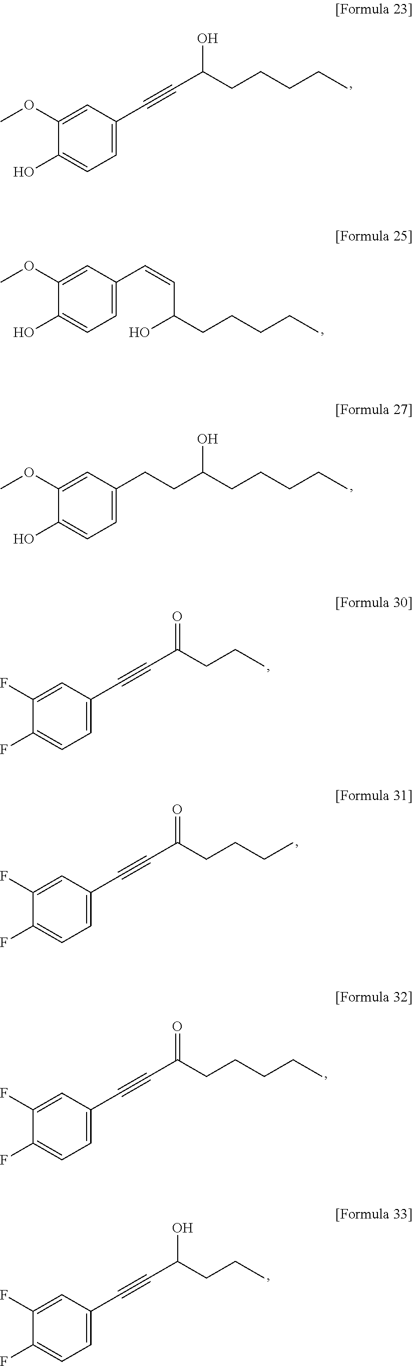

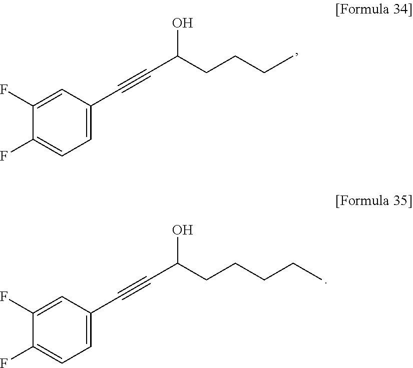 [SMILES: CCCCC(O)C#Cc1ccc(F)c(F)c1 ; CCCCCC(O)C#Cc1ccc(F)c(F)c1]

3. A gingerol derivative compound represented by Formula 2 below, a racemate, enantiomer or pharmaceutically acceptable salt thereof: 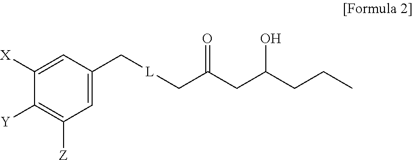
In Formula 2,
X, Y and Z are the same or different from each other, each independently any one selected from hydrogen, a halogen group, a hydroxyl group, an amino group, and O—R′ (R′ is a C1-C2 alkyl group), and
L is a single bond or a double bond.

4. The compound of claim 3, wherein the gingerol derivative compound represented by Formula 2 is any one selected from combinations of X, Y, and Z:
(1) X═OMe, Y═OH, Z═H
(2) X═H, Y═H, Z═H
(3) X═OMe, Y═H, Z═H
(4) X═H, Y═OH, Z═H
(5) X═OH, Y═H, Z═H
(6) X═H, Y═OMe, Z═H
(7) X═H, Y═F, Z═H
(8) X═H, Y═Cl, Z═H
(9) X═H, Y=Me, Z═H
(10) X═H, Y═NMe₂, Z═H
(11) X═OH, Y═OH, Z═H
(12) X═OMe, Y═OMe, Z═H
(13) X═F, Y═OH, Z═H
(14) X═OEt, Y═OH, Z═H
(15) X═F, Y═F, Z═H
(16) X=Me, Y=Me, Z═H
(17) X═OMe, Y═F, Z═H
(18) X═F, Y═H, Z═OMe

5. The compound of claim 1, which inhibits biofilm formation and virulence factor production.

6. The compound of claim 5, wherein the biofilm is formed by one or more species of bacteria selected from the group consisting of Pseudomonas aeruginosa, Salmonella spp, Shigella spp, Vibrio parahaemolyticus, Vibrio choreae, Escherichia coli O-157, Campylobacter jejuni, Clostridium difficile, Clostridium perfringens, Yersinia enterocolitica, Helicobacter pylori, Entemoeba histolytica, Bacillusu cereus, Clostridium botulinum, Haemophilus influenzae, Streptococcus pneumoniae, Chlamidia pneumoniae, Legionella pneumoniae, Branhamella catarrhalis, Mycobacterium tuberculosis, Mycoplasma pneumoniae, Group A Streptococcus (Streptococcus pyogenes), Corynebacterium diphtheriae, Bordetella pertussis, Chramidia psittaci, methicillin resistant Staphylococcus aureus (MRSA), Escherichia coli, Klebsiella pneumoniae, Enterobacter spp, Proteus spp, Acinetobacter spp, Enterococcus faecalis, Staphylococcus saprophyticus, and Group B Streptococcus (Streptococcus agalactiae).

7. A method of inhibiting biofilm formation, comprising the gingerol derivative compound of claim 1, or a racemate, enantiomer or a pharmaceutically acceptable salt thereof.

8. The method of claim 7, wherein the gingerol derivative compound has RhlR binding affinity.

9. A method of preventing or treating a bacterial infection, comprising:
administering the gingerol derivative compound of claim 1, a racemate, enantiomer or pharmaceutically acceptable salt thereof as an active ingredient.

10. The method of claim 9, wherein the bacterial infection is one or more types of infectious diseases selected from the group consisting of cystic fibrosis, pneumonia, tooth decay, periodontitis, otitis media, musculoskeletal infection, necrotizing fasciitis, biliary tract infection, osteomyelitis, bacterial prostatitis, native valve endocarditis, melioidosis, nosocomial infection, ICU pneumonia, urinary catheter cystitis, continuous ambulatory peritoneal dialysis (CAPD) peritonitis, and biliary stent blockage.

11. The method of claim 9, wherein the gingerol derivative compound downregulates rhlA expression.

12. A method of preventing or treating a bacterial infection, comprising:
administering the gingerol derivative compound of claim 3, a racemate, enantiomer or pharmaceutically acceptable salt thereof into a subject.

13. The method of claim 12, wherein the bacterial infection is one or more types of infectious diseases selected from the group consisting of cystic fibrosis, pneumonia, tooth decay, periodontitis, otitis media, musculoskeletal infection, necrotizing fasciitis, biliary tract infection, osteomyelitis, bacterial prostatitis, native valve endocarditis, melioidosis, nosocomial infection, ICU pneumonia, urinary catheter cystitis, continuous ambulatory peritoneal dialysis (CAPD) peritonitis, and biliary stent blockage.

14. The method of claim 12, wherein the gingerol derivative compound downregulates rhlA expression.

15. The compound of claim 3, which inhibits biofilm formation and virulence factor production.

16. The compound of claim 15, wherein the biofilm is formed by one or more species of bacteria selected from the group consisting of Pseudomonas aeruginosa, Salmonella spp, Shigella spp, Vibrio parahaemolyticus, Vibrio choreae, Escherichia coli O-157, Campylobacter jejuni, Clostridium dificile, Clostridium perfringens, Yersinia enterocolitica, Helicobacter pylori, Entemoeba histolytica, Bacillusu cereus, Clostridium botulinum, Haemophilus influenzae, Streptococcus pneumoniae, Chlamidia pneumoniae, Legionella pneumoniae, Branhamella catarrhalis, Mycobacterium tuberculosis, Mycoplasma pneumoniae, Group A Streptococcus (Streptococcus pyogenes), Corynebacterium diphtheriae, Bordetella pertussis, Chramidia psittaci, methicillin resistant Staphylococcus aureus (MRSA), Escherichia coli, Klebsiella pneumoniae, Enterobacter spp, Proteus spp, Acinetobacter spp, Enterococcus faecalis, Staphylococcus saprophyticus, and Group B Streptococcus (Streptococcus agalactiae).

17. A method of inhibiting biofilm formation, comprising the gingerol derivative compound of claim 3, or a racemate, enantiomer or a pharmaceutically acceptable salt thereof.

18. The method of claim 17, wherein the gingerol derivative compound has RhlR binding affinity.
